(19) United States
(12) Patent Application Publication
(10) Pub. No.: US 2026/0028629 A1
(43) Pub. Date: Jan. 29, 2026

(54) AGENTS FOR MODULATING EXPRESSION

(71) Applicant: Kicho Inc., New York, NY (US)

(72) Inventors: Oliver S.C. Quigley, New York, NY (US); Ethan Perlstein, Oakland, CA (US)

(21) Appl. No.: 18/872,481

(22) PCT Filed: Jun. 7, 2023
(86) PCT No.: PCT/US2023/024700

§ 371 Date: Dec. 6, 2024

Related U.S. Application Data
(60) Provisional application No. 63/349,659, filed on Jun. 7, 2022.

(51) Int. Cl. C12N 15/113; A61K 31/712; A61K 31/7125

(52) U.S. Cl. CPC C12N 15/1137; A61K 31/712; A61K 31/7125; C12N 2310/11; C12N 2310/14; C12N 2310/315; C12N 2310/321; C12Y 203/02

(57) ABSTRACT

Agents that target a nucleic acid (e.g., processed mRNA) can modulate expression of a protein that is encoded by the nucleic acid, e.g., via modulation of the level of the nucleic acid. In aspects, provided herein are compositions, methods, kits, and systems related to agents that modulate protein expression by targeting a nucleic acid molecule that encodes the protein.

### CROSS-REFERENCE

This application is a national phase entry of International Application No. PCT/US2023/024700, filed Jun. 7, 2023, which claims the benefit of U.S. Provisional Application No. 63/349,659, filed Jun. 7, 2022, each of which is incorporated herein by reference in its entirety.

### SEQUENCE LISTING

The instant application contains a Sequence Listing which has been submitted electronically in XML format and is hereby incorporated by reference in its entirety. Said XML copy, created on Jul. 17, 2023, is named 60868-701_601_SL.xml and is 1,724,339 bytes in size.

### SUMMARY

Disclosed herein, in some aspects, is a method of reducing expression of a UBE3A protein in a mammalian cell having duplication, overexpression, or a gain-of-function mutation of a UBE3A gene that encodes the UBE3A protein, the method comprises contacting an agent or a vector encoding the agent to the mammalian cell, wherein the agent reduces a level of a processed mRNA encoding the UBE3A protein in the mammalian cell.

In some cases, the agent comprises a polynucleotide sequence that is at least 80% complementary to at least 8 contiguous nucleic acids of the sequence set forth in any one of SEQ ID NO: 93-120.

In some cases, the agent comprises a polynucleotide sequence that is at least 80% complementary to at least 8 contiguous nucleic acids of each mRNA transcript listed in Table 2.

In some cases, the agent comprises an antisense oligomer.

In some cases, the agent comprises an antisense oligomer with at least 80% identity to the sequence set forth in any one of SEQ ID NO: 1-92.

Disclosed herein, in some aspects, is a method of modulating expression of a UBE3A gene encoding a UBE3A protein in a mammalian cell, the method comprises contacting an agent or a vector encoding the agent to the mammalian cell, wherein the agent comprises a polynucleotide sequence that comprises an antisense oligomer with at least 80% identity to the sequence set forth in any one of SEQ ID NO: 1-92.

In some cases, the antisense oligomer comprises a backbone modification, a modified sugar moiety or a combination thereof.

In some cases, the antisense oligomer comprises a phosphorothioate linkage or a phosphorodiamidate linkage.

In some cases, the antisense oligomer comprises a phosphorodiamidate morpholino, a locked nucleic acid, a peptide nucleic acid, a 2′-O-methyl moiety, a 2′-Fluoro moiety, a 2′-O-methoxyethyl moiety, or a 2′-NMA moiety.

In some cases, the antisense oligomer comprises at least one modified sugar moiety.

In some cases, the antisense oligomer comprises at least one, two, three, four, five, or six modified nucleosides at a 5′ end of the antisense oligomer.

In some cases, the antisense oligomer comprises one, two, three, four, five, or six modified nucleosides at a 5′ end of the antisense oligomer.

In some cases, the antisense oligomer comprises one, two, three, four, five, or six 2′-O-methoxyethyl modified nucleosides at a 5′ end of the antisense oligomer.

In some cases, the antisense oligomer comprises at least one, two, three, four, five, or six modified nucleosides at a 3′ end of the antisense oligomer.

In some cases, the antisense oligomer comprises one, two, three, four, five, or six modified nucleosides at a 3′ end of the antisense oligomer.

In some cases, the antisense oligomer comprises one, two, three, four, five, or six 2′-O-methoxyethyl modified nucleosides at a 3′ end of the antisense oligomer.

In some cases, the antisense oligomer comprises three, four, five, or six 2′-O-methoxyethyl modified nucleosides at a 5′ end of the antisense oligomer; three, four, five, or six 2′-O-methoxyethyl modified nucleosides at a 3′ end of the antisense oligomer; and a phosphorothioate linkage between any two neighboring nucleosides of the antisense oligomer.

In some cases, the antisense oligomer comprises: a 5′ region consisting of three, four, five, or six linked nucleosides; a central region consisting of eight, nine, ten, eleven, or twelve linked nucleosides; and a 3′ region consisting of three, four, five, or six linked nucleosides; wherein each of the three, four, five, or six linked nucleosides in the 5′ region and each of three, four, five, or six linked nucleosides in the 3′ region comprise a modified sugar moiety, and wherein each of the eight, nine, ten, eleven, or twelve linked nucleosides in the central region is a deoxyribonucleoside.

In some cases, the antisense oligomer consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 12 to 50 nucleobases, 12 to 40 nucleobases, 12 to 35 nucleobases, 12 to 30 nucleobases, 12 to 25 nucleobases, 12 to 20 nucleobases, 12 to 15 nucleobases, 15 to 50 nucleobases, 15 to 40 nucleobases, 15 to 35 nucleobases, 15 to 30 nucleobases, 15 to 25 nucleobases, 15 to 20 nucleobases, 15 to 19 nucleobases, 15 to 18 nucleobases, 15 to 16 nucleobases, 16 to 20 nucleobases, 16 to 19 nucleobases, 16 to 18 nucleobases, 17 to 20 nucleobases, 17 to 19 nucleobases, or 18 to 20 nucleobases.

In some cases, the antisense oligomer is a modified oligonucleotide comprising the sequence set forth in any one of SEQ ID NO: 154-189 or 192-247.

In some cases, the vector comprises a viral vector encoding the agent.

In some cases, the viral vector comprises an adenoviral vector, adeno-associated viral (AAV) vector, lentiviral vector, Herpes Simplex Virus (HSV) viral vector, or retroviral vector.

In some cases, the antisense oligomer comprises a sequence with at least 80% identity to the sequence set forth in any one of SEQ ID NO: 1-92.

In some cases, the antisense oligomer comprises a sequence with at least 90% identity to the sequence set forth in any one of SEQ ID NO: 1-92.

In some cases, the antisense oligomer comprises a sequence with 100% identity to the sequence set forth in any one of SEQ ID NO: 1-92.

In some cases, the antisense oligomer consists of a sequence with at least 80% identity to the sequence set forth in any one of SEQ ID NO: 1-92.

In some cases, the antisense oligomer consists of a sequence with at least 90% identity to the sequence set forth in any one of SEQ ID NO: 1-92.

In some cases, the antisense oligomer consists of a sequence with 100% identity to the sequence set forth in any one of SEQ ID NO: 1-92.

In some cases, the method reduces a level of processed mRNA encoding the UBE3A protein in the mammalian cell.

In some cases, the level of the processed mRNA encoding the UBE3A protein in the mammalian cell contacted with the agent or the vector is decreased by about 10% to about 99%, about 10% to about 95%, about 10% to about 90%, about 10% to about 80%, about 10% to about 70%, about 10% to about 60%, about 10% to about 50%, about 10% to about 40%, about 10% to about 30%, about 10% to about 20%, about 20% to about 90%, about 20% to about 80%, about 20% to about 70%, about 20% to about 60%, about 20% to about 50%, about 20% to about 40%, about 20% to about 30%, about 30% to about 80%, about 30% to about 70%, about 30% to about 60%, about 30% to about 50%, about 40% to about 70%, about 40% to about 60%, about 40% to about 50%, about 50% to about 99%, about 50% to about 95%, about 50% to about 90%, about 50% to about 80%, about 50% to about 70%, about 50% to about 60%, about 60% to about 99%, about 60% to about 95%, about 60% to about 90%, about 60% to about 80%, about 60% to about 70%, about 70% to about 99%, about 70% to about 95%, about 70% to about 90%, about 70% to about 80%, about 80% to about 99%, about 80% to about 95%, about 80% to about 90%, about 90% to about 99%, about 90% to about 95%, or about 95% to about 99%, as compared to an otherwise same cell not contacted with the agent or the vector.

In some cases, the level of the processed mRNA encoding the UBE3A protein in the mammalian cell contacted with the agent or the vector is decreased by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%, as compared to an otherwise same mammalian cell not contacted with the agent or the vector.

In some cases, the method reduces a level of the UBE3A protein in the mammalian cell.

In some cases, the level of the UBE3A protein in the mammalian cell contacted with the agent or the vector is decreased by about 10% to about 99%, about 10% to about 95%, about 10% to about 90%, about 10% to about 80%, about 10% to about 70%, about 10% to about 60%, about 10% to about 50%, about 10% to about 40%, about 10% to about 30%, about 10% to about 20%, about 20% to about 90%, about 20% to about 80%, about 20% to about 70%, about 20% to about 60%, about 20% to about 50%, about 20% to about 40%, about 20% to about 30%, about 30% to about 80%, about 30% to about 70%, about 30% to about 60%, about 30% to about 50%, about 40% to about 70%, about 40% to about 60%, about 40% to about 50%, about 50% to about 99%, about 50% to about 95%, about 50% to about 90%, about 50% to about 80%, about 50% to about 70%, about 50% to about 60%, about 60% to about 99%, about 60% to about 95%, about 60% to about 90%, about 60% to about 80%, about 60% to about 70%, about 70% to about 99%, about 70% to about 95%, about 70% to about 90%, about 70% to about 80%, about 80% to about 99%, about 80% to about 95%, about 80% to about 90%, about 90% to about 99%, about 90% to about 95%, or about 95% to about 99%, as compared to an otherwise same cell not contacted with the agent or the vector.

In some cases, the level of the UBE3A protein in the mammalian cell contacted with the agent or the vector is decreased by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%, as compared to an otherwise same cell not contacted with the agent or the vector.

In some cases, the method comprises contacting the agent or the vector to a population of mammalian cells.

In some cases, the agent reduces a level of the processed mRNA encoding the UBE3A protein in the population of mammalian cells by at most about 75%, at most about 70%, at most about 60%, at most about 50%, at most about 40%, at most about 30%, or at most about 20%, as compared to an otherwise same population of mammalian cells not contacted with the agent or the vector.

In some cases, the agent reduces a level of the UBE3A protein in the population of mammalian cells by at most about 75%, at most about 70%, at most about 60%, at most about 50%, at most about 40%, at most about 30%, or at most about 20%, as compared to an otherwise same population of mammalian cells not contacted with the agent or the vector.

In some cases, the mammalian cell is ex vivo.

In some cases, the mammalian cell is in vivo.

In some cases, genome of the mammalian cell has a duplication of a genomic region that encompasses the UBE3A gene encoding the UBE3A protein.

In some cases, the mammalian cell is a human cell, and wherein genome of the cell has a duplication of chromosome 15q11.2-q13.1.

In some cases, the mammalian cell is obtained from a human subject suffering from Dup15q syndrome, or a progeny of a sample cell obtained from the human subject.

Disclosed herein, in some aspects, is an antisense oligomer that comprises a sequence with at least 80% identity to the sequence set forth in any one of SEQ ID NO: 1-92.

In some cases, the antisense oligomer comprises a backbone modification, a modified sugar moiety or a combination thereof.

In some cases, the antisense oligomer comprises a phosphorothioate linkage or a phosphorodiamidate linkage.

In some cases, the antisense oligomer comprises a phosphorodiamidate morpholino, a locked nucleic acid, a peptide nucleic acid, a 2′-O-methyl moiety, a 2′-Fluoro moiety, a 2′-O-methoxyethyl moiety, or a 2′-NMA moiety.

In some cases, the antisense oligomer comprises at least one modified sugar moiety.

In some cases, the antisense oligomer comprises at least one, two, three, four, five, or six modified nucleosides at a 5′ end of the antisense oligomer.

In some cases, the antisense oligomer comprises one, two, three, four, five, or six modified nucleosides at a 5′ end of the antisense oligomer.

In some cases, the antisense oligomer comprises one, two, three, four, five, or six 2′-O-methoxyethyl modified nucleosides at a 5′ end of the antisense oligomer.

In some cases, the antisense oligomer comprises at least one, two, three, four, five, or six modified nucleosides at a 3′ end of the antisense oligomer.

In some cases, the antisense oligomer comprises one, two, three, four, five, or six modified nucleosides at a 3′ end of the antisense oligomer.

In some cases, the antisense oligomer comprises one, two, three, four, five, or six 2′-O-methoxyethyl modified nucleosides at a 3′ end of the antisense oligomer.

In some cases, the antisense oligomer comprises three, four, five, or six 2′-O-methoxyethyl modified nucleosides at a 5′ end of the antisense oligomer; three, four, five, or six 2′-O-methoxyethyl modified nucleosides at a 3′ end of the antisense oligomer; and a phosphorothioate linkage between any two neighboring nucleosides of the antisense oligomer.

In some cases, the antisense oligomer comprises: a 5′ region consisting of three, four, five, or six linked nucleosides; a central region consisting of eight, nine, ten, eleven, or twelve linked nucleosides; and a 3′ region consisting of three, four, five, or six linked nucleosides; wherein each of the three, four, five, or six linked nucleosides in the 5′ region and each of three, four, five, or six linked nucleosides in the 3′ region comprise a modified sugar moiety, and wherein each of the eight, nine, ten, eleven, or twelve linked nucleosides in the central region is a deoxyribonucleoside.

In some cases, the antisense oligomer consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 12 to 50 nucleobases, 12 to 40 nucleobases, 12 to 35 nucleobases, 12 to 30 nucleobases, 12 to 25 nucleobases, 12 to 20 nucleobases, 12 to 15 nucleobases, 15 to 50 nucleobases, 15 to 40 nucleobases, 15 to 35 nucleobases, 15 to 30 nucleobases, 15 to 25 nucleobases, 15 to 20 nucleobases, 15 to 19 nucleobases, 15 to 18 nucleobases, 15 to 16 nucleobases, 16 to 20 nucleobases, 16 to 19 nucleobases, 16 to 18 nucleobases, 17 to 20 nucleobases, 17 to 19 nucleobases, or 18 to 20 nucleobases.

In some cases, the antisense oligomer comprises a sequence with at least 80% identity to the sequence set forth in any one of SEQ ID NO: 1-92.

In some cases, the antisense oligomer comprises a sequence with at least 90% identity to the sequence set forth in any one of SEQ ID NO: 1-92.

In some cases, the antisense oligomer comprises a sequence with 100% identity to the sequence set forth in any one of SEQ ID NO: 1-92.

In some cases, the antisense oligomer consists of a sequence with at least 80% identity to the sequence set forth in any one of SEQ ID NO: 1-92.

In some cases, the antisense oligomer consists of a sequence with at least 90% identity to the sequence set forth in any one of SEQ ID NO: 1-92.

In some cases, the antisense oligomer consists of a sequence with 100% identity to the sequence set forth in any one of SEQ ID NO: 1-92.

In some cases, the antisense oligomer is a modified oligonucleotide comprising the sequence set forth in any one of SEQ ID NO: 154-189 or 192-247.

In some cases, the antisense oligomer is a modified oligonucleotide consisting of the sequence set forth in any one of SEQ ID NO: 154-189 or 192-247.

In some cases, the antisense oligomer is configured to reduce a level of a processed mRNA transcript encoding a UBE3A protein in a population of mammalian cells upon contact with the population.

In some cases, the antisense oligomer is configured to reduce the level of the processed mRNA encoding the UBE3A protein in the population by about 10% to about 99%, about 10% to about 95%, about 10% to about 90%, about 10% to about 80%, about 10% to about 70%, about 10% to about 60%, about 10% to about 50%, about 10% to about 40%, about 10% to about 30%, about 10% to about 20%, about 20% to about 90%, about 20% to about 80%, about 20% to about 70%, about 20% to about 60%, about 20% to about 50%, about 20% to about 40%, about 20% to about 30%, about 30% to about 80%, about 30% to about 70%, about 30% to about 60%, about 30% to about 50%, about 40% to about 70%, about 40% to about 60%, about 40% to about 50%, about 50% to about 99%, about 50% to about 95%, about 50% to about 90%, about 50% to about 80%, about 50% to about 70%, about 50% to about 60%, about 60% to about 99%, about 60% to about 95%, about 60% to about 90%, about 60% to about 80%, about 60% to about 70%, about 70% to about 99%, about 70% to about 95%, about 70% to about 90%, about 70% to about 80%, about 80% to about 99%, about 80% to about 95%, about 80% to about 90%, about 90% to about 99%, about 90% to about 95%, or about 95% to about 99%, as compared to an otherwise same population of mammalian cells not contacted with the antisense oligomer.

In some cases, the antisense oligomer is configured to reduce the level of the processed mRNA encoding the UBE3A protein in the population by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%, as compared to an otherwise same cell not contacted with the antisense oligomer.

In some cases, the antisense oligomer is configured to reduce the level of the processed mRNA encoding the UBE3A protein in the population by at most about 75%, at most about 70%, at most about 60%, at most about 50%, at most about 40%, at most about 30%, or at most about 20%, as compared to an otherwise same population of mammalian cells not contacted with the antisense oligomer.

In some cases, the antisense oligomer is configured to reduce a level of the UBE3A protein in the population of mammalian cells.

In some cases, the antisense oligomer is configured to reduce the level of the UBE3A protein in the population by about 10% to about 99%, about 10% to about 95%, about 10% to about 90%, about 10% to about 80%, about 10% to about 70%, about 10% to about 60%, about 10% to about 50%, about 10% to about 40%, about 10% to about 30%, about 10% to about 20%, about 20% to about 90%, about 20% to about 80%, about 20% to about 70%, about 20% to about 60%, about 20% to about 50%, about 20% to about 40%, about 20% to about 30%, about 30% to about 80%, about 30% to about 70%, about 30% to about 60%, about 30% to about 50%, about 40% to about 70%, about 40% to about 60%, about 40% to about 50%, about 50% to about 99%, about 50% to about 95%, about 50% to about 90%, about 50% to about 80%, about 50% to about 70%, about 50% to about 60%, about 60% to about 99%, about 60% to about 95%, about 60% to about 90%, about 60% to about 80%, about 60% to about 70%, about 70% to about 99%, about 70% to about 95%, about 70% to about 90%, about 70% to about 80%, about 80% to about 99%, about 80% to about 95%, about 80% to about 90%, about 90% to about 99%, about 90% to about 95%, or about 95% to about 99%, as compared to an otherwise same population of mammalian cells not contacted with the antisense oligomer.

In some cases, the antisense oligomer is configured to reduce the level of the UBE3A protein in the population by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%, as compared to an otherwise same population of mammalian cells not contacted with the antisense oligomer.

In some cases, the antisense oligomer is configured to reduce the level of the UBE3A protein in the population by at most about 75%, at most about 70%, at most about 60%, at most about 50%, at most about 40%, at most about 30%, or at most about 20%, as compared to an otherwise same population of mammalian cells not contacted with the antisense oligomer.

In some cases, the mammalian cell is ex vivo.

In some cases, the mammalian cell is in vivo.

In some cases, genome of the mammalian cell has a duplication of a genomic region that encompasses the UBE3A gene encoding the UBE3A protein.

In some cases, the mammalian cell is a human cell.

In some cases, genome of the mammalian cell has a duplication of chromosome 15q11.2-q13.1.

In some cases, the mammalian cell is obtained from a human subject suffering from Dupl5q syndrome, or a progeny of a sample cell obtained from the human subject.

Disclosed herein, in some aspects, is a pharmaceutical composition, comprising: (a) a pharmaceutically acceptable excipient or carrier; and (b) the antisense oligomer disclosed herein.

Disclosed herein, in some aspects, is a pharmaceutical composition, comprising: (a) a pharmaceutically acceptable excipient or carrier; and (b) an agent or a vector encoding the agent, wherein the agent is configured to reduce a level of a processed mRNA transcript encoding a UBE3A protein in a mammalian cell upon contact with the mammalian cell.

In some cases, the agent comprises a polynucleotide sequence that is at least 80% complementary to at least 8 contiguous nucleic acids of the sequence set forth in any one of SEQ ID NO: 93-120.

In some cases, the agent comprises a polynucleotide sequence that is at least 80% complementary to at least 8 contiguous nucleic acids of each mRNA transcript listed in Table 2.

In some cases, the agent comprises an antisense oligomer.

In some cases, the antisense oligomer has at least 80% identity to the sequence set forth in any one of SEQ ID NO: 1-92.

In some cases, the pharmaceutical composition comprises the vector, and wherein the vector comprises a viral vector encoding the agent.

In some cases, the viral vector comprises an adenoviral vector, adeno-associated viral (AAV) vector, lentiviral vector, Herpes Simplex Virus (HSV) viral vector, or retroviral vector.

In some cases, the antisense oligomer comprises a sequence with at least 80% identity to the sequence set forth in any one of SEQ ID NO: 1-92.

In some cases, the antisense oligomer comprises a sequence with at least 90% identity to the sequence set forth in any one of SEQ ID NO: 1-92.

In some cases, the antisense oligomer comprises a sequence with 100% identity to the sequence set forth in any one of SEQ ID NO: 1-92.

In some cases, the antisense oligomer consists of a sequence with at least 80% identity to the sequence set forth in any one of SEQ ID NO: 1-92.

In some cases, the antisense oligomer consists of a sequence with at least 90% identity to the sequence set forth in any one of SEQ ID NO: 1-92.

In some cases, the antisense oligomer consists of a sequence with 100% identity to the sequence set forth in any one of SEQ ID NO: 1-92.

In some cases, the antisense oligomer is a modified oligonucleotide comprising the sequence set forth in any one of SEQ ID NO: 154-189 or 192-247.

In some cases, the antisense oligomer is a modified oligonucleotide consisting of the sequence set forth in any one of SEQ ID NO: 154-189 or 192-247.

In some cases, the agent is configured to reduce the level of the processed mRNA encoding the UBE3A protein in the population of the mammalian cells upon contact with the population.

In some cases, the antisense oligomer is configured to reduce the level of the processed mRNA encoding the UBE3A protein in the population by about 10% to about 99%, about 10% to about 95%, about 10% to about 90%, about 10% to about 80%, about 10% to about 70%, about 10% to about 60%, about 10% to about 50%, about 10% to about 40%, about 10% to about 30%, about 10% to about 20%, about 20% to about 90%, about 20% to about 80%, about 20% to about 70%, about 20% to about 60%, about 20% to about 50%, about 20% to about 40%, about 20% to about 30%, about 30% to about 80%, about 30% to about 70%, about 30% to about 60%, about 30% to about 50%, about 40% to about 70%, about 40% to about 60%, about 40% to about 50%, about 50% to about 99%, about 50% to about 95%, about 50% to about 90%, about 50% to about 80%, about 50% to about 70%, about 50% to about 60%, about 60% to about 99%, about 60% to about 95%, about 60% to about 90%, about 60% to about 80%, about 60% to about 70%, about 70% to about 99%, about 70% to about 95%, about 70% to about 90%, about 70% to about 80%, about 80% to about 99%, about 80% to about 95%, about 80% to about 90%, about 90% to about 99%, about 90% to about 95%, or about 95% to about 99%, as compared to an otherwise same population of mammalian cells not contacted with the agent or the vector.

In some cases, the agent is configured to reduce the level of the processed mRNA encoding the UBE3A protein in the population by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%, as compared to an otherwise same population of mammalian cells not contacted with the agent or the vector.

In some cases, the agent is configured to reduce the level of the processed mRNA encoding the UBE3A protein in the population by at most about 75%, at most about 70%, at most about 60%, at most about 50%, at most about 40%, at most about 30%, or at most about 20%, as compared to an otherwise same population of mammalian cells not contacted with the antisense oligomer.

In some cases, the agent is configured to reduce a level of the UBE3A protein in the population.

In some cases, the agent is configured to reduce the level of the UBE3A protein in the population by about 10% to about 99%, about 10% to about 95%, about 10% to about 90%, about 10% to about 80%, about 10% to about 70%, about 10% to about 60%, about 10% to about 50%, about 10% to about 40%, about 10% to about 30%, about 10% to about 20%, about 20% to about 90%, about 20% to about 80%, about 20% to about 70%, about 20% to about 60%, about 20% to about 50%, about 20% to about 40%, about 20% to about 30%, about 30% to about 80%, about 30% to about 70%, about 30% to about 60%, about 30% to about 50%, about 40% to about 70%, about 40% to about 60%, about 40% to about 50%, about 50% to about 99%, about 50% to about 95%, about 50% to about 90%, about 50% to about 80%, about 50% to about 70%, about 50% to about 60%, about 60% to about 99%, about 60% to about 95%, about 60% to about 90%, about 60% to about 80%, about 60% to about 70%, about 70% to about 99%, about 70% to about 95%, about 70% to about 90%, about 70% to about 80%, about 80% to about 99%, about 80% to about 95%, about 80% to about 90%, about 90% to about 99%, about 90% to about 95%, or about 95% to about 99%, as compared to an otherwise same population of mammalian cells not contacted with the agent or the vector.

In some cases, the agent is configured to reduce the level of the UBE3A protein in the population by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%, as compared to an otherwise same population of mammalian cells not contacted with the agent or the vector.

In some cases, the agent is configured to reduce the level of the UBE3A protein in the population by at most about 75%, at most about 70%, at most about 60%, at most about 50%, at most about 40%, at most about 30%, or at most about 20%, as compared to an otherwise same population of mammalian cells not contacted with the antisense oligomer.

In some cases, the mammalian cell is ex vivo. In some cases, the mammalian cell is in vivo. In some cases, genome of the mammalian cell has a duplication of a genomic region that encompasses the UBE3A gene encoding the UBE3A protein.

In some cases, the mammalian cell is a human cell. In some cases, genome of the mammalian cell has a duplication of chromosome 15q11.2-q13.1.

In some cases, the mammalian cell is obtained from a human subject suffering from Dupl5q syndrome, or a progeny of a sample cell obtained from the human subject.

In some cases, the pharmaceutical composition is formulated for intracerebroventricular injection, intraperitoneal injection, intramuscular injection, intrathecal injection, intra cisterna magna injection, subcutaneous injection, oral administration, synovial injection, intravitreal administration, subretinal injection, topical application, implantation, or intravenous injection.

In some cases, the pharmaceutical composition is formulated for intrathecal injection.

In some cases, the pharmaceutically acceptable excipient or carrier comprises artificial cerebrospinal fluid.

In some cases, the pharmaceutical composition further comprises a second therapeutic agent.

In some cases, the second therapeutic agent comprises a small molecule, an antisense oligomer, or a gene editing molecule.

Disclosed herein, in some aspects, is a method of treating or reducing the likelihood of developing a disease or condition in a subject in need thereof by reducing expression of a UBE3A protein in cells of the subject, comprising contacting to the cells of the subject the pharmaceutical composition disclosed herein.

In some cases, the disease or condition is associated with overexpression or gain-of-function mutation in a UBE3A gene encoding the UBE3A protein.

In some cases, genomes of the cells of the subject have at least one excessive copy of a UBE3A gene encoding the UBE3A protein.

In some cases, genomes of the cells of the subject have a duplication of a genomic region encompassing a UBE3A gene encoding the UBE3A protein.

In some cases, genomes of the cells of the subject have a duplication of chromosome 15q11.2-q13.1.

In some cases, the disease or condition comprises Dup15q syndrome, autism spectrum disorder, epilepsy, or intellectual disability.

In some cases, the subject is a human. In some cases, the subject is a fetus, an embryo, or a child. In some cases, the cells are ex vivo.

In some cases, the method comprises administering the pharmaceutical composition to the subject by intracerebroventricular injection, intraperitoneal injection, intramuscular injection, intrathecal injection, intra cisterna magna injection, subcutaneous injection, oral administration, synovial injection, intravitreal administration, subretinal injection, topical application, implantation, or intravenous injection.

In some cases, the method comprises administering the pharmaceutical composition to the subject by intrathecal injection.

In some cases, the method treats the disease or condition.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-ID show concentration response curves (CRCs) for each of 22 exemplary ASOs according to some embodiments of the present disclosure, as measured by percentage knockdown of human UBE3A gene in the cells treated with respective ASOs.

FIGS. 2A-2B are histograms illustrating the effect of various controls on UBE3A mRNA knockdown.

FIGS. 3A-3B are histograms showing the effect of exemplary ASOs at two different concentrations (6.3 μM and 20 μM, respectively) on the knockdown of UBE3A mRNA level on treatment Day 7, treatment Day 10, and treatment Day 14.

FIGS. 3C-3D are histograms illustrating the effect of test ASOs at two different concentrations (6.3 μM and 20 μM, respectively) on the reduction of UBE3A protein expression on treatment Day 7, treatment Day 10, and treatment Day 14.

FIGS. 4A-4D are histograms illustrating the fold changes in mRNA and protein expression in F-Dup and Corrected neuron cells. FIG. 4A illustrates the relative UBE3A mRNA expression on differentiation day 11 of F-Dup and Corrected neuronal cells (normalized to Corrected neuronal cells). FIG. 4B illustrates the relative UBE3A protein expression on differentiation Day 11 of F-Dup and Corrected neuronal cells (normalized to Corrected neuronal cells). FIG. 4C illustrates the relative UBE3A mRNA expression on differentiation day 22 of the F-Dup and Corrected neuronal cells (normalized to Corrected neuronal cells). FIG. 4D illustrates the relative UBE3A protein expression on differentiation day 22 of F-Dup and Corrected neuronal cells (normalized to Corrected neuronal cells).

FIGS. 5A-5B illustrate the UBE3A protein expression levels on ASO treatment days 7 and 10 at 6.3 μM (FIG. 5A) and 20 μM (FIG. 5B) of the exemplary ASOs on F-Dup neurons on treatment Day 7, treatment Day 10, and treatment Day 14.

FIG. 6 is a graphical representation of the timeline for time neuronal cells spent in culture, duration of ASO treatment, and duration of wash-out culture media.

### DETAILED DESCRIPTION

The section headings used herein are for organizational purposes only and are not to be construed as limiting the subject matter described.

### Certain Terminology

Unless specific definitions are provided, the nomenclature utilized in connection with, and the procedures and techniques of, analytical chemistry, synthetic organic chemistry, and medicinal and pharmaceutical chemistry described herein are those well-known and commonly used in the art. Standard techniques may be used for chemical synthesis, and chemical analysis.

Unless otherwise indicated, the following terms have the following meanings.

“Administering” can mean providing a pharmaceutical agent to an animal, and includes, but is not limited to administering by a medical professional and self-administering. “Amelioration” refers to a lessening, slowing, stopping, or reversing of at least one indicator of the severity of a syndrome or condition. The severity of indicators may be determined by subjective or objective measures, which are known to those skilled in the art.

“Animal” can refer to a human or non-human animal, including, but not limited to, mice, rats, rabbits, dogs, cats, pigs, and non-human primates, including, but not limited to, monkeys and chimpanzees.

“Antisense oligomer” can mean an oligomeric compound that is capable of undergoing hybridization to a target nucleic acid through hydrogen bonding. Examples of antisense oligomers include single-stranded and double-stranded compounds, such as, antisense oligonucleotides, siRNAs, shRNAs, and ssRNAs.

“Antisense inhibition” or “inhibition” can mean reduction of target nucleic acid levels in the presence of an antisense oligomer complementary to a target nucleic acid compared to target nucleic acid levels or in the absence of the antisense oligomer.

“Antisense mechanisms” can refer to all those mechanisms involving hybridization of a compound with a target nucleic acid, wherein the outcome or effect of the hybridization is either target degradation or target occupancy with concomitant stalling of the cellular machinery involving, for example, transcription or splicing. An antisense oligomer provided herein can be “antisense” to a target nucleic acid, meaning that the antisense oligomer is capable of undergoing hybridization to a target nucleic acid through hydrogen bonding.

“Antisense oligonucleotide” can mean a single-stranded oligonucleotide having a nucleobase sequence that permits hybridization to a corresponding segment of a target nucleic acid.

“Base complementarity” can refer to the capacity for the precise base pairing of nucleobases of an antisense oligonucleotide with corresponding nucleobases in a target nucleic acid (i.e., hybridization), and is mediated by Watson-Crick, Hoogsteen or reversed Hoogsteen hydrogen binding between corresponding nucleobases.

“Bicyclic sugar” can mean a furanose ring modified by the bridging of two atoms. A bicyclic sugar is a modified sugar.

“Bicyclic nucleoside” (also “BNA”) can mean a nucleoside having a sugar moiety comprising a bridge connecting two carbon atoms of the sugar ring, thereby forming a bicyclic ring system. In certain embodiments, the bridge connects the 4′-carbon and the 2′-carbon of the sugar ring.

“Cap structure” or “terminal cap moiety” can mean chemical modifications, which have been incorporated at either terminus of an antisense oligomer. “cEt” or “constrained ethyl” can mean a bicyclic nucleoside having a sugar moiety comprising a bridge connecting the 4′-carbon and the 2′-carbon, wherein the bridge has the formula: 4′-CH(CH₃)—O-2′

“Constrained ethyl nucleoside” (also cEt nucleoside) can mean a nucleoside comprising a bicyclic sugar moiety comprising a 4′-CH(CH₃)—O-2′ bridge.

“Chimeric antisense oligomer” can mean an antisense oligomer that has at least two chemically distinct regions, each position having a plurality of subunits.

“Complementarity” can mean the capacity for pairing between nucleobases of a first nucleic acid and a second nucleic acid.

“Contiguous nucleobases” can mean nucleobases immediately adjacent to each other.

“Diluent” can mean an ingredient in a composition that lacks pharmacological activity, but is pharmaceutically necessary or desirable. For example, in drugs that are injected, the diluent may be a liquid, e.g., saline solution.

“Effective amount” in the context of modulating an activity or of treating or preventing a condition can mean the administration of that amount of pharmaceutical agent to an individual in need of such modulation, treatment, or prophylaxis, either in a single dose or as part of a series, that is effective for modulation of that effect, or for treatment or prophylaxis or improvement of that condition. The effective amount may vary among individuals depending on the health and physical condition of the individual to be treated, the taxonomic group of the individuals to be treated, the formulation of the composition, assessment of the individual's medical condition, and other relevant factors.

“Efficacy” or “potency,” which are used herein interchangeably, can mean the ability to produce a desired effect.

“Expression” can include all the processes by which a gene's coded information is converted into structures present and operating in a cell. Such structures include, but are not limited to the products of transcription and translation.

“Gapmer” can mean a chimeric antisense oligomer in which an internal region having a plurality of nucleosides that support RNase H cleavage is positioned between external regions having one or more nucleosides, wherein the nucleosides comprising the internal region are chemically distinct from the nucleoside or nucleosides comprising the external regions. The internal region can be referred to as a “gap” and the external regions can be referred to as the “wings.”

“Hybridization” can mean the annealing of complementary nucleic acid molecules. In certain embodiments, complementary nucleic acid molecules include, but are not limited to, an antisense oligomer and a target nucleic acid. In certain embodiments, complementary nucleic acid molecules include, but are not limited to, an antisense oligonucleotide and a nucleic acid target.

“Individual” can mean a human or non-human animal selected for treatment or therapy.

“Inhibiting UBE3A” or “inhibiting UBE3A” can mean reducing the level or expression of a UBE3A mRNA and/or UBE3A protein. In certain embodiments, UBE3A mRNA and/or UBE3A protein levels are inhibited in the presence of an antisense oligomer targeting UBE3A, including an antisense oligonucleotide targeting UBE3A, as compared to expression of UBE3A mRNA and/or UBE3A protein levels in the absence of a UBE3A antisense oligomer, such as an antisense oligonucleotide.

“Inhibiting the expression or activity” can refer to a reduction or blockade of the expression or activity and does not necessarily indicate a total elimination of expression or activity.

“Internucleoside linkage” can refer to the chemical bond between nucleosides.

“Intra-cisterna magna” or “ICM” injection or delivery can refer to injection of an agent or pharmaceutical composition provided herein in the cerebrospinal fluid (CSF)-filled subarachnoid space between the cerebellum and the dorsal side of the medulla oblongata.

“Linked nucleosides” can refer to adjacent nucleosides linked together by an internucleoside linkage.

“UBE3A antisense oligomer” can mean an antisense oligomer targeting UBE3A mRNA.

“Mismatch” or “non-complementary nucleobase” can refer to the case when a nucleobase of a first nucleic acid is not capable of pairing with the corresponding nucleobase of a second or target nucleic acid.

“Modified internucleoside linkage” can refer to a substitution or any change from a naturally occurring internucleoside bond (i.e., a phosphodiester internucleoside bond).

“Modified nucleobase” can refer to any nucleobase other than adenine, cytosine, guanine, thymidine, or uracil. An “unmodified nucleobase” means the purine bases adenine (A) and guanine (G), and the pyrimidine bases thymine (T), cytosine (C), and uracil (U).

“Modified nucleoside” can refer to a nucleoside having, independently, a modified sugar moiety and/or modified nucleobase.

“Modified nucleotide” can refer to a nucleotide having, independently, a modified sugar moiety, modified intemucleoside linkage, and/or modified nucleobase.

“Modified antisense oligonucleotide” can refer to an oligonucleotide comprising at least one modified internucleoside linkage, modified sugar, and/or modified nucleobase.

“Modified sugar” can refer to substitution and/or any change from a natural sugar moiety.

“Monomer” can refer to a single unit of an oligomer. Monomers include, but are not limited to, nucleosides and nucleotides, whether naturally occurring or modified. “Motif means the pattern of unmodified and modified nucleosides in an antisense oligomer.

“Natural sugar moiety” can refer to a sugar moiety found in DNA (2′-H) or RNA (2′-OH).

“Naturally occurring internucleoside linkage” can refer to a 3′ to 5′ phosphodiester linkage.

“Non-complementary nucleobase” can refer to a pair of nucleobases that do not form hydrogen bonds with one another or otherwise support hybridization.

“Nucleic acid” can refer to molecules composed of monomeric nucleotides. A nucleic acid includes, but is not limited to, ribonucleic acids (RNA), deoxyribonucleic acids (DNA), single-stranded nucleic acids, double-stranded nucleic acids, small interfering ribonucleic acids (siRNA), and microRNAs (miRNA).

“Nucleobase” can mean a heterocyclic moiety capable of pairing with a base of another nucleic acid. “Nucleobase complementarity” can refer to a nucleobase that is capable of base pairing with another nucleobase. For example, in DNA, adenine (A) is complementary to thymine (T). For example, in RNA, adenine (A) is complementary to uracil (U). In certain embodiments, complementary nucleobase refers to a nucleobase of an antisense oligomer that is capable of base pairing with a nucleobase of its target nucleic acid. For example, if a nucleobase at a certain position of an antisense oligomer is capable of hydrogen bonding with a nucleobase at a certain position of a target nucleic acid, then the position of hydrogen bonding between the oligonucleotide and the target nucleic acid is considered to be complementary at that nucleobase pair.

“Nucleobase sequence” can refer to the order of contiguous nucleobases independent of any sugar, linkage, and/or nucleobase modification.

“Nucleoside” can refer to a nucleobase linked to a sugar.

“Nucleoside mimetic” can include those structures used to replace the sugar or the sugar and the base and not necessarily the linkage at one or more positions of an oligomeric compound such as, for example, nucleoside mimetics having morpholino, cyclohexenyl, cyclohexyl, tetrahydropyranyl, bicyclo, or tricyclo sugar mimetics, e.g., non-furanose sugar units. Nucleotide mimetic includes those structures used to replace the nucleoside and the linkage at one or more positions of an oligomeric compound such as, for example, peptide nucleic acids or morpholinos (morpholinos linked by —N(H)—C(═O)—O— or other non-phosphodiester linkage). Sugar surrogate overlaps with the slightly broader term nucleoside mimetic but is intended to indicate replacement of the sugar unit (furanose ring) only. The tetrahydropyranyl rings provided herein are illustrative of an example of a sugar surrogate wherein the furanose sugar group has been replaced with a tetrahydropyranyl ring system. “Mimetic” can refer to groups that are substituted for a sugar, a nucleobase, and/or internucleoside linkage. Generally, a mimetic can be used in place of the sugar or sugar-internucleoside linkage combination, and the nucleobase is maintained for hybridization to a selected target.

“Nucleotide” can refer to a nucleoside having a phosphate group covalently linked to the sugar portion of the nucleoside.

“Oligomeric compound” or “oligomer,” which are used herein interchangeably, can refer to a polymer of linked monomeric subunits which is capable of hybridizing to at least a region of a nucleic acid molecule.

“Oligonucleotide” can refer to a polymer of linked nucleosides each of which can be modified or unmodified, independent one from another.

“Parenteral administration” can refer to administration through injection (e.g., bolus injection) or infusion. Parenteral administration can include subcutaneous administration, intravenous administration, intramuscular administration, intraarterial administration, intraperitoneal administration, or intracranial administration, e.g., intrathecal, intracerebroventricular, or intra cisterna magna administration.

“Peptide” can refer to a molecule formed by linking at least two amino acids by amide bonds. Without limitation, as used herein, peptide refers to polypeptides and proteins.

“Pharmaceutical agent” can refer to a substance that provides a therapeutic benefit when administered to an individual. For example, in certain embodiments, an antisense oligonucleotide targeted to UBE3A is a pharmaceutical agent.

“Pharmaceutical composition” can refer to a mixture of substances suitable for administering to an individual. For example, a pharmaceutical composition can comprise an antisense oligonucleotide and a sterile aqueous solution.

“Pharmaceutically acceptable salts” can refer to physiologically and pharmaceutically acceptable salts of a pharmaceutically active ingredient (e.g., an antisense oligomer provided herein), such as salts that retain the desired biological activity of the active ingredient and do not impart undesired toxicological effects thereto.

“Phosphorothioate linkage” can mean a linkage between nucleosides where the phosphodiester bond is modified by replacing one of the non-bridging oxygen atoms with a sulfur atom. A phosphorothioate linkage is a modified intemucleoside linkage.

“Portion” can mean a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an antisense oligomer.

“Prevent” or “preventing” can mean delaying or forestalling the onset or development of a disorder or syndrome for a period of time from minutes to days, weeks to months, or indefinitely.

“Prophylactically effective amount” can mean an amount of a pharmaceutical agent that provides a prophylactic or preventative benefit to an animal.

“Ribonucleotide” can mean a nucleotide having a hydroxy at the 2′ position of the sugar portion of the nucleotide. Ribonucleotides may be modified with any of a variety of substituents.

“Segments” are defined as smaller or sub-portions of regions within a target nucleic acid.

“Targeting” or “targeted” can mean the process of design and selection of an antisense oligomer that will specifically hybridize to a target nucleic acid and induce a desired effect.

“Target nucleic acid,” “target RNA,” and “target RNA transcript” and “nucleic acid target” all can mean a nucleic acid capable of being targeted by antisense oligomers. In certain embodiments, the target nucleic acid is a UBE2A nucleic acid.

“Target region” can mean a portion of a target nucleic acid to which one or more antisense oligomers is targeted.

“Target segment” can mean the sequence of nucleotides of a target nucleic acid to which an antisense oligomer is targeted. “5′ target site” refers to the 5′-most nucleotide of a target segment. “3′ target site” refers to the 3′-most nucleotide of a target segment.

“Therapeutically effective amount” can mean an amount of a pharmaceutical agent that provides a therapeutic benefit to an individual. “Treat” or “treating” or “treatment” can refer to administering a composition to effect an alteration or improvement of the disorder or syndrome.

“Unmodified nucleobases” can mean the purine bases adenine (A) and guanine (G), and the pyrimidine bases thymine (T), cytosine (C) and uracil (U).

“Unmodified nucleotide” can mean a nucleotide composed of naturally occurring nucleobases, sugar moieties, and internucleoside linkages. In certain embodiments, an unmodified nucleotide is an RNA nucleotide (i.e., β-D-ribonucleosides) or a DNA nucleotide (i.e., β-D-deoxyribonucleoside).

“Wing segment” can mean a plurality of nucleosides modified to impart to an oligonucleotide properties such as enhanced inhibitory activity, increased binding affinity for a target nucleic acid, or resistance to degradation by in vivo nucleases.

In general, the present disclosure relates to agents (e.g., antisense oligomers, e.g., antisense oligonucleotides), compositions, kits, and methods that relate to modulation of UBE3A level in a mammalian cell. In some cases, the agent provided herein modulates a level of a processed mRNA transcript encoding a UBE3A protein in a mammalian cell. In some cases, provided herein are agents, compositions, kits, and methods that relate to reduction of expression of a UBE3A protein in a mammalian cell where the UBE3A is overexpressed, for instance, in a mammalian cell having duplication, overexpression, or a gain-of-function mutation, of a UBE3A gene that encodes the UBE3A protein.

### Target Nucleic Acids, Target Regions and Nucleotide Sequences

Ubiquitin-protein ligase E3A (UBE3A), also known as E6AP ubiquitin-protein ligase (E6AP), is an enzyme involved in targeting proteins for degradation within cells. In human, UBE3A protein is encoded the UBE3A gene. The UBE3A gene is located on the long (q) arm of chromosome 15, 15q11.2, between positions 11 and 13. In cells, UBE3A protein can attach ubiquitin to proteins to be degraded by ubiquitin-proteasome degradation mechanism. Upon ubiquitin tagging, proteins can be recognized and digested by proteasomes. In humans, both copies of the UBE3A gene can be active in most of the body's tissues. However, in most neurons, only the maternal copy of UBE3A gene is normally active.

Nucleotide sequences that encode UBE3A include, without limitation, the complement of location 25333728 to 25439056 of GENBANK Accession No. NC_000015.10, or the complement of location 556325..658147 of GENBANK Accession No. NG_002690.1. Nucleotide sequences that encode UBE3A include, without limitation, those listed in Table 1. Nucleotide sequences that encode UBE3A include, without limitation, those any of the mRNA transcripts listed in Table 2. It is understood that the sequence set forth in each SEQ ID NO in Table 1 and Examples contained herein is independent of any modification to a sugar moiety, an internucleoside linkage, or a nucleobase. As such, antisense oligomers defined by a SEQ ID NO can comprise, independently, one or more modifications to a sugar moiety, an intemucleoside linkage, or a nucleobase.

In some cases, an agent (e.g., an antisense oligomer) provided herein comprises a polynucleotide sequence that is at least 80% complementary to at least 8 contiguous nucleic acids of the sequence set forth in any one of SEQ ID NO: 93-120. In some cases, an agent (e.g., an antisense oligomer) provided herein comprises a polynucleotide sequence that is at least 85%, 90%, 95%, 98%, or 100% complementary to at least 8 contiguous nucleic acids of the sequence set forth in any one of SEQ ID NO: 93-120. In some cases, an agent (e.g., an antisense oligomer) provided herein comprises a polynucleotide sequence that is 100% complementary to at least 8 contiguous nucleic acids of the sequence set forth in any one of SEQ ID NO: 93-120.

In some cases, an agent (e.g., an antisense oligomer) provided herein comprises a polynucleotide sequence that is at least 80% complementary to at least 8 contiguous nucleic acids of an mRNA transcript listed in Table 2. In some cases, an agent (e.g., an antisense oligomer) provided herein comprises a polynucleotide sequence that is at least 85%, 90%, 95%, 98%, or 100% complementary to at least 8 contiguous nucleic acids of an mRNA transcript listed in Table 2. In some cases, an agent (e.g., an antisense oligomer) provided herein comprises a polynucleotide sequence that is 100% complementary to at least 8 contiguous nucleic acids of an mRNA transcript listed in Table 2.

In certain embodiments, a target region is a structurally defined region of the target nucleic acid. For example, a target region can encompass a 3′ UTR, a 5′ UTR, an exon, an intron, an exon/intron junction, a coding region, a translation initiation region, translation termination region, or other defined nucleic acid region. The structurally defined regions for UBE3A can be obtained by accession number from sequence databases such as NCBI and such information is incorporated herein by reference. In certain embodiments, a target region encompasses the sequence from a 5′ target site of one target segment within the target region to a 3′ target site of another target segment within the same target region.

Targeting can include determination of at least one target segment to which an antisense oligomer hybridizes, such that a desired effect (e.g., degradation of the mRNA transcript that contains the at least one target segment) occurs. In certain embodiments, the desired effect is a reduction in mRNA target nucleic acid levels. In certain embodiments, the desired effect is reduction of levels of protein encoded by the target nucleic acid or a phenotypic change associated with the target nucleic acid.

A target region can contain one or more target segments. Multiple target segments within a target region can be overlapping. Alternatively, they can be non-overlapping. In certain embodiments, target segments within a target region are separated by no more than about 300 nucleotides. In certain embodiments, target segments within a target region are separated by a number of nucleotides that is, is about, is no more than, is no more than about, 250, 200, 150, 100, 90, 80, 70, 60, 50, 40, 30, 20, or 10 nucleotides on the target nucleic acid, or is a range defined by any two of the preceding values. In certain embodiments, target segments within a target region are separated by no more than, or no more than about, 5 nucleotides on the target nucleic acid. In certain embodiments, target segments are contiguous. Contemplated are target regions defined by a range having a starting nucleic acid that is any of the 5′ target sites or 3′ target sites listed herein.

In some embodiments, hybridization occurs between an antisense oligomer disclosed herein and a UBE3A nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized.

Methods of determining whether a sequence is specifically hybridizable to a target nucleic acid are well known in the art. In certain embodiments, the antisense oligomers provided herein are specifically hybridizable with a UBE3A nucleic acid.

TABLE 1
Certain UBE3A Nucleic Acid Sequences
that an Agent Provided Herein Targets

| GENBANK Accession No. | SEQ ID NO |
|---|---|
| NM_000462.5 | 93 |
| NM_001354505.1 | 94 |
| NM_001354506.2 | 95 |
| NM_001354507.2 | 96 |
| NM_001354508.2 | 97 |
| NM_001354509.2 | 98 |
| NM_001354511.2 | 99 |
| NM_001354512.2 | 100 |
| NM_001354513.2 | 101 |
| NM_001354523.2 | 102 |
| NM_001354526.1 | 103 |
| NM_001354538.2 | 104 |
| NM_001354539.2 | 105 |
| NM_001354540.2 | 106 |
| NM_001354541.2 | 107 |
| NM_001354542.2 | 108 |
| NM_001354543.2 | 109 |
| NM_001354544.2 | 110 |
| NM_001354545.2 | 111 |
| NM_001354546.2 | 112 |
| NM_001354547.2 | 113 |
| NM_001354548.2 | 114 |
| NM_001354549.2 | 115 |
| NM_001354550.2 | 116 |
| NM_001354551.2 | 117 |
| NM_001374461.1 | 118 |
| NM_130838.4 | 119 |
| NM_130839.5 | 120 |

TABLE 2
Certain UBE3A mRNA Transcripts that
an Agent Provided Herein Targets

| Ensembl Transcript ID* | SEQ ID NO. |
|---|---|
| ENST00000648336.2 | 121 |
| ENST00000638155.1 | 122 |
| ENST00000438097.6 | 123 |
| ENST00000638011.1 | 124 |
| ENST00000637886.1 | 125 |
| ENST00000650110.1 | 126 |
| ENST00000630424.2 | 127 |
| ENST00000649550.1 | 128 |
| ENST00000566215.5 | 129 |
| ENST00000428984.6 | 130 |
| ENST00000635914.1 | 131 |
| ENST00000636667.1 | 132 |
| ENST00000625778.3 | 133 |
| ENST00000675177.1 | 134 |
| ENST00000629252.2 | 135 |
| ENST00000630907.2 | 136 |
| ENST00000628267.2 | 137 |
| ENST00000628733.2 | 138 |
| ENST00000625681.2 | 139 |
| ENST00000604860.3 | 140 |
| ENST00000626176.2 | 141 |
| ENST00000629886.2 | 142 |
| ENST00000628890.1 | 143 |
| ENST00000626589.2 | 144 |
| ENST00000676452.1 | 145 |
| ENST00000631247.1 | 146 |
| ENST00000626793.2 | 147 |
| ENST00000627018.1 | 148 |
| ENST00000675593.1 | 149 |
| ENST00000675000.1 | 150 |
| ENST00000675038.1 | 151 |
| ENST00000630607.2 | 152 |
| ENST00000626068.2 | 153 |

*Sequences of the listed transcripts are retrievable by searching the Transcript ID on e!Ensembl (useast.ensembl.org/index.html).

### Antisense Oligomers

In some aspects, the agent provided herein is an antisense oligomer. Antisense oligomers provided herein can include, but are not limited to, oligonucleotides, oligonucleosides, oligonucleotide analogs, oligonucleotide mimetics, antisense oligomers, antisense oligonucleotides, and siRNAs.

In certain embodiments, an antisense oligomer has a nucleobase sequence that, when written in the 5′ to 3′ direction, comprises the reverse complement of the target segment of a target nucleic acid to which it is designed to target. In certain such embodiments, an antisense oligonucleotide has a nucleobase sequence that, when written in the 5′ to 3′ direction, comprises the reverse complement of the target segment of a target nucleic acid to which it is designed to target.

In some cases, an agent provided herein comprises an antisense oligomer, and the antisense oligomer comprises a sequence with at least 80% identity to the sequence set forth in any one of SEQ ID NO: 1-92. In some cases, an agent provided herein comprises an antisense oligomer, and the antisense oligomer comprises a sequence with at least 90% identity to the sequence set forth in any one of SEQ ID NO: 1-92. In some cases, the antisense oligomer comprises a sequence with 100% identity to the sequence set forth in any one of SEQ ID NO: 1-92. In some cases, the antisense oligomer consists of a sequence with at least 80% identity to the sequence set forth in any one of SEQ ID NO: 1-92. In some cases, the antisense oligomer consists of a sequence with at least 90% identity to the sequence set forth in any one of SEQ ID NO: 1-92. In some cases, the antisense oligomer consists of a sequence with 100% identity to the sequence set forth in any one of SEQ ID NO: 1-92.

In some cases, an agent provided herein comprises an antisense oligomer, and the antisense oligomer comprises the sequence set forth in any one of SEQ ID NO: 1-92, with 0 to 4 nucleic acid substitutions. In some cases, an agent provided herein comprises an antisense oligomer, and the antisense oligomer comprises the sequence set forth in any one of SEQ ID NO: 1-92, with 0 to 3 nucleic acid substitutions. In some cases, an agent provided herein comprises an antisense oligomer, and the antisense oligomer comprises the sequence set forth in any one of SEQ ID NO: 1-92, with 0 to 2 nucleic acid substitutions. In some cases, an agent provided herein comprises an antisense oligomer, and the antisense oligomer comprises the sequence set forth in any one of SEQ ID NO: 1-92, with 0 to 1 nucleic acid substitution.

In some cases, an antisense oligomer provided herein comprises a sequence with at least 90%, 92%, 95%, 98%, or 100% identity to the sequence of SEQ ID NO: 1. In some cases, an antisense oligomer provided herein comprises the sequence of SEQ ID NO: 1.

In some cases, an antisense oligomer provided herein comprises a sequence with at least 90%, 92%, 95%, 98%, or 100% identity to the sequence of SEQ ID NO: 2. In some cases, an antisense oligomer provided herein comprises the sequence of SEQ ID NO: 2.

In some cases, an antisense oligomer provided herein comprises a sequence with at least 90%, 92%, 95%, 98%, or 100% identity to the sequence of SEQ ID NO: 3. In some cases, an antisense oligomer provided herein comprises the sequence of SEQ ID NO: 3.

In some cases, an antisense oligomer provided herein comprises a sequence with at least 90%, 92%, 95%, 98%, or 100% identity to the sequence of SEQ ID NO: 4. In some cases, an antisense oligomer provided herein comprises the sequence of SEQ ID NO: 4.

In some cases, an antisense oligomer provided herein comprises a sequence with at least 90%, 92%, 95%, 98%, or 100% identity to the sequence of SEQ ID NO: 5. In some cases, an antisense oligomer provided herein comprises the sequence of SEQ ID NO: 5.

In some cases, an antisense oligomer provided herein comprises a sequence with at least 90%, 92%, 95%, 98%, or 100% identity to the sequence of SEQ ID NO: 6. In some cases, an antisense oligomer provided herein comprises the sequence of SEQ ID NO: 6.

In some cases, an antisense oligomer provided herein comprises a sequence with at least 90%, 92%, 95%, 98%, or 100% identity to the sequence of SEQ ID NO: 7. In some cases, an antisense oligomer provided herein comprises the sequence of SEQ ID NO: 7.

In some cases, an antisense oligomer provided herein comprises a sequence with at least 90%, 92%, 95%, 98%, or 100% identity to the sequence of SEQ ID NO: 8. In some cases, an antisense oligomer provided herein comprises the sequence of SEQ ID NO: 8.

In some cases, an antisense oligomer provided herein comprises a sequence with at least 90%, 92%, 95%, 98%, or 100% identity to the sequence of SEQ ID NO: 9. In some cases, an antisense oligomer provided herein comprises the sequence of SEQ ID NO: 9.

In some cases, an antisense oligomer provided herein comprises a sequence with at least 90%, 92%, 95%, 98%, or 100% identity to the sequence of SEQ ID NO: 10. In some cases, an antisense oligomer provided herein comprises the sequence of SEQ ID NO: 10.

In some cases, an antisense oligomer provided herein comprises a sequence with at least 90%, 92%, 95%, 98%, or 100% identity to the sequence of SEQ ID NO: 11. In some cases, an antisense oligomer provided herein comprises the sequence of SEQ ID NO: 11.

In some cases, an antisense oligomer provided herein comprises a sequence with at least 90%, 92%, 95%, 98%, or 100% identity to the sequence of SEQ ID NO: 12. In some cases, an antisense oligomer provided herein comprises the sequence of SEQ ID NO: 12.

In some cases, an antisense oligomer provided herein comprises a sequence with at least 90%, 92%, 95%, 98%, or 100% identity to the sequence of SEQ ID NO: 13. In some cases, an antisense oligomer provided herein comprises the sequence of SEQ ID NO: 13.

In some cases, an antisense oligomer provided herein comprises a sequence with at least 90%, 92%, 95%, 98%, or 100% identity to the sequence of SEQ ID NO: 14. In some cases, an antisense oligomer provided herein comprises the sequence of SEQ ID NO: 14.

In some cases, an antisense oligomer provided herein comprises a sequence with at least 90%, 92%, 95%, 98%, or 100% identity to the sequence of SEQ ID NO: 15. In some cases, an antisense oligomer provided herein comprises the sequence of SEQ ID NO: 15.

In some cases, an antisense oligomer provided herein comprises a sequence with at least 90%, 92%, 95%, 98%, or 100% identity to the sequence of SEQ ID NO: 16. In some cases, an antisense oligomer provided herein comprises the sequence of SEQ ID NO: 16.

In some cases, an antisense oligomer provided herein comprises a sequence with at least 90%, 92%, 95%, 98%, or 100% identity to the sequence of SEQ ID NO: 17. In some cases, an antisense oligomer provided herein comprises the sequence of SEQ ID NO: 17.

In some cases, an antisense oligomer provided herein comprises a sequence with at least 90%, 92%, 95%, 98%, or 100% identity to the sequence of SEQ ID NO: 18. In some cases, an antisense oligomer provided herein comprises the sequence of SEQ ID NO: 18.

In some cases, an antisense oligomer provided herein comprises a sequence with at least 90%, 92%, 95%, 98%, or 100% identity to the sequence of SEQ ID NO: 19. In some cases, an antisense oligomer provided herein comprises the sequence of SEQ ID NO: 19.

In some cases, an antisense oligomer provided herein comprises a sequence with at least 90%, 92%, 95%, 98%, or 100% identity to the sequence of SEQ ID NO: 20. In some cases, an antisense oligomer provided herein comprises the sequence of SEQ ID NO: 20.

In some cases, an antisense oligomer provided herein comprises a sequence with at least 90%, 92%, 95%, 98%, or 100% identity to the sequence of SEQ ID NO: 21. In some cases, an antisense oligomer provided herein comprises the sequence of SEQ ID NO: 21.

In some cases, an antisense oligomer provided herein comprises a sequence with at least 90%, 92%, 95%, 98%, or 100% identity to the sequence of SEQ ID NO: 22. In some cases, an antisense oligomer provided herein comprises the sequence of SEQ ID NO: 22.

In some cases, an antisense oligomer provided herein comprises a sequence with at least 90%, 92%, 95%, 98%, or 100% identity to the sequence of SEQ ID NO: 23. In some cases, an antisense oligomer provided herein comprises the sequence of SEQ ID NO: 23.

In some cases, an antisense oligomer provided herein comprises a sequence with at least 90%, 92%, 95%, 98%, or 100% identity to the sequence of SEQ ID NO: 24. In some cases, an antisense oligomer provided herein comprises the sequence of SEQ ID NO: 24.

In some cases, an antisense oligomer provided herein comprises a sequence with at least 90%, 92%, 95%, 98%, or 100% identity to the sequence of SEQ ID NO: 25. In some cases, an antisense oligomer provided herein comprises the sequence of SEQ ID NO: 25.

In some cases, an antisense oligomer provided herein comprises a sequence with at least 90%, 92%, 95%, 98%, or 100% identity to the sequence of SEQ ID NO: 26. In some cases, an antisense oligomer provided herein comprises the sequence of SEQ ID NO: 26.

In some cases, an antisense oligomer provided herein comprises a sequence with at least 90%, 92%, 95%, 98%, or 100% identity to the sequence of SEQ ID NO: 27. In some cases, an antisense oligomer provided herein comprises the sequence of SEQ ID NO: 27.

In some cases, an antisense oligomer provided herein comprises a sequence with at least 90%, 92%, 95%, 98%, or 100% identity to the sequence of SEQ ID NO: 28. In some cases, an antisense oligomer provided herein comprises the sequence of SEQ ID NO: 28.

In some cases, an antisense oligomer provided herein comprises a sequence with at least 90%, 92%, 95%, 98%, or 100% identity to the sequence of SEQ ID NO: 29. In some cases, an antisense oligomer provided herein comprises the sequence of SEQ ID NO: 29.

In some cases, an antisense oligomer provided herein comprises a sequence with at least 90%, 92%, 95%, 98%, or 100% identity to the sequence of SEQ ID NO: 30. In some cases, an antisense oligomer provided herein comprises the sequence of SEQ ID NO: 30.

In some cases, an antisense oligomer provided herein comprises a sequence with at least 90%, 92%, 95%, 98%, or 100% identity to the sequence of SEQ ID NO: 31. In some cases, an antisense oligomer provided herein comprises the sequence of SEQ ID NO: 31.

In some cases, an antisense oligomer provided herein comprises a sequence with at least 90%, 92%, 95%, 98%, or 100% identity to the sequence of SEQ ID NO: 32. In some cases, an antisense oligomer provided herein comprises the sequence of SEQ ID NO: 32.

In some cases, an antisense oligomer provided herein comprises a sequence with at least 90%, 92%, 95%, 98%, or 100% identity to the sequence of SEQ ID NO: 33. In some cases, an antisense oligomer provided herein comprises the sequence of SEQ ID NO: 33.

In some cases, an antisense oligomer provided herein comprises a sequence with at least 90%, 92%, 95%, 98%, or 100% identity to the sequence of SEQ ID NO: 34. In some cases, an antisense oligomer provided herein comprises the sequence of SEQ ID NO: 34.

In some cases, an antisense oligomer provided herein comprises a sequence with at least 90%, 92%, 95%, 98%, or 100% identity to the sequence of SEQ ID NO: 35. In some cases, an antisense oligomer provided herein comprises the sequence of SEQ ID NO: 35.

In some cases, an antisense oligomer provided herein comprises a sequence with at least 90%, 92%, 95%, 98%, or 100% identity to the sequence of SEQ ID NO: 36. In some cases, an antisense oligomer provided herein comprises the sequence of SEQ ID NO: 36.

In some cases, an antisense oligomer provided herein comprises a sequence with at least 90%, 92%, 95%, 98%, or 100% identity to the sequence of SEQ ID NO: 37. In some cases, an antisense oligomer provided herein comprises the sequence of SEQ ID NO: 37.

In some cases, an antisense oligomer provided herein comprises a sequence with at least 90%, 92%, 95%, 98%, or 100% identity to the sequence of SEQ ID NO: 38. In some cases, an antisense oligomer provided herein comprises the sequence of SEQ ID NO: 38.

In some cases, an antisense oligomer provided herein comprises a sequence with at least 90%, 92%, 95%, 98%, or 100% identity to the sequence of SEQ ID NO: 39. In some cases, an antisense oligomer provided herein comprises the sequence of SEQ ID NO: 39.

In some cases, an antisense oligomer provided herein comprises a sequence with at least 90%, 92%, 95%, 98%, or 100% identity to the sequence of SEQ ID NO: 40. In some cases, an antisense oligomer provided herein comprises the sequence of SEQ ID NO: 40.

In some cases, an antisense oligomer provided herein comprises a sequence with at least 90%, 92%, 95%, 98%, or 100% identity to the sequence of SEQ ID NO: 41. In some cases, an antisense oligomer provided herein comprises the sequence of SEQ ID NO: 41.

In some cases, an antisense oligomer provided herein comprises a sequence with at least 90%, 92%, 95%, 98%, or 100% identity to the sequence of SEQ ID NO: 42. In some cases, an antisense oligomer provided herein comprises the sequence of SEQ ID NO: 42.

In some cases, an antisense oligomer provided herein comprises a sequence with at least 90%, 92%, 95%, 98%, or 100% identity to the sequence of SEQ ID NO: 43. In some cases, an antisense oligomer provided herein comprises the sequence of SEQ ID NO: 43.

In some cases, an antisense oligomer provided herein comprises a sequence with at least 90%, 92%, 95%, 98%, or 100% identity to the sequence of SEQ ID NO: 44. In some cases, an antisense oligomer provided herein comprises the sequence of SEQ ID NO: 44.

In some cases, an antisense oligomer provided herein comprises a sequence with at least 90%, 92%, 95%, 98%, or 100% identity to the sequence of SEQ ID NO: 45. In some cases, an antisense oligomer provided herein comprises the sequence of SEQ ID NO: 45.

In some cases, an antisense oligomer provided herein comprises a sequence with at least 90%, 92%, 95%, 98%, or 100% identity to the sequence of SEQ ID NO: 46. In some cases, an antisense oligomer provided herein comprises the sequence of SEQ ID NO: 46.

In some cases, an antisense oligomer provided herein comprises a sequence with at least 90%, 92%, 95%, 98%, or 100% identity to the sequence of SEQ ID NO: 47. In some cases, an antisense oligomer provided herein comprises the sequence of SEQ ID NO: 47.

In some cases, an antisense oligomer provided herein comprises a sequence with at least 90%, 92%, 95%, 98%, or 100% identity to the sequence of SEQ ID NO: 48. In some cases, an antisense oligomer provided herein comprises the sequence of SEQ ID NO: 48.

In some cases, an antisense oligomer provided herein comprises a sequence with at least 90%, 92%, 95%, 98%, or 100% identity to the sequence of SEQ ID NO: 49. In some cases, an antisense oligomer provided herein comprises the sequence of SEQ ID NO: 49.

In some cases, an antisense oligomer provided herein comprises a sequence with at least 90%, 92%, 95%, 98%, or 100% identity to the sequence of SEQ ID NO: 50. In some cases, an antisense oligomer provided herein comprises the sequence of SEQ ID NO: 50.

In some cases, an antisense oligomer provided herein comprises a sequence with at least 90%, 92%, 95%, 98%, or 100% identity to the sequence of SEQ ID NO: 51. In some cases, an antisense oligomer provided herein comprises the sequence of SEQ ID NO: 51.

In some cases, an antisense oligomer provided herein comprises a sequence with at least 90%, 92%, 95%, 98%, or 100% identity to the sequence of SEQ ID NO: 52. In some cases, an antisense oligomer provided herein comprises the sequence of SEQ ID NO: 52.

In some cases, an antisense oligomer provided herein comprises a sequence with at least 90%, 92%, 95%, 98%, or 100% identity to the sequence of SEQ ID NO: 53. In some cases, an antisense oligomer provided herein comprises the sequence of SEQ ID NO: 53.

In some cases, an antisense oligomer provided herein comprises a sequence with at least 90%, 92%, 95%, 98%, or 100% identity to the sequence of SEQ ID NO: 54. In some cases, an antisense oligomer provided herein comprises the sequence of SEQ ID NO: 54.

In some cases, an antisense oligomer provided herein comprises a sequence with at least 90%, 92%, 95%, 98%, or 100% identity to the sequence of SEQ ID NO: 55. In some cases, an antisense oligomer provided herein comprises the sequence of SEQ ID NO: 55.

In some cases, an antisense oligomer provided herein comprises a sequence with at least 90%, 92%, 95%, 98%, or 100% identity to the sequence of SEQ ID NO: 56. In some cases, an antisense oligomer provided herein comprises the sequence of SEQ ID NO: 56.

In some cases, an antisense oligomer provided herein comprises a sequence with at least 90%, 92%, 95%, 98%, or 100% identity to the sequence of SEQ ID NO: 57. In some cases, an antisense oligomer provided herein comprises the sequence of SEQ ID NO: 57.

In some cases, an antisense oligomer provided herein comprises a sequence with at least 90%, 92%, 95%, 98%, or 100% identity to the sequence of SEQ ID NO: 58. In some cases, an antisense oligomer provided herein comprises the sequence of SEQ ID NO: 58.

In some cases, an antisense oligomer provided herein comprises a sequence with at least 90%, 92%, 95%, 98%, or 100% identity to the sequence of SEQ ID NO: 59. In some cases, an antisense oligomer provided herein comprises the sequence of SEQ ID NO: 59.

In some cases, an antisense oligomer provided herein comprises a sequence with at least 90%, 92%, 95%, 98%, or 100% identity to the sequence of SEQ ID NO: 60. In some cases, an antisense oligomer provided herein comprises the sequence of SEQ ID NO: 60.

In some cases, an antisense oligomer provided herein comprises a sequence with at least 90%, 92%, 95%, 98%, or 100% identity to the sequence of SEQ ID NO: 61. In some cases, an antisense oligomer provided herein comprises the sequence of SEQ ID NO: 61.

In some cases, an antisense oligomer provided herein comprises a sequence with at least 90%, 92%, 95%, 98%, or 100% identity to the sequence of SEQ ID NO: 62. In some cases, an antisense oligomer provided herein comprises the sequence of SEQ ID NO: 62.

In some cases, an antisense oligomer provided herein comprises a sequence with at least 90%, 92%, 95%, 98%, or 100% identity to the sequence of SEQ ID NO: 63. In some cases, an antisense oligomer provided herein comprises the sequence of SEQ ID NO: 63.

In some cases, an antisense oligomer provided herein comprises a sequence with at least 90%, 92%, 95%, 98%, or 100% identity to the sequence of SEQ ID NO: 64. In some cases, an antisense oligomer provided herein comprises the sequence of SEQ ID NO: 64.

In some cases, an antisense oligomer provided herein comprises a sequence with at least 90%, 92%, 95%, 98%, or 100% identity to the sequence of SEQ ID NO: 65. In some cases, an antisense oligomer provided herein comprises the sequence of SEQ ID NO: 65.

In some cases, an antisense oligomer provided herein comprises a sequence with at least 90%, 92%, 95%, 98%, or 100% identity to the sequence of SEQ ID NO: 66. In some cases, an antisense oligomer provided herein comprises the sequence of SEQ ID NO: 66.

In some cases, an antisense oligomer provided herein comprises a sequence with at least 90%, 92%, 95%, 98%, or 100% identity to the sequence of SEQ ID NO: 67. In some cases, an antisense oligomer provided herein comprises the sequence of SEQ ID NO: 67.

In some cases, an antisense oligomer provided herein comprises a sequence with at least 90%, 92%, 95%, 98%, or 100% identity to the sequence of SEQ ID NO: 68. In some cases, an antisense oligomer provided herein comprises the sequence of SEQ ID NO: 68.

In some cases, an antisense oligomer provided herein comprises a sequence with at least 90%, 92%, 95%, 98%, or 100% identity to the sequence of SEQ ID NO: 69. In some cases, an antisense oligomer provided herein comprises the sequence of SEQ ID NO: 69.

In some cases, an antisense oligomer provided herein comprises a sequence with at least 90%, 92%, 95%, 98%, or 100% identity to the sequence of SEQ ID NO: 70. In some cases, an antisense oligomer provided herein comprises the sequence of SEQ ID NO: 70.

In some cases, an antisense oligomer provided herein comprises a sequence with at least 90%, 92%, 95%, 98%, or 100% identity to the sequence of SEQ ID NO: 71. In some cases, an antisense oligomer provided herein comprises the sequence of SEQ ID NO: 71.

In some cases, an antisense oligomer provided herein comprises a sequence with at least 90%, 92%, 95%, 98%, or 100% identity to the sequence of SEQ ID NO: 72. In some cases, an antisense oligomer provided herein comprises the sequence of SEQ ID NO: 72.

In some cases, an antisense oligomer provided herein comprises a sequence with at least 90%, 92%, 95%, 98%, or 100% identity to the sequence of SEQ ID NO: 73. In some cases, an antisense oligomer provided herein comprises the sequence of SEQ ID NO: 73.

In some cases, an antisense oligomer provided herein comprises a sequence with at least 90%, 92%, 95%, 98%, or 100% identity to the sequence of SEQ ID NO: 74. In some cases, an antisense oligomer provided herein comprises the sequence of SEQ ID NO: 74.

In some cases, an antisense oligomer provided herein comprises a sequence with at least 90%, 92%, 95%, 98%, or 100% identity to the sequence of SEQ ID NO: 75. In some cases, an antisense oligomer provided herein comprises the sequence of SEQ ID NO: 75.

In some cases, an antisense oligomer provided herein comprises a sequence with at least 90%, 92%, 95%, 98%, or 100% identity to the sequence of SEQ ID NO: 76. In some cases, an antisense oligomer provided herein comprises the sequence of SEQ ID NO: 76.

In some cases, an antisense oligomer provided herein comprises a sequence with at least 90%, 92%, 95%, 98%, or 100% identity to the sequence of SEQ ID NO: 77. In some cases, an antisense oligomer provided herein comprises the sequence of SEQ ID NO: 77.

In some cases, an antisense oligomer provided herein comprises a sequence with at least 90%, 92%, 95%, 98%, or 100% identity to the sequence of SEQ ID NO: 78. In some cases, an antisense oligomer provided herein comprises the sequence of SEQ ID NO: 78.

In some cases, an antisense oligomer provided herein comprises a sequence with at least 90%, 92%, 95%, 98%, or 100% identity to the sequence of SEQ ID NO: 79. In some cases, an antisense oligomer provided herein comprises the sequence of SEQ ID NO: 79.

In some cases, an antisense oligomer provided herein comprises a sequence with at least 90%, 92%, 95%, 98%, or 100% identity to the sequence of SEQ ID NO: 80. In some cases, an antisense oligomer provided herein comprises the sequence of SEQ ID NO: 80.

In some cases, an antisense oligomer provided herein comprises a sequence with at least 90%, 92%, 95%, 98%, or 100% identity to the sequence of SEQ ID NO: 81. In some cases, an antisense oligomer provided herein comprises the sequence of SEQ ID NO: 81.

In some cases, an antisense oligomer provided herein comprises a sequence with at least 90%, 92%, 95%, 98%, or 100% identity to the sequence of SEQ ID NO: 82. In some cases, an antisense oligomer provided herein comprises the sequence of SEQ ID NO: 82.

In some cases, an antisense oligomer provided herein comprises a sequence with at least 90%, 92%, 95%, 98%, or 100% identity to the sequence of SEQ ID NO: 83. In some cases, an antisense oligomer provided herein comprises the sequence of SEQ ID NO: 83.

In some cases, an antisense oligomer provided herein comprises a sequence with at least 90%, 92%, 95%, 98%, or 100% identity to the sequence of SEQ ID NO: 84. In some cases, an antisense oligomer provided herein comprises the sequence of SEQ ID NO: 84.

In some cases, an antisense oligomer provided herein comprises a sequence with at least 90%, 92%, 95%, 98%, or 100% identity to the sequence of SEQ ID NO: 85. In some cases, an antisense oligomer provided herein comprises the sequence of SEQ ID NO: 85.

In some cases, an antisense oligomer provided herein comprises a sequence with at least 90%, 92%, 95%, 98%, or 100% identity to the sequence of SEQ ID NO: 86. In some cases, an antisense oligomer provided herein comprises the sequence of SEQ ID NO: 86.

In some cases, an antisense oligomer provided herein comprises a sequence with at least 90%, 92%, 95%, 98%, or 100% identity to the sequence of SEQ ID NO: 87. In some cases, an antisense oligomer provided herein comprises the sequence of SEQ ID NO: 87.

In some cases, an antisense oligomer provided herein comprises a sequence with at least 90%, 92%, 95%, 98%, or 100% identity to the sequence of SEQ ID NO: 88. In some cases, an antisense oligomer provided herein comprises the sequence of SEQ ID NO: 88.

In some cases, an antisense oligomer provided herein comprises a sequence with at least 90%, 92%, 95%, 98%, or 100% identity to the sequence of SEQ ID NO: 89. In some cases, an antisense oligomer provided herein comprises the sequence of SEQ ID NO: 89.

In some cases, an antisense oligomer provided herein comprises a sequence with at least 90%, 92%, 95%, 98%, or 100% identity to the sequence of SEQ ID NO: 90. In some cases, an antisense oligomer provided herein comprises the sequence of SEQ ID NO: 90.

In some cases, an antisense oligomer provided herein comprises a sequence with at least 90%, 92%, 95%, 98%, or 100% identity to the sequence of SEQ ID NO: 91. In some cases, an antisense oligomer provided herein comprises the sequence of SEQ ID NO: 91.

In some cases, an antisense oligomer provided herein comprises a sequence with at least 90%, 92%, 95%, 98%, or 100% identity to the sequence of SEQ ID NO: 92. In some cases, an antisense oligomer provided herein comprises the sequence of SEQ ID NO: 92.

In some cases, an antisense oligomer provided herein comprises the sequence of SEQ ID NO: 1, with 0 to 4 nucleic acid substitutions. In some cases, an antisense oligomer provided herein comprises the sequence of SEQ ID NO: 1, with 0 to 3 nucleic acid substitutions. In some cases, an antisense oligomer provided herein comprises the sequence of SEQ ID NO: 1, with 0 to 2 nucleic acid substitutions. In some cases, an antisense oligomer provided herein comprises the sequence of SEQ ID NO: 1, with 0 to 1 nucleic acid substitution.

In some cases, an antisense oligomer provided herein comprises the sequence of SEQ ID NO: 2, with 0 to 4 nucleic acid substitutions. In some cases, an antisense oligomer provided herein comprises the sequence of SEQ ID NO: 2, with 0 to 3 nucleic acid substitutions. In some cases, an antisense oligomer provided herein comprises the sequence of SEQ ID NO: 2, with 0 to 2 nucleic acid substitutions. In some cases, an antisense oligomer provided herein comprises the sequence of SEQ ID NO: 2, with 0 to 1 nucleic acid substitution.

In some cases, an antisense oligomer provided herein comprises the sequence of SEQ ID NO: 3, with 0 to 4 nucleic acid substitutions. In some cases, an antisense oligomer provided herein comprises the sequence of SEQ ID NO: 3, with 0 to 3 nucleic acid substitutions. In some cases, an antisense oligomer provided herein comprises the sequence of SEQ ID NO: 3, with 0 to 2 nucleic acid substitutions. In some cases, an antisense oligomer provided herein comprises the sequence of SEQ ID NO: 3, with 0 to 1 nucleic acid substitution.

In some cases, an antisense oligomer provided herein comprises the sequence of SEQ ID NO: 4, with 0 to 4 nucleic acid substitutions. In some cases, an antisense oligomer provided herein comprises the sequence of SEQ ID NO: 4, with 0 to 3 nucleic acid substitutions. In some cases, an antisense oligomer provided herein comprises the sequence of SEQ ID NO: 4, with 0 to 2 nucleic acid substitutions. In some cases, an antisense oligomer provided herein comprises the sequence of SEQ ID NO: 4, with 0 to 1 nucleic acid substitution.

In some cases, an antisense oligomer provided herein comprises the sequence of SEQ ID NO: 5, with 0 to 4 nucleic acid substitutions. In some cases, an antisense oligomer provided herein comprises the sequence of SEQ ID NO: 5, with 0 to 3 nucleic acid substitutions. In some cases, an antisense oligomer provided herein comprises the sequence of SEQ ID NO: 5, with 0 to 2 nucleic acid substitutions. In some cases, an antisense oligomer provided herein comprises the sequence of SEQ ID NO: 5, with 0 to 1 nucleic acid substitution.

In some cases, an antisense oligomer provided herein comprises the sequence of SEQ ID NO: 6, with 0 to 4 nucleic acid substitutions. In some cases, an antisense oligomer provided herein comprises the sequence of SEQ ID NO: 6, with 0 to 3 nucleic acid substitutions. In some cases, an antisense oligomer provided herein comprises the sequence of SEQ ID NO: 6, with 0 to 2 nucleic acid substitutions. In some cases, an antisense oligomer provided herein comprises the sequence of SEQ ID NO: 6, with 0 to 1 nucleic acid substitution.

In some cases, an antisense oligomer provided herein comprises the sequence of SEQ ID NO: 7, with 0 to 4 nucleic acid substitutions. In some cases, an antisense oligomer provided herein comprises the sequence of SEQ ID NO: 7, with 0 to 3 nucleic acid substitutions. In some cases, an antisense oligomer provided herein comprises the sequence of SEQ ID NO: 7, with 0 to 2 nucleic acid substitutions. In some cases, an antisense oligomer provided herein comprises the sequence of SEQ ID NO: 7, with 0 to 1 nucleic acid substitution.

In some cases, an antisense oligomer provided herein comprises the sequence of SEQ ID NO: 8, with 0 to 4 nucleic acid substitutions. In some cases, an antisense oligomer provided herein comprises the sequence of SEQ ID NO: 8, with 0 to 3 nucleic acid substitutions. In some cases, an antisense oligomer provided herein comprises the sequence of SEQ ID NO: 8, with 0 to 2 nucleic acid substitutions. In some cases, an antisense oligomer provided herein comprises the sequence of SEQ ID NO: 8, with 0 to 1 nucleic acid substitution.

In some cases, an antisense oligomer provided herein comprises the sequence of SEQ ID NO: 9, with 0 to 4 nucleic acid substitutions. In some cases, an antisense oligomer provided herein comprises the sequence of SEQ ID NO: 9, with 0 to 3 nucleic acid substitutions. In some cases, an antisense oligomer provided herein comprises the sequence of SEQ ID NO: 9, with 0 to 2 nucleic acid substitutions. In some cases, an antisense oligomer provided herein comprises the sequence of SEQ ID NO: 9, with 0 to 1 nucleic acid substitution.

In some cases, an antisense oligomer provided herein comprises the sequence of SEQ ID NO: 10, with 0 to 4 nucleic acid substitutions. In some cases, an antisense oligomer provided herein comprises the sequence of SEQ ID NO: 10, with 0 to 3 nucleic acid substitutions. In some cases, an antisense oligomer provided herein comprises the sequence of SEQ ID NO: 10, with 0 to 2 nucleic acid substitutions. In some cases, an antisense oligomer provided herein comprises the sequence of SEQ ID NO: 10, with 0 to 1 nucleic acid substitution.

In some cases, an antisense oligomer provided herein comprises the sequence of SEQ ID NO: 11, with 0 to 4 nucleic acid substitutions. In some cases, an antisense oligomer provided herein comprises the sequence of SEQ ID NO: 11, with 0 to 3 nucleic acid substitutions. In some cases, an antisense oligomer provided herein comprises the sequence of SEQ ID NO: 11, with 0 to 2 nucleic acid substitutions. In some cases, an antisense oligomer provided herein comprises the sequence of SEQ ID NO: 11, with 0 to 1 nucleic acid substitution.

In some cases, an antisense oligomer provided herein comprises the sequence of SEQ ID NO: 12, with 0 to 4 nucleic acid substitutions. In some cases, an antisense oligomer provided herein comprises the sequence of SEQ ID NO: 12, with 0 to 3 nucleic acid substitutions. In some cases, an antisense oligomer provided herein comprises the sequence of SEQ ID NO: 12, with 0 to 2 nucleic acid substitutions. In some cases, an antisense oligomer provided herein comprises the sequence of SEQ ID NO: 12, with 0 to 1 nucleic acid substitution.

In some cases, an antisense oligomer provided herein comprises the sequence of SEQ ID NO: 13, with 0 to 4 nucleic acid substitutions. In some cases, an antisense oligomer provided herein comprises the sequence of SEQ ID NO: 13, with 0 to 3 nucleic acid substitutions. In some cases, an antisense oligomer provided herein comprises the sequence of SEQ ID NO: 13, with 0 to 2 nucleic acid substitutions. In some cases, an antisense oligomer provided herein comprises the sequence of SEQ ID NO: 13, with 0 to 1 nucleic acid substitution.

In some cases, an antisense oligomer provided herein comprises the sequence of SEQ ID NO: 14, with 0 to 4 nucleic acid substitutions. In some cases, an antisense oligomer provided herein comprises the sequence of SEQ ID NO: 14, with 0 to 3 nucleic acid substitutions. In some cases, an antisense oligomer provided herein comprises the sequence of SEQ ID NO: 14, with 0 to 2 nucleic acid substitutions. In some cases, an antisense oligomer provided herein comprises the sequence of SEQ ID NO: 14, with 0 to 1 nucleic acid substitution.

In some cases, an antisense oligomer provided herein comprises the sequence of SEQ ID NO: 15, with 0 to 4 nucleic acid substitutions. In some cases, an antisense oligomer provided herein comprises the sequence of SEQ ID NO: 15, with 0 to 3 nucleic acid substitutions. In some cases, an antisense oligomer provided herein comprises the sequence of SEQ ID NO: 15, with 0 to 2 nucleic acid substitutions. In some cases, an antisense oligomer provided herein comprises the sequence of SEQ ID NO: 15, with 0 to 1 nucleic acid substitution.

In some cases, an antisense oligomer provided herein comprises the sequence of SEQ ID NO: 16, with 0 to 4 nucleic acid substitutions. In some cases, an antisense oligomer provided herein comprises the sequence of SEQ ID NO: 16, with 0 to 3 nucleic acid substitutions. In some cases, an antisense oligomer provided herein comprises the sequence of SEQ ID NO: 16, with 0 to 2 nucleic acid substitutions. In some cases, an antisense oligomer provided herein comprises the sequence of SEQ ID NO: 16, with 0 to 1 nucleic acid substitution.

In some cases, an antisense oligomer provided herein comprises the sequence of SEQ ID NO: 17, with 0 to 4 nucleic acid substitutions. In some cases, an antisense oligomer provided herein comprises the sequence of SEQ ID NO: 17, with 0 to 3 nucleic acid substitutions. In some cases, an antisense oligomer provided herein comprises the sequence of SEQ ID NO: 17, with 0 to 2 nucleic acid substitutions. In some cases, an antisense oligomer provided herein comprises the sequence of SEQ ID NO: 17, with 0 to 1 nucleic acid substitution.

In some cases, an antisense oligomer provided herein comprises the sequence of SEQ ID NO: 18, with 0 to 4 nucleic acid substitutions. In some cases, an antisense oligomer provided herein comprises the sequence of SEQ ID NO: 18, with 0 to 3 nucleic acid substitutions. In some cases, an antisense oligomer provided herein comprises the sequence of SEQ ID NO: 18, with 0 to 2 nucleic acid substitutions. In some cases, an antisense oligomer provided herein comprises the sequence of SEQ ID NO: 18, with 0 to 1 nucleic acid substitution.

In some cases, an antisense oligomer provided herein comprises the sequence of SEQ ID NO: 19, with 0 to 4 nucleic acid substitutions. In some cases, an antisense oligomer provided herein comprises the sequence of SEQ ID NO: 19, with 0 to 3 nucleic acid substitutions. In some cases, an antisense oligomer provided herein comprises the sequence of SEQ ID NO: 19, with 0 to 2 nucleic acid substitutions. In some cases, an antisense oligomer provided herein comprises the sequence of SEQ ID NO: 19, with 0 to 1 nucleic acid substitution.

In some cases, an antisense oligomer provided herein comprises the sequence of SEQ ID NO: 20, with 0 to 4 nucleic acid substitutions. In some cases, an antisense oligomer provided herein comprises the sequence of SEQ ID NO: 20, with 0 to 3 nucleic acid substitutions. In some cases, an antisense oligomer provided herein comprises the sequence of SEQ ID NO: 20, with 0 to 2 nucleic acid substitutions. In some cases, an antisense oligomer provided herein comprises the sequence of SEQ ID NO: 20, with 0 to 1 nucleic acid substitution.

In some cases, an antisense oligomer provided herein comprises the sequence of SEQ ID NO: 21, with 0 to 4 nucleic acid substitutions. In some cases, an antisense oligomer provided herein comprises the sequence of SEQ ID NO: 21, with 0 to 3 nucleic acid substitutions. In some cases, an antisense oligomer provided herein comprises the sequence of SEQ ID NO: 21, with 0 to 2 nucleic acid substitutions. In some cases, an antisense oligomer provided herein comprises the sequence of SEQ ID NO: 21, with 0 to 1 nucleic acid substitution.

In some cases, an antisense oligomer provided herein comprises the sequence of SEQ ID NO: 22, with 0 to 4 nucleic acid substitutions. In some cases, an antisense oligomer provided herein comprises the sequence of SEQ ID NO: 22, with 0 to 3 nucleic acid substitutions. In some cases, an antisense oligomer provided herein comprises the sequence of SEQ ID NO: 22, with 0 to 2 nucleic acid substitutions. In some cases, an antisense oligomer provided herein comprises the sequence of SEQ ID NO: 22, with 0 to 1 nucleic acid substitution.

In some cases, an antisense oligomer provided herein comprises the sequence of SEQ ID NO: 23, with 0 to 4 nucleic acid substitutions. In some cases, an antisense oligomer provided herein comprises the sequence of SEQ ID NO: 23, with 0 to 3 nucleic acid substitutions. In some cases, an antisense oligomer provided herein comprises the sequence of SEQ ID NO: 23, with 0 to 2 nucleic acid substitutions. In some cases, an antisense oligomer provided herein comprises the sequence of SEQ ID NO: 23, with 0 to 1 nucleic acid substitution.

In some cases, an antisense oligomer provided herein comprises the sequence of SEQ ID NO: 24, with 0 to 4 nucleic acid substitutions. In some cases, an antisense oligomer provided herein comprises the sequence of SEQ ID NO: 24, with 0 to 3 nucleic acid substitutions. In some cases, an antisense oligomer provided herein comprises the sequence of SEQ ID NO: 24, with 0 to 2 nucleic acid substitutions. In some cases, an antisense oligomer provided herein comprises the sequence of SEQ ID NO: 24, with 0 to 1 nucleic acid substitution.

In some cases, an antisense oligomer provided herein comprises the sequence of SEQ ID NO: 25, with 0 to 4 nucleic acid substitutions. In some cases, an antisense oligomer provided herein comprises the sequence of SEQ ID NO: 25, with 0 to 3 nucleic acid substitutions. In some cases, an antisense oligomer provided herein comprises the sequence of SEQ ID NO: 25, with 0 to 2 nucleic acid substitutions. In some cases, an antisense oligomer provided herein comprises the sequence of SEQ ID NO: 25, with 0 to 1 nucleic acid substitution.

In some cases, an antisense oligomer provided herein comprises the sequence of SEQ ID NO: 26, with 0 to 4 nucleic acid substitutions. In some cases, an antisense oligomer provided herein comprises the sequence of SEQ ID NO: 26, with 0 to 3 nucleic acid substitutions. In some cases, an antisense oligomer provided herein comprises the sequence of SEQ ID NO: 26, with 0 to 2 nucleic acid substitutions. In some cases, an antisense oligomer provided herein comprises the sequence of SEQ ID NO: 26, with 0 to 1 nucleic acid substitution.

In some cases, an antisense oligomer provided herein comprises the sequence of SEQ ID NO: 27, with 0 to 4 nucleic acid substitutions. In some cases, an antisense oligomer provided herein comprises the sequence of SEQ ID NO: 27, with 0 to 3 nucleic acid substitutions. In some cases, an antisense oligomer provided herein comprises the sequence of SEQ ID NO: 27, with 0 to 2 nucleic acid substitutions. In some cases, an antisense oligomer provided herein comprises the sequence of SEQ ID NO: 27, with 0 to 1 nucleic acid substitution.

In some cases, an antisense oligomer provided herein comprises the sequence of SEQ ID NO: 28, with 0 to 4 nucleic acid substitutions. In some cases, an antisense oligomer provided herein comprises the sequence of SEQ ID NO: 28, with 0 to 3 nucleic acid substitutions. In some cases, an antisense oligomer provided herein comprises the sequence of SEQ ID NO: 28, with 0 to 2 nucleic acid substitutions. In some cases, an antisense oligomer provided herein comprises the sequence of SEQ ID NO: 28, with 0 to 1 nucleic acid substitution.

In some cases, an antisense oligomer provided herein comprises the sequence of SEQ ID NO: 29, with 0 to 4 nucleic acid substitutions. In some cases, an antisense oligomer provided herein comprises the sequence of SEQ ID NO: 29, with 0 to 3 nucleic acid substitutions. In some cases, an antisense oligomer provided herein comprises the sequence of SEQ ID NO: 29, with 0 to 2 nucleic acid substitutions. In some cases, an antisense oligomer provided herein comprises the sequence of SEQ ID NO: 29, with 0 to 1 nucleic acid substitution.

In some cases, an antisense oligomer provided herein comprises the sequence of SEQ ID NO: 30, with 0 to 4 nucleic acid substitutions. In some cases, an antisense oligomer provided herein comprises the sequence of SEQ ID NO: 30, with 0 to 3 nucleic acid substitutions. In some cases, an antisense oligomer provided herein comprises the sequence of SEQ ID NO: 30, with 0 to 2 nucleic acid substitutions. In some cases, an antisense oligomer provided herein comprises the sequence of SEQ ID NO: 30, with 0 to 1 nucleic acid substitution.

In some cases, an antisense oligomer provided herein comprises the sequence of SEQ ID NO: 31, with 0 to 4 nucleic acid substitutions. In some cases, an antisense oligomer provided herein comprises the sequence of SEQ ID NO: 31, with 0 to 3 nucleic acid substitutions. In some cases, an antisense oligomer provided herein comprises the sequence of SEQ ID NO: 31, with 0 to 2 nucleic acid substitutions. In some cases, an antisense oligomer provided herein comprises the sequence of SEQ ID NO: 31, with 0 to 1 nucleic acid substitution.

In some cases, an antisense oligomer provided herein comprises the sequence of SEQ ID NO: 32, with 0 to 4 nucleic acid substitutions. In some cases, an antisense oligomer provided herein comprises the sequence of SEQ ID NO: 32, with 0 to 3 nucleic acid substitutions. In some cases, an antisense oligomer provided herein comprises the sequence of SEQ ID NO: 32, with 0 to 2 nucleic acid substitutions. In some cases, an antisense oligomer provided herein comprises the sequence of SEQ ID NO: 32, with 0 to 1 nucleic acid substitution.

In some cases, an antisense oligomer provided herein comprises the sequence of SEQ ID NO: 33, with 0 to 4 nucleic acid substitutions. In some cases, an antisense oligomer provided herein comprises the sequence of SEQ ID NO: 33, with 0 to 3 nucleic acid substitutions. In some cases, an antisense oligomer provided herein comprises the sequence of SEQ ID NO: 33, with 0 to 2 nucleic acid substitutions. In some cases, an antisense oligomer provided herein comprises the sequence of SEQ ID NO: 33, with 0 to 1 nucleic acid substitution.

In some cases, an antisense oligomer provided herein comprises the sequence of SEQ ID NO: 34, with 0 to 4 nucleic acid substitutions. In some cases, an antisense oligomer provided herein comprises the sequence of SEQ ID NO: 34, with 0 to 3 nucleic acid substitutions. In some cases, an antisense oligomer provided herein comprises the sequence of SEQ ID NO: 34, with 0 to 2 nucleic acid substitutions. In some cases, an antisense oligomer provided herein comprises the sequence of SEQ ID NO: 34, with 0 to 1 nucleic acid substitution.

In some cases, an antisense oligomer provided herein comprises the sequence of SEQ ID NO: 35, with 0 to 4 nucleic acid substitutions. In some cases, an antisense oligomer provided herein comprises the sequence of SEQ ID NO: 35, with 0 to 3 nucleic acid substitutions. In some cases, an antisense oligomer provided herein comprises the sequence of SEQ ID NO: 35, with 0 to 2 nucleic acid substitutions. In some cases, an antisense oligomer provided herein comprises the sequence of SEQ ID NO: 35, with 0 to 1 nucleic acid substitution.

In some cases, an antisense oligomer provided herein comprises the sequence of SEQ ID NO: 36, with 0 to 4 nucleic acid substitutions. In some cases, an antisense oligomer provided herein comprises the sequence of SEQ ID NO: 36, with 0 to 3 nucleic acid substitutions. In some cases, an antisense oligomer provided herein comprises the sequence of SEQ ID NO: 36, with 0 to 2 nucleic acid substitutions. In some cases, an antisense oligomer provided herein comprises the sequence of SEQ ID NO: 36, with 0 to 1 nucleic acid substitution.

In some cases, an antisense oligomer provided herein comprises the sequence of SEQ ID NO: 37, with 0 to 4 nucleic acid substitutions. In some cases, an antisense oligomer provided herein comprises the sequence of SEQ ID NO: 37, with 0 to 3 nucleic acid substitutions. In some cases, an antisense oligomer provided herein comprises the sequence of SEQ ID NO: 37, with 0 to 2 nucleic acid substitutions. In some cases, an antisense oligomer provided herein comprises the sequence of SEQ ID NO: 37, with 0 to 1 nucleic acid substitution.

In some cases, an antisense oligomer provided herein comprises the sequence of SEQ ID NO: 38, with 0 to 4 nucleic acid substitutions. In some cases, an antisense oligomer provided herein comprises the sequence of SEQ ID NO: 38, with 0 to 3 nucleic acid substitutions. In some cases, an antisense oligomer provided herein comprises the sequence of SEQ ID NO: 38, with 0 to 2 nucleic acid substitutions. In some cases, an antisense oligomer provided herein comprises the sequence of SEQ ID NO: 38, with 0 to 1 nucleic acid substitution.

In some cases, an antisense oligomer provided herein comprises the sequence of SEQ ID NO: 39, with 0 to 4 nucleic acid substitutions. In some cases, an antisense oligomer provided herein comprises the sequence of SEQ ID NO: 39, with 0 to 3 nucleic acid substitutions. In some cases, an antisense oligomer provided herein comprises the sequence of SEQ ID NO: 39, with 0 to 2 nucleic acid substitutions. In some cases, an antisense oligomer provided herein comprises the sequence of SEQ ID NO: 39, with 0 to 1 nucleic acid substitution.

In some cases, an antisense oligomer provided herein comprises the sequence of SEQ ID NO: 40, with 0 to 4 nucleic acid substitutions. In some cases, an antisense oligomer provided herein comprises the sequence of SEQ ID NO: 40, with 0 to 3 nucleic acid substitutions. In some cases, an antisense oligomer provided herein comprises the sequence of SEQ ID NO: 40, with 0 to 2 nucleic acid substitutions. In some cases, an antisense oligomer provided herein comprises the sequence of SEQ ID NO: 40, with 0 to 1 nucleic acid substitution.

In some cases, an antisense oligomer provided herein comprises the sequence of SEQ ID NO: 41, with 0 to 4 nucleic acid substitutions. In some cases, an antisense oligomer provided herein comprises the sequence of SEQ ID NO: 41, with 0 to 3 nucleic acid substitutions. In some cases, an antisense oligomer provided herein comprises the sequence of SEQ ID NO: 41, with 0 to 2 nucleic acid substitutions. In some cases, an antisense oligomer provided herein comprises the sequence of SEQ ID NO: 41, with 0 to 1 nucleic acid substitution.

In some cases, an antisense oligomer provided herein comprises the sequence of SEQ ID NO: 42, with 0 to 4 nucleic acid substitutions. In some cases, an antisense oligomer provided herein comprises the sequence of SEQ ID NO: 42, with 0 to 3 nucleic acid substitutions. In some cases, an antisense oligomer provided herein comprises the sequence of SEQ ID NO: 42, with 0 to 2 nucleic acid substitutions. In some cases, an antisense oligomer provided herein comprises the sequence of SEQ ID NO: 42, with 0 to 1 nucleic acid substitution.

In some cases, an antisense oligomer provided herein comprises the sequence of SEQ ID NO: 43, with 0 to 4 nucleic acid substitutions. In some cases, an antisense oligomer provided herein comprises the sequence of SEQ ID NO: 43, with 0 to 3 nucleic acid substitutions. In some cases, an antisense oligomer provided herein comprises the sequence of SEQ ID NO: 43, with 0 to 2 nucleic acid substitutions. In some cases, an antisense oligomer provided herein comprises the sequence of SEQ ID NO: 43, with 0 to 1 nucleic acid substitution.

In some cases, an antisense oligomer provided herein comprises the sequence of SEQ ID NO: 44, with 0 to 4 nucleic acid substitutions. In some cases, an antisense oligomer provided herein comprises the sequence of SEQ ID NO: 44, with 0 to 3 nucleic acid substitutions. In some cases, an antisense oligomer provided herein comprises the sequence of SEQ ID NO: 44, with 0 to 2 nucleic acid substitutions. In some cases, an antisense oligomer provided herein comprises the sequence of SEQ ID NO: 44, with 0 to 1 nucleic acid substitution.

In some cases, an antisense oligomer provided herein comprises the sequence of SEQ ID NO: 45, with 0 to 4 nucleic acid substitutions. In some cases, an antisense oligomer provided herein comprises the sequence of SEQ ID NO: 45, with 0 to 3 nucleic acid substitutions. In some cases, an antisense oligomer provided herein comprises the sequence of SEQ ID NO: 45, with 0 to 2 nucleic acid substitutions. In some cases, an antisense oligomer provided herein comprises the sequence of SEQ ID NO: 45, with 0 to 1 nucleic acid substitution.

In some cases, an antisense oligomer provided herein comprises the sequence of SEQ ID NO: 46, with 0 to 4 nucleic acid substitutions. In some cases, an antisense oligomer provided herein comprises the sequence of SEQ ID NO: 46, with 0 to 3 nucleic acid substitutions. In some cases, an antisense oligomer provided herein comprises the sequence of SEQ ID NO: 46, with 0 to 2 nucleic acid substitutions. In some cases, an antisense oligomer provided herein comprises the sequence of SEQ ID NO: 46, with 0 to 1 nucleic acid substitution.

In some cases, an antisense oligomer provided herein comprises the sequence of SEQ ID NO: 47, with 0 to 4 nucleic acid substitutions. In some cases, an antisense oligomer provided herein comprises the sequence of SEQ ID NO: 47, with 0 to 3 nucleic acid substitutions. In some cases, an antisense oligomer provided herein comprises the sequence of SEQ ID NO: 47, with 0 to 2 nucleic acid substitutions. In some cases, an antisense oligomer provided herein comprises the sequence of SEQ ID NO: 47, with 0 to 1 nucleic acid substitution.

In some cases, an antisense oligomer provided herein comprises the sequence of SEQ ID NO: 48, with 0 to 4 nucleic acid substitutions. In some cases, an antisense oligomer provided herein comprises the sequence of SEQ ID NO: 48, with 0 to 3 nucleic acid substitutions. In some cases, an antisense oligomer provided herein comprises the sequence of SEQ ID NO: 48, with 0 to 2 nucleic acid substitutions. In some cases, an antisense oligomer provided herein comprises the sequence of SEQ ID NO: 48, with 0 to 1 nucleic acid substitution.

In some cases, an antisense oligomer provided herein comprises the sequence of SEQ ID NO: 49, with 0 to 4 nucleic acid substitutions. In some cases, an antisense oligomer provided herein comprises the sequence of SEQ ID NO: 49, with 0 to 3 nucleic acid substitutions. In some cases, an antisense oligomer provided herein comprises the sequence of SEQ ID NO: 49, with 0 to 2 nucleic acid substitutions. In some cases, an antisense oligomer provided herein comprises the sequence of SEQ ID NO: 49, with 0 to 1 nucleic acid substitution.

In some cases, an antisense oligomer provided herein comprises the sequence of SEQ ID NO: 50, with 0 to 4 nucleic acid substitutions. In some cases, an antisense oligomer provided herein comprises the sequence of SEQ ID NO: 50, with 0 to 3 nucleic acid substitutions. In some cases, an antisense oligomer provided herein comprises the sequence of SEQ ID NO: 50, with 0 to 2 nucleic acid substitutions. In some cases, an antisense oligomer provided herein comprises the sequence of SEQ ID NO: 50, with 0 to 1 nucleic acid substitution.

In some cases, an antisense oligomer provided herein comprises the sequence of SEQ ID NO: 51, with 0 to 4 nucleic acid substitutions. In some cases, an antisense oligomer provided herein comprises the sequence of SEQ ID NO: 51, with 0 to 3 nucleic acid substitutions. In some cases, an antisense oligomer provided herein comprises the sequence of SEQ ID NO: 51, with 0 to 2 nucleic acid substitutions. In some cases, an antisense oligomer provided herein comprises the sequence of SEQ ID NO: 51, with 0 to 1 nucleic acid substitution.

In some cases, an antisense oligomer provided herein comprises the sequence of SEQ ID NO: 52, with 0 to 4 nucleic acid substitutions. In some cases, an antisense oligomer provided herein comprises the sequence of SEQ ID NO: 52, with 0 to 3 nucleic acid substitutions. In some cases, an antisense oligomer provided herein comprises the sequence of SEQ ID NO: 52, with 0 to 2 nucleic acid substitutions. In some cases, an antisense oligomer provided herein comprises the sequence of SEQ ID NO: 52, with 0 to 1 nucleic acid substitution.

In some cases, an antisense oligomer provided herein comprises the sequence of SEQ ID NO: 53, with 0 to 4 nucleic acid substitutions. In some cases, an antisense oligomer provided herein comprises the sequence of SEQ ID NO: 53, with 0 to 3 nucleic acid substitutions. In some cases, an antisense oligomer provided herein comprises the sequence of SEQ ID NO: 53, with 0 to 2 nucleic acid substitutions. In some cases, an antisense oligomer provided herein comprises the sequence of SEQ ID NO: 53, with 0 to 1 nucleic acid substitution.

In some cases, an antisense oligomer provided herein comprises the sequence of SEQ ID NO: 54, with 0 to 4 nucleic acid substitutions. In some cases, an antisense oligomer provided herein comprises the sequence of SEQ ID NO: 54, with 0 to 3 nucleic acid substitutions. In some cases, an antisense oligomer provided herein comprises the sequence of SEQ ID NO: 54, with 0 to 2 nucleic acid substitutions. In some cases, an antisense oligomer provided herein comprises the sequence of SEQ ID NO: 54, with 0 to 1 nucleic acid substitution.

In some cases, an antisense oligomer provided herein comprises the sequence of SEQ ID NO: 55, with 0 to 4 nucleic acid substitutions. In some cases, an antisense oligomer provided herein comprises the sequence of SEQ ID NO: 55, with 0 to 3 nucleic acid substitutions. In some cases, an antisense oligomer provided herein comprises the sequence of SEQ ID NO: 55, with 0 to 2 nucleic acid substitutions. In some cases, an antisense oligomer provided herein comprises the sequence of SEQ ID NO: 55, with 0 to 1 nucleic acid substitution.

In some cases, an antisense oligomer provided herein comprises the sequence of SEQ ID NO: 56, with 0 to 4 nucleic acid substitutions. In some cases, an antisense oligomer provided herein comprises the sequence of SEQ ID NO: 56, with 0 to 3 nucleic acid substitutions. In some cases, an antisense oligomer provided herein comprises the sequence of SEQ ID NO: 56, with 0 to 2 nucleic acid substitutions. In some cases, an antisense oligomer provided herein comprises the sequence of SEQ ID NO: 56, with 0 to 1 nucleic acid substitution.

In some cases, an antisense oligomer provided herein comprises the sequence of SEQ ID NO: 57, with 0 to 4 nucleic acid substitutions. In some cases, an antisense oligomer provided herein comprises the sequence of SEQ ID NO: 57, with 0 to 3 nucleic acid substitutions. In some cases, an antisense oligomer provided herein comprises the sequence of SEQ ID NO: 57, with 0 to 2 nucleic acid substitutions. In some cases, an antisense oligomer provided herein comprises the sequence of SEQ ID NO: 57, with 0 to 1 nucleic acid substitution.

In some cases, an antisense oligomer provided herein comprises the sequence of SEQ ID NO: 58, with 0 to 4 nucleic acid substitutions. In some cases, an antisense oligomer provided herein comprises the sequence of SEQ ID NO: 58, with 0 to 3 nucleic acid substitutions. In some cases, an antisense oligomer provided herein comprises the sequence of SEQ ID NO: 58, with 0 to 2 nucleic acid substitutions. In some cases, an antisense oligomer provided herein comprises the sequence of SEQ ID NO: 58, with 0 to 1 nucleic acid substitution.

In some cases, an antisense oligomer provided herein comprises the sequence of SEQ ID NO: 59, with 0 to 4 nucleic acid substitutions. In some cases, an antisense oligomer provided herein comprises the sequence of SEQ ID NO: 59, with 0 to 3 nucleic acid substitutions. In some cases, an antisense oligomer provided herein comprises the sequence of SEQ ID NO: 59, with 0 to 2 nucleic acid substitutions. In some cases, an antisense oligomer provided herein comprises the sequence of SEQ ID NO: 59, with 0 to 1 nucleic acid substitution.

In some cases, an antisense oligomer provided herein comprises the sequence of SEQ ID NO: 60, with 0 to 4 nucleic acid substitutions. In some cases, an antisense oligomer provided herein comprises the sequence of SEQ ID NO: 60, with 0 to 3 nucleic acid substitutions. In some cases, an antisense oligomer provided herein comprises the sequence of SEQ ID NO: 60, with 0 to 2 nucleic acid substitutions. In some cases, an antisense oligomer provided herein comprises the sequence of SEQ ID NO: 60, with 0 to 1 nucleic acid substitution.

In some cases, an antisense oligomer provided herein comprises the sequence of SEQ ID NO: 61, with 0 to 4 nucleic acid substitutions. In some cases, an antisense oligomer provided herein comprises the sequence of SEQ ID NO: 61, with 0 to 3 nucleic acid substitutions. In some cases, an antisense oligomer provided herein comprises the sequence of SEQ ID NO: 61, with 0 to 2 nucleic acid substitutions. In some cases, an antisense oligomer provided herein comprises the sequence of SEQ ID NO: 61, with 0 to 1 nucleic acid substitution.

In some cases, an antisense oligomer provided herein comprises the sequence of SEQ ID NO: 62, with 0 to 4 nucleic acid substitutions. In some cases, an antisense oligomer provided herein comprises the sequence of SEQ ID NO: 62, with 0 to 3 nucleic acid substitutions. In some cases, an antisense oligomer provided herein comprises the sequence of SEQ ID NO: 62, with 0 to 2 nucleic acid substitutions. In some cases, an antisense oligomer provided herein comprises the sequence of SEQ ID NO: 62, with 0 to 1 nucleic acid substitution.

In some cases, an antisense oligomer provided herein comprises the sequence of SEQ ID NO: 63, with 0 to 4 nucleic acid substitutions. In some cases, an antisense oligomer provided herein comprises the sequence of SEQ ID NO: 63, with 0 to 3 nucleic acid substitutions. In some cases, an antisense oligomer provided herein comprises the sequence of SEQ ID NO: 63, with 0 to 2 nucleic acid substitutions. In some cases, an antisense oligomer provided herein comprises the sequence of SEQ ID NO: 63, with 0 to 1 nucleic acid substitution.

In some cases, an antisense oligomer provided herein comprises the sequence of SEQ ID NO: 64, with 0 to 4 nucleic acid substitutions. In some cases, an antisense oligomer provided herein comprises the sequence of SEQ ID NO: 64, with 0 to 3 nucleic acid substitutions. In some cases, an antisense oligomer provided herein comprises the sequence of SEQ ID NO: 64, with 0 to 2 nucleic acid substitutions. In some cases, an antisense oligomer provided herein comprises the sequence of SEQ ID NO: 64, with 0 to 1 nucleic acid substitution.

In some cases, an antisense oligomer provided herein comprises the sequence of SEQ ID NO: 65, with 0 to 4 nucleic acid substitutions. In some cases, an antisense oligomer provided herein comprises the sequence of SEQ ID NO: 65, with 0 to 3 nucleic acid substitutions. In some cases, an antisense oligomer provided herein comprises the sequence of SEQ ID NO: 65, with 0 to 2 nucleic acid substitutions. In some cases, an antisense oligomer provided herein comprises the sequence of SEQ ID NO: 65, with 0 to 1 nucleic acid substitution.

In some cases, an antisense oligomer provided herein comprises the sequence of SEQ ID NO: 66, with 0 to 4 nucleic acid substitutions. In some cases, an antisense oligomer provided herein comprises the sequence of SEQ ID NO: 66, with 0 to 3 nucleic acid substitutions. In some cases, an antisense oligomer provided herein comprises the sequence of SEQ ID NO: 66, with 0 to 2 nucleic acid substitutions. In some cases, an antisense oligomer provided herein comprises the sequence of SEQ ID NO: 66, with 0 to 1 nucleic acid substitution.

In some cases, an antisense oligomer provided herein comprises the sequence of SEQ ID NO: 67, with 0 to 4 nucleic acid substitutions. In some cases, an antisense oligomer provided herein comprises the sequence of SEQ ID NO: 67, with 0 to 3 nucleic acid substitutions. In some cases, an antisense oligomer provided herein comprises the sequence of SEQ ID NO: 67, with 0 to 2 nucleic acid substitutions. In some cases, an antisense oligomer provided herein comprises the sequence of SEQ ID NO: 67, with 0 to 1 nucleic acid substitution.

In some cases, an antisense oligomer provided herein comprises the sequence of SEQ ID NO: 68, with 0 to 4 nucleic acid substitutions. In some cases, an antisense oligomer provided herein comprises the sequence of SEQ ID NO: 68, with 0 to 3 nucleic acid substitutions. In some cases, an antisense oligomer provided herein comprises the sequence of SEQ ID NO: 68, with 0 to 2 nucleic acid substitutions. In some cases, an antisense oligomer provided herein comprises the sequence of SEQ ID NO: 68, with 0 to 1 nucleic acid substitution.

In some cases, an antisense oligomer provided herein comprises the sequence of SEQ ID NO: 69, with 0 to 4 nucleic acid substitutions. In some cases, an antisense oligomer provided herein comprises the sequence of SEQ ID NO: 69, with 0 to 3 nucleic acid substitutions. In some cases, an antisense oligomer provided herein comprises the sequence of SEQ ID NO: 69, with 0 to 2 nucleic acid substitutions. In some cases, an antisense oligomer provided herein comprises the sequence of SEQ ID NO: 69, with 0 to 1 nucleic acid substitution.

In some cases, an antisense oligomer provided herein comprises the sequence of SEQ ID NO: 70, with 0 to 4 nucleic acid substitutions. In some cases, an antisense oligomer provided herein comprises the sequence of SEQ ID NO: 70, with 0 to 3 nucleic acid substitutions. In some cases, an antisense oligomer provided herein comprises the sequence of SEQ ID NO: 70, with 0 to 2 nucleic acid substitutions. In some cases, an antisense oligomer provided herein comprises the sequence of SEQ ID NO: 70, with 0 to 1 nucleic acid substitution.

In some cases, an antisense oligomer provided herein comprises the sequence of SEQ ID NO: 71, with 0 to 4 nucleic acid substitutions. In some cases, an antisense oligomer provided herein comprises the sequence of SEQ ID NO: 71, with 0 to 3 nucleic acid substitutions. In some cases, an antisense oligomer provided herein comprises the sequence of SEQ ID NO: 71, with 0 to 2 nucleic acid substitutions. In some cases, an antisense oligomer provided herein comprises the sequence of SEQ ID NO: 71, with 0 to 1 nucleic acid substitution.

In some cases, an antisense oligomer provided herein comprises the sequence of SEQ ID NO: 72, with 0 to 4 nucleic acid substitutions. In some cases, an antisense oligomer provided herein comprises the sequence of SEQ ID NO: 72, with 0 to 3 nucleic acid substitutions. In some cases, an antisense oligomer provided herein comprises the sequence of SEQ ID NO: 72, with 0 to 2 nucleic acid substitutions. In some cases, an antisense oligomer provided herein comprises the sequence of SEQ ID NO: 72, with 0 to 1 nucleic acid substitution.

In some cases, an antisense oligomer provided herein comprises the sequence of SEQ ID NO: 73, with 0 to 4 nucleic acid substitutions. In some cases, an antisense oligomer provided herein comprises the sequence of SEQ ID NO: 73, with 0 to 3 nucleic acid substitutions. In some cases, an antisense oligomer provided herein comprises the sequence of SEQ ID NO: 73, with 0 to 2 nucleic acid substitutions. In some cases, an antisense oligomer provided herein comprises the sequence of SEQ ID NO: 73, with 0 to 1 nucleic acid substitution.

In some cases, an antisense oligomer provided herein comprises the sequence of SEQ ID NO: 74, with 0 to 4 nucleic acid substitutions. In some cases, an antisense oligomer provided herein comprises the sequence of SEQ ID NO: 74, with 0 to 3 nucleic acid substitutions. In some cases, an antisense oligomer provided herein comprises the sequence of SEQ ID NO: 74, with 0 to 2 nucleic acid substitutions. In some cases, an antisense oligomer provided herein comprises the sequence of SEQ ID NO: 74, with 0 to 1 nucleic acid substitution.

In some cases, an antisense oligomer provided herein comprises the sequence of SEQ ID NO: 75, with 0 to 4 nucleic acid substitutions. In some cases, an antisense oligomer provided herein comprises the sequence of SEQ ID NO: 75, with 0 to 3 nucleic acid substitutions. In some cases, an antisense oligomer provided herein comprises the sequence of SEQ ID NO: 75, with 0 to 2 nucleic acid substitutions. In some cases, an antisense oligomer provided herein comprises the sequence of SEQ ID NO: 75, with 0 to 1 nucleic acid substitution.

In some cases, an antisense oligomer provided herein comprises the sequence of SEQ ID NO: 76, with 0 to 4 nucleic acid substitutions. In some cases, an antisense oligomer provided herein comprises the sequence of SEQ ID NO: 76, with 0 to 3 nucleic acid substitutions. In some cases, an antisense oligomer provided herein comprises the sequence of SEQ ID NO: 76, with 0 to 2 nucleic acid substitutions. In some cases, an antisense oligomer provided herein comprises the sequence of SEQ ID NO: 76, with 0 to 1 nucleic acid substitution.

In some cases, an antisense oligomer provided herein comprises the sequence of SEQ ID NO: 77, with 0 to 4 nucleic acid substitutions. In some cases, an antisense oligomer provided herein comprises the sequence of SEQ ID NO: 77, with 0 to 3 nucleic acid substitutions. In some cases, an antisense oligomer provided herein comprises the sequence of SEQ ID NO: 77, with 0 to 2 nucleic acid substitutions. In some cases, an antisense oligomer provided herein comprises the sequence of SEQ ID NO: 77, with 0 to 1 nucleic acid substitution.

In some cases, an antisense oligomer provided herein comprises the sequence of SEQ ID NO: 78, with 0 to 4 nucleic acid substitutions. In some cases, an antisense oligomer provided herein comprises the sequence of SEQ ID NO: 78, with 0 to 3 nucleic acid substitutions. In some cases, an antisense oligomer provided herein comprises the sequence of SEQ ID NO: 78, with 0 to 2 nucleic acid substitutions. In some cases, an antisense oligomer provided herein comprises the sequence of SEQ ID NO: 78, with 0 to 1 nucleic acid substitution.

In some cases, an antisense oligomer provided herein comprises the sequence of SEQ ID NO: 79, with 0 to 4 nucleic acid substitutions. In some cases, an antisense oligomer provided herein comprises the sequence of SEQ ID NO: 79, with 0 to 3 nucleic acid substitutions. In some cases, an antisense oligomer provided herein comprises the sequence of SEQ ID NO: 79, with 0 to 2 nucleic acid substitutions. In some cases, an antisense oligomer provided herein comprises the sequence of SEQ ID NO: 79, with 0 to 1 nucleic acid substitution.

In some cases, an antisense oligomer provided herein comprises the sequence of SEQ ID NO: 80, with 0 to 4 nucleic acid substitutions. In some cases, an antisense oligomer provided herein comprises the sequence of SEQ ID NO: 80, with 0 to 3 nucleic acid substitutions. In some cases, an antisense oligomer provided herein comprises the sequence of SEQ ID NO: 80, with 0 to 2 nucleic acid substitutions. In some cases, an antisense oligomer provided herein comprises the sequence of SEQ ID NO: 80, with 0 to 1 nucleic acid substitution.

In some cases, an antisense oligomer provided herein comprises the sequence of SEQ ID NO: 81, with 0 to 4 nucleic acid substitutions. In some cases, an antisense oligomer provided herein comprises the sequence of SEQ ID NO: 81, with 0 to 3 nucleic acid substitutions. In some cases, an antisense oligomer provided herein comprises the sequence of SEQ ID NO: 81, with 0 to 2 nucleic acid substitutions. In some cases, an antisense oligomer provided herein comprises the sequence of SEQ ID NO: 81, with 0 to 1 nucleic acid substitution.

In some cases, an antisense oligomer provided herein comprises the sequence of SEQ ID NO: 82, with 0 to 4 nucleic acid substitutions. In some cases, an antisense oligomer provided herein comprises the sequence of SEQ ID NO: 82, with 0 to 3 nucleic acid substitutions. In some cases, an antisense oligomer provided herein comprises the sequence of SEQ ID NO: 82, with 0 to 2 nucleic acid substitutions. In some cases, an antisense oligomer provided herein comprises the sequence of SEQ ID NO: 82, with 0 to 1 nucleic acid substitution.

In some cases, an antisense oligomer provided herein comprises the sequence of SEQ ID NO: 83, with 0 to 4 nucleic acid substitutions. In some cases, an antisense oligomer provided herein comprises the sequence of SEQ ID NO: 83, with 0 to 3 nucleic acid substitutions. In some cases, an antisense oligomer provided herein comprises the sequence of SEQ ID NO: 83, with 0 to 2 nucleic acid substitutions. In some cases, an antisense oligomer provided herein comprises the sequence of SEQ ID NO: 83, with 0 to 1 nucleic acid substitution.

In some cases, an antisense oligomer provided herein comprises the sequence of SEQ ID NO: 84, with 0 to 4 nucleic acid substitutions. In some cases, an antisense oligomer provided herein comprises the sequence of SEQ ID NO: 84, with 0 to 3 nucleic acid substitutions. In some cases, an antisense oligomer provided herein comprises the sequence of SEQ ID NO: 84, with 0 to 2 nucleic acid substitutions. In some cases, an antisense oligomer provided herein comprises the sequence of SEQ ID NO: 84, with 0 to 1 nucleic acid substitution.

In some cases, an antisense oligomer provided herein comprises the sequence of SEQ ID NO: 85, with 0 to 4 nucleic acid substitutions. In some cases, an antisense oligomer provided herein comprises the sequence of SEQ ID NO: 85, with 0 to 3 nucleic acid substitutions. In some cases, an antisense oligomer provided herein comprises the sequence of SEQ ID NO: 85, with 0 to 2 nucleic acid substitutions. In some cases, an antisense oligomer provided herein comprises the sequence of SEQ ID NO: 85, with 0 to 1 nucleic acid substitution.

In some cases, an antisense oligomer provided herein comprises the sequence of SEQ ID NO: 86, with 0 to 4 nucleic acid substitutions. In some cases, an antisense oligomer provided herein comprises the sequence of SEQ ID NO: 86, with 0 to 3 nucleic acid substitutions. In some cases, an antisense oligomer provided herein comprises the sequence of SEQ ID NO: 86, with 0 to 2 nucleic acid substitutions. In some cases, an antisense oligomer provided herein comprises the sequence of SEQ ID NO: 86, with 0 to 1 nucleic acid substitution.

In some cases, an antisense oligomer provided herein comprises the sequence of SEQ ID NO: 87, with 0 to 4 nucleic acid substitutions. In some cases, an antisense oligomer provided herein comprises the sequence of SEQ ID NO: 87, with 0 to 3 nucleic acid substitutions. In some cases, an antisense oligomer provided herein comprises the sequence of SEQ ID NO: 87, with 0 to 2 nucleic acid substitutions. In some cases, an antisense oligomer provided herein comprises the sequence of SEQ ID NO: 87, with 0 to 1 nucleic acid substitution.

In some cases, an antisense oligomer provided herein comprises the sequence of SEQ ID NO: 88, with 0 to 4 nucleic acid substitutions. In some cases, an antisense oligomer provided herein comprises the sequence of SEQ ID NO: 88, with 0 to 3 nucleic acid substitutions. In some cases, an antisense oligomer provided herein comprises the sequence of SEQ ID NO: 88, with 0 to 2 nucleic acid substitutions. In some cases, an antisense oligomer provided herein comprises the sequence of SEQ ID NO: 88, with 0 to 1 nucleic acid substitution.

In some cases, an antisense oligomer provided herein comprises the sequence of SEQ ID NO: 89, with 0 to 4 nucleic acid substitutions. In some cases, an antisense oligomer provided herein comprises the sequence of SEQ ID NO: 89, with 0 to 3 nucleic acid substitutions. In some cases, an antisense oligomer provided herein comprises the sequence of SEQ ID NO: 89, with 0 to 2 nucleic acid substitutions. In some cases, an antisense oligomer provided herein comprises the sequence of SEQ ID NO: 89, with 0 to 1 nucleic acid substitution.

In some cases, an antisense oligomer provided herein comprises the sequence of SEQ ID NO: 90, with 0 to 4 nucleic acid substitutions. In some cases, an antisense oligomer provided herein comprises the sequence of SEQ ID NO: 90, with 0 to 3 nucleic acid substitutions. In some cases, an antisense oligomer provided herein comprises the sequence of SEQ ID NO: 90, with 0 to 2 nucleic acid substitutions. In some cases, an antisense oligomer provided herein comprises the sequence of SEQ ID NO: 90, with 0 to 1 nucleic acid substitution.

In some cases, an antisense oligomer provided herein comprises the sequence of SEQ ID NO: 91, with 0 to 4 nucleic acid substitutions. In some cases, an antisense oligomer provided herein comprises the sequence of SEQ ID NO: 91, with 0 to 3 nucleic acid substitutions. In some cases, an antisense oligomer provided herein comprises the sequence of SEQ ID NO: 91, with 0 to 2 nucleic acid substitutions. In some cases, an antisense oligomer provided herein comprises the sequence of SEQ ID NO: 91, with 0 to 1 nucleic acid substitution.

In some cases, an antisense oligomer provided herein comprises the sequence of SEQ ID NO: 92, with 0 to 4 nucleic acid substitutions. In some cases, an antisense oligomer provided herein comprises the sequence of SEQ ID NO: 92, with 0 to 3 nucleic acid substitutions. In some cases, an antisense oligomer provided herein comprises the sequence of SEQ ID NO: 92, with 0 to 2 nucleic acid substitutions. In some cases, an antisense oligomer provided herein comprises the sequence of SEQ ID NO: 92, with 0 to 1 nucleic acid substitution.

In certain embodiments, an antisense oligomer targeted to a target nucleic acid is 12 to 30 subunits in length. In certain embodiments, an antisense oligomer targeted to a target nucleic acid is 12 to 25 subunits in length. In certain embodiments, an antisense oligomer targeted to a target nucleic acid is 12 to 22 subunits in length. In certain embodiments, an antisense oligomer targeted to a target nucleic acid is 14 to 20 subunits in length. In certain embodiments, an antisense oligomer targeted to a target nucleic acid is 15 to 25 subunits in length. In certain embodiments, an antisense oligomer targeted to a target nucleic acid is 18 to 22 subunits in length. In certain embodiments, an antisense oligomer targeted to a target nucleic acid is 19 to 21 subunits in length. In certain embodiments, the antisense oligomer is 8 to 80, 12 to 50, 13 to 30, 13 to 50, 14 to 30, 14 to 50, 15 to 30, 15 to 50, 16 to 30, 16 to 50, 17 to 30, 17 to 50, 18 to 30, 18 to 50, 19 to 30, 19 to 50, or 20 to 30 linked subunits in length.

In certain embodiments, an antisense oligomer targeted to a target nucleic acid is 12 subunits in length. In certain embodiments, an antisense oligomer targeted to a target nucleic acid is 13 subunits in length. In certain embodiments, an antisense oligomer targeted to a target nucleic acid is 14 subunits in length. In certain embodiments, an antisense oligomer targeted to a target nucleic acid is 15 subunits in length. In certain embodiments, an antisense oligomer targeted to a target nucleic acid is 16 subunits in length. In certain embodiments, an antisense oligomer targeted to a target nucleic acid is 17 subunits in length. In certain embodiments, an antisense oligomer targeted to a target nucleic acid is 18 subunits in length. In certain embodiments, an antisense oligomer targeted to a target nucleic acid is 19 subunits in length. In certain embodiments, an antisense oligomer targeted to a target nucleic acid is 20 subunits in length. In certain embodiments, an antisense oligomer targeted to a target nucleic acid is 21 subunits in length. In certain embodiments, an antisense oligomer targeted to a target nucleic acid is 22 subunits in length. In certain embodiments, an antisense oligomer targeted to a target nucleic acid is 23 subunits in length. In certain embodiments, an antisense oligomer targeted to a target nucleic acid is 24 subunits in length. In certain embodiments, an antisense oligomer targeted to a target nucleic acid is 25 subunits in length. In certain embodiments, an antisense oligomer targeted to a target nucleic acid is 26 subunits in length. In certain embodiments, an antisense oligomer targeted to a target nucleic acid is 27 subunits in length. In certain embodiments, an antisense oligomer targeted to a target nucleic acid is 28 subunits in length. In certain embodiments, an antisense oligomer targeted to a target nucleic acid is 29 subunits in length. In certain embodiments, an antisense oligomer targeted to a target nucleic acid is 30 subunits in length. In certain embodiments, the antisense oligomer targeted to a target nucleic acid is 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. In certain embodiments the antisense oligomer is an antisense oligonucleotide, and the linked subunits are nucleosides.

Antisense oligomers provided herein can have nucleotides that mismatch the target sequence. For instance, an antisense oligonucleotide of 25 nucleobases in length can have 8 or 11 mismatch bases near the ends of the antisense oligonucleotides, while still being able to direct specific cleavage of the target mRNA, albeit to a lesser extent than the antisense oligonucleotides that contained no mismatches. In some cases, the antisense oligonucleotide provided herein has 12 to 30 subunits in length (e.g., nucleobases), including those with 1 or 3 mismatches.

### Chemically Modified Antisense Oligomer

In certain embodiments, antisense oligomers provided herein have chemically modified subunits arranged in patterns, or motifs, to confer to the antisense oligomers properties such as enhanced inhibitory activity, increased binding affinity for a target nucleic acid, or resistance to degradation by in vivo nucleases.

In some cases, provided herein are chimeric antisense oligomers. For instance, chimeric antisense oligomers can contain at least one region modified so as to confer increased resistance to nuclease degradation, increased cellular uptake, increased binding affinity for the target nucleic acid, and/or increased inhibitory activity. A second region of a chimeric antisense oligomer can optionally serve as a substrate for the cellular endonuclease RNase H, which cleaves the RNA strand of an RNA:DNA duplex.

In some cases, the antisense oligomers provided herein have a gapmer motif. Antisense oligomers having a gapmer motif can be considered chimeric antisense oligomers. In a gapmer, an internal region having a plurality of nucleotides that supports RNaseH cleavage can be positioned between external regions having a plurality of nucleotides that are chemically distinct from the nucleosides of the internal region. In the case of an antisense oligonucleotide having a gapmer motif, the gap segment can serve as the substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer can include β-D-ribonucleosides, β-D-deoxyribonucleosides, 2′-modified nucleosides (such 2′-modified nucleosides may include 2′-MOE, and 2′-O—CH₃, among others), and bicyclic sugar modified nucleosides (such bicyclic sugar modified nucleosides may include those having a 4′-(CH₂)n-O-2′ bridge, where n=1 or n=2 and 4′-CH₂—O—CH₂-2′). In certain embodiments, wings include several modified sugar moieties, including, for example, 2′-MOE. In certain embodiments, wings include several modified and unmodified sugar moieties. In certain embodiments, wings include various combinations of 2′-MOE nucleosides and 2′-deoxynucleosides.

Each distinct region can comprise uniform sugar moieties, variant, or alternating sugar moieties. The wing-gap-wing motif is frequently described as “X-Y-Z”, where “X” represents the length of the 5′ wing, “Y” represents the length of the gap, and “Z” represents the length of the 3′ wing. “X” and “Z” can comprise uniform, variant, or alternating sugar moieties. In certain embodiments, “X” and “Y” include one or more 2′-deoxynucleosides. “Y” can comprise 2′-deoxynucleosides. As used herein, a gapmer described as “X-Y-Z” can have a configuration such that the gap is positioned immediately adjacent to each of the 5′ wing and the 3′ wing. Thus, no intervening nucleotides can exist between the 5′ wing and gap, or the gap and the 3′ wing. Any of the antisense oligomers described herein can have a gapmer motif. In certain embodiments, “X” and “Z” are the same; in other cases they are different.

In certain cases, gapmers provided herein include, for example, 20-mers having a motif of 5-10-5 in the form of “X-Y-Z” as described herein. In certain embodiments, gapmers provided herein include, for example, 19-mers having a motif of 5-9-5 in the form of “X-Y-Z” as described herein. In certain embodiments, gapmers provided herein include, for example, 18-mers having a motif of 5-8-5 in the form of “X-Y-Z” as described herein. In certain embodiments, gapmers provided herein include, for example, 18-mers having a motif of 4-8-6 in the form of “X-Y-Z” as described herein. In certain embodiments, gapmers provided herein include, for example, 18-mers having a motif of 6-8-4 in the form of “X-Y-Z” as described herein. In certain embodiments, gapmers provided herein include, for example, 18-mers having a motif of 5-7-6 in the form of “X-Y-Z” as described herein.

In some cases, the antisense oligomer comprises: a 5′ region consisting of three, four, five, or six linked nucleosides (e.g., “X” part discussed above); a central region consisting of eight, nine, ten, eleven, or twelve linked nucleosides(e.g., “Y” part discussed above); and a 3′ region consisting of three, four, five, or six linked nucleosides (e.g., “Z” part discussed above). In some cases, each of the three, four, five, or six linked nucleosides in the 5′ region and each of three, four, five, or six linked nucleosides in the 3′ region comprise a modified sugar moiety, and each of the eight, nine, ten, eleven, or twelve linked nucleosides in the central region is a deoxyribonucleoside. In some cases, the modified sugar moiety includes a 2′-O-methyl moiety, a 2′-Fluoro moiety, a 2′-O-methoxyethyl moiety, or a 2′-NMA moiety, or any combination thereof. In some cases, one or more the nucleosides in the 5′ region and in the 3′ region further comprise other modification as disclosed herein. In some cases, all the nucleosides in the 5′ region and in the 3′ region further comprise other modification as disclosed herein.

### Complementarity

An agent provided herein can have a polynucleotide sequence complementary to a target nucleic acid when a sufficient number of nucleobases in the polynucleotide sequence (for instance antisense oligomer) can hydrogen bond with the corresponding nucleobases of the target nucleic acid, such that a desired effect can occur (e.g., antisense inhibition of a target nucleic acid, such as a UBE3A nucleic acid).

Non-complementary nucleobases between an agent (e.g., an antisense oligomer) and a target nucleic acid may be tolerated provided that the agent (e.g., antisense oligomer) remains able to specifically hybridize to a target nucleic acid. Moreover, an agent (e.g., antisense oligomer) can hybridize to one or more segments of a target nucleic acid such that intervening or adjacent segments are not involved in the hybridization event (e.g., a loop structure, mismatch or hairpin structure).

In certain embodiments, the agents (e.g., antisense oligomers) provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a UBE3A nucleic acid, a target region, target segment, or specified portion thereof. Percent complementarity of an antisense oligomer with a target nucleic acid can be determined using routine methods, such as using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, the agents (e.g., antisense oligomers) provided herein, or specified portions thereof, are fully complementary (i.e., 100% complementary) to a target nucleic acid, or specified portion thereof. For example, agents (e.g., antisense oligomers) provided herein can be fully complementary to a UBE3A nucleic acid, or a target region, or a target segment or target sequence thereof. As used herein, “fully complementary” can mean that each nucleobase of an antisense oligomer is capable of precise base pairing with the corresponding nucleobases of a target nucleic acid.

The location of a non-complementary nucleobase can be at the 5′ end or 3′ end of the antisense oligomer. Alternatively, the non-complementary nucleobase or nucleobases can be at an internal position of the antisense oligomer. When two or more non-complementary nucleobases are present, they can be contiguous (i.e., linked) or non-contiguous. In one embodiment, a non-complementary nucleobase is located in the wing segment of a gapmer antisense oligonucleotide.

In certain embodiments, antisense oligomers provided herein that are, or are up to 1 1, 12, 13, 14, 15, 16, 17, 18, 19, or 20 nucleobases in length comprise no more than 4, no more than 3, no more than 2, or no more than 1 non-complementary nucleobase(s) relative to a target nucleic acid, or specified portion thereof.

In certain embodiments, antisense oligomers provided herein that are, or are up to 1 1, 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, or specified portion thereof.

The agents (e.g., antisense oligomers) provided herein can also include those which are complementary to a portion of a target nucleic acid. As used herein, “portion” can refer to a defined number of contiguous (i.e., linked) nucleobases within a region or segment of a target nucleic acid. A “portion” can also refer to a defined number of contiguous nucleobases of an antisense oligomer. In certain embodiments, the agents (e.g., antisense oligomers) are complementary to at least an 8 nucleobase portion of a target segment. In certain embodiments, the agents (e.g., antisense oligomers) are complementary to at least a 9 nucleobase portion of a target segment. In certain embodiments, the agents (e.g., antisense oligomers) are complementary to at least a 10 nucleobase portion of a target segment. In certain embodiments, the agents (e.g., antisense oligomers) are complementary to at least an 11 nucleobase portion of a target segment. In certain embodiments, the agents (e.g., antisense oligomers) are complementary to at least a 12 nucleobase portion of a target segment. In certain embodiments, the agents (e.g., antisense oligomers) are complementary to at least a 13 nucleobase portion of a target segment. In certain embodiments, the agents (e.g., antisense oligomers) are complementary to at least a 14 nucleobase portion of a target segment. In certain embodiments, the agents (e.g., antisense oligomers) are complementary to at least a 15 nucleobase portion of a target segment. Also contemplated are antisense oligomers that are complementary to at least a 9, 10, 1 1, 12, 13, 14, 15, 16, 17, 18, 19, 20, or more nucleobase portion of a target segment, or a range defined by any two of these values.

The agents (e.g., antisense oligomers) provided herein can also have a defined percent identity to a particular nucleotide sequence, SEQ ID NO, or portion thereof. As used herein, an antisense oligomer is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the antisense oligomers described herein as well as oligomers having non-identical bases relative to the antisense oligomers provided herein also are contemplated. The non-identical bases can be adjacent to each other or dispersed throughout the antisense oligomer. Percent identity of an antisense oligomer is calculated according to the number of bases that have identical base pairing relative to the sequence to which it is being compared.

In certain embodiments, the agents (e.g., antisense oligomers), or portions thereof, are at least 70%, 75%, 80%, 85%, 90%, 95%, 96%, 97%, 98%, 99% or 100% identical to one or more of the agents (e.g., antisense oligomers) or SEQ ID NOs, or a portion thereof, disclosed herein.

In certain embodiments, a portion of the agent (e.g., antisense oligomer) is compared to an equal length portion of the target nucleic acid. In certain embodiments, an 8, 9, 10, 1 1, 12, 13, 14, 15, 16, 17, 18, 19, 20, 21, 22, 23, 24, or 25 nucleobase portion is compared to an equal length portion of the target nucleic acid.

In certain embodiments, a portion of the antisense oligonucleotide is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

### Modifications

In some embodiments, an antisense oligomer provided herein can have one or more chemical modifications as compared to a naturally occurring nucleotide (or a native form of the antisense oligomer) that has the same or comparable polynucleotide sequence. Modifications to antisense oligomers encompass substitutions or changes to internucleoside linkages, sugar moieties, or nucleobases. Modified antisense oligomers can be preferred over native forms because of desirable properties such as, for example, enhanced cellular uptake, enhanced affinity for nucleic acid target, increased stability in the presence of nucleases, or increased inhibitory activity.

Chemically modified nucleosides can be employed to increase the binding affinity of a shortened or truncated antisense oligonucleotide for its target nucleic acid. Consequently, comparable results can often be obtained with shorter antisense oligomers that have such chemically modified nucleosides.

A nucleoside can be a base-sugar combination. The nucleobase (also known as base) portion of the nucleoside can be a heterocyclic base moiety in native form. Nucleotides are nucleosides that further include a phosphate group covalently linked to the sugar portion of the nucleoside. For those nucleosides that include a pentofuranosyl sugar, the phosphate group can be linked to the 2′, 3′, or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

### Modified Internucleoside Linkages

The naturally occurring internucleoside linkage of RNA and DNA is a 3′ to 5′ phosphodiester linkage. Antisense oligomers provided herein can have one or more modified, i.e., non-naturally occurring, internucleoside linkages. Antisense oligomers having one or more modified intemucleotide linkages can have desirable properties such as, for example, enhanced cellular uptake, enhanced affinity for target nucleic acids, and increased stability in the presence of nucleases.

Oligonucleotides having modified internucleoside linkages can include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates.

In certain embodiments, antisense oligomers targeted to a UBE3A nucleic acid comprise one or more modified internucleoside linkages. In certain embodiments, the modified intemucleoside linkages are interspersed throughout the antisense oligomer. In certain embodiments, the modified internucleoside linkages are phosphorothioate linkages. In certain embodiments, each internucleoside linkage of an antisense oligomer is a phosphorothioate internucleoside linkage.

### Modified Sugar Moieties

Antisense oligomers provided herein can contain one or more nucleosides wherein the sugar group has been modified. Such sugar modified nucleosides can impart enhanced nuclease stability, increased binding affinity, or some other beneficial biological property to the antisense oligomers. In certain embodiments, nucleosides comprise chemically modified ribofuranose ring moieties.

Examples of chemically modified ribofuranose rings include without limitation, addition of substitute groups (including 5′ and 2′ substituent groups, bridging of non-geminal ring atoms to form bicyclic nucleic acids (BNA), replacement of the ribosyl ring oxygen atom with S, N(R), or C(R₁)(R₂)(R, R₁ and R₂ are each independently H, C₁-C₁₂ alkyl or a protecting group) and combinations thereof. Examples of chemically modified sugars include 2′-F-5′-methyl substituted nucleoside (see PCT International Application WO 2008/101157 for other disclosed 5′,2′-bis substituted nucleosides) or replacement of the ribosyl ring oxygen atom with S with further substitution at the 2′-position (see published U.S. Patent Application US2005-0130923, published on Jun. 16, 2005) or alternatively 5′-substitution of a BNA (see PCT International Application WO 2007/134181 wherein LNA is substituted with, for example, a 5′-methyl or a 5′-vinyl group).

Examples of nucleosides having modified sugar moieties include without limitation nucleosides comprising 5′-vinyl, 5′-methyl (R or S), 4′-S, 2′-F, 2′-OCH₃, 2′-OCH₂CH₃, 2′-OCH₂CH₂F, 2′-NMA, and 2′-O(CH₂)₂OCH₃ substituent groups. The substituent at the 2′ position can also be selected from allyl, amino, azido, thio, 0-allyl, O—C₁-C₁₀ alkyl, OCF₃, OCH₂F, O(CH₂)₂SCH₃, O(CH₂)₂—O—N(Rₘ)(Rₙ), O—CH₂—C(═O)—N(Rₘ)(Rₙ), and O—CH₂—C(═O)—N(R)—(CH₂)₂—N(Rₘ)(Rₙ), where each R₁, Rₘ and Rₙ is, independently, H or substituted or unsubstituted C₁-C₁₀ alkyl.

As used herein, “bicyclic nucleosides” can refer to modified nucleosides comprising a bicyclic sugar moiety. Examples of bicyclic nucleosides include, without limitation, nucleosides comprising a bridge between the 4′ and the 2′ ribosyl ring atoms. In certain embodiments, antisense oligomers provided herein include one or more bicyclic nucleosides comprising a 4′ to 2′ bridge. Examples of such 4′ to 2′ bridged bicyclic nucleosides, include, but are not limited to, one of those described in U.S. Pat. Nos. 7,399,845, 8,278,283, U.S. Patent Application Pat. Nos. 7,696,345, 7,427,672, 8,278,426, 6,268,490; 6,525, 191; 6,670,461; 6,770,748; 6,794,499; 7,034, 133; 7,053,207, 7,399,845, 7,547,684, 7,741,457, and 7,696,345; U.S. Pat. Nos.; U.S. Patent Publication No. US2008-0039618; and Chattopadhyaya et al, J. Org. Chem., 2009, 74, 1 18-134); Singh et al., Chem. Commun., 1998, 4, 455-456; Koshkin et al., Tetrahedron, 1998, 54, 3607-3630; Wahlestedt et al, Proc. Natl. Acad. Sci. U.S.A, 2000, 97, 5633-5638; Kumar et al., Bioorg. Med. Chem. Lett., 1998, 8, 2219-2222; Singh et al, J. Org. Chem., 1998, 63, 10035-10039; Srivastava et al, J. Am. Chem. Soc, 2007, 129(26) 8362-8379; Elayadi et al, Curr. Opinion Invest. Drugs, 2001, 2, 558-561; Braasch et al, Chem. Biol, 2001, 8, 1-7; and Orum et al, Curr. Opinion Mol. Ther., 2001, 3, 239-243. Each of the foregoing bicyclic nucleosides can be prepared having one or more stereochemical sugar configurations including, for example, a-L-ribofuranose and β-D-ribofuranose (see PCT international application PCT/DK98/00393, published on Mar. 25, 1999 as WO 99/14226). In certain embodiments, bicyclic sugar moieties of BNA nucleosides include, but are not limited to, described in U.S. Pat. No. 11,129,844.

The synthesis and preparation of the methyleneoxy (4′-CH₂—O-2′) BNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630).

As used herein, “4′-2′ bicyclic nucleoside” or “4′ to 2′ bicyclic nucleoside” can refer to a bicyclic nucleoside comprising a furanose ring comprising a bridge connecting two carbon atoms of the furanose ring connects the 2′ carbon atom and the 4′ carbon atom of the sugar ring.

As used herein, “monocyclic nucleosides” can refer to nucleosides comprising modified sugar moieties that are not bicyclic sugar moieties. In certain embodiments, the sugar moiety, or sugar moiety analogue, of a nucleoside is modified or substituted at any position.

As used herein, “2′-modified sugar” can mean a furanosyl sugar modified at the 2′ position. In certain embodiments, such modifications include substituents selected from: a halide, including, but not limited to substituted and unsubstituted alkoxy, substituted and unsubstituted thioalkyl, substituted and unsubstituted amino alkyl, substituted and unsubstituted alkyl, substituted and unsubstituted allyl, and substituted and unsubstituted alkynyl. In certain embodiments, 2′ modifications are selected from substituents including, but not limited to: O[(CH₂)ₙO]ₘCH₃, O(CH₂)ₙNH₂, O(CH₂)ₙCH₃, O(CH₂)ₙF, O(CH₂)ₙONH₂, OCH₂C(═O)N(H)CH₃ and O(CH₂)ₙON[(CH₂)ₙCH₃]2, where n and m are from 1 to about 10. Other 2′-substituent groups can also be selected from: C₁-C₁₂ alkyl, substituted alkyl, alkenyl, alkynyl, alkaryl, aralkyl, O-alkaryl or O-aralkyl, SH, SCH₃, OCN, CI, Br, CN, F, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving pharmacokinetic properties, or a group for improving the pharmacodynamic properties of an antisense oligomer, and other substituents having similar properties. In certain embodiments, modified nucleosides comprise a 2′-MOE side chain (Baker et al., J. Biol. Chem., 1997, 272, 11944-12000). Such 2′-MOE substitution have been described as having improved binding affinity compared to unmodified nucleosides and to other modified nucleosides, such as 2′-O-methyl, O-propyl, and O-aminopropyl. Oligonucleotides having the 2′-MOE substituent also have been shown to be antisense inhibitors of gene expression with promising features for in vivo use (Martin, Helv. Chim. Acta, 1995, 78, 486-504; Altmann et al., Chimia, 1996, 50, 168-176; Altmann et al., Biochem. Soc. Trans., 1996, 24, 630-637; and Altmann et al, Nucleosides Nucleotides, 1997, 16, 917-926).

As used herein, “2′-NMA” can mean a —O—CH₂—C(═O)—NH—CH₃ group in place of the 2′-OH group of a ribosyl sugar moiety. A “2′-NMA sugar moiety” or “2′-NMA moiety” is a sugar moiety with a 2′-O—CH₂—C(═O)—NH—CH₃ group in place of the 2′—OH group of a nbosyl sugar moiety. Unless otherwise indicated, a 2′-NMA sugar moiety is in the β-D configuration. “NMA” can mean O—N-methyl acetamide.

As used herein, “2′-NMA nucleoside” can mean a nucleoside comprising a 2′-NMA sugar moiety.

As used herein, “2′-F” can refer to a nucleoside comprising a sugar comprising a fluoro group at the 2′ position.

As used herein, “2′-OMe” or “2′-OCH₃” or “2′-O-methyl” each can refer to a nucleoside comprising a sugar comprising an —OCH₃ group at the 2′ position of the sugar ring.

As used herein, “MOE” or “2′-MOE” or “2′-OCH₂CH₂OCH₃” or “2′-O-methoxyethyl” each refers to a nucleoside comprising a sugar comprising a —OCH₂CH₂OCH₃ group at the 2′ position of the sugar ring.

In certain embodiments, one or more of the plurality of nucleosides is modified. In certain embodiments, an oligonucleotide comprises one or more ribonucleosides (RNA) and/or deoxyribonucleosides (DNA). In certain embodiments, an oligonucleotide comprises a mix of one or more ribonucleosides (RNA) and deoxyribonucleosides (DNA).

Many other bicyclo and tricyclo sugar surrogate ring systems are also known in the art that can be used to modify nucleosides for incorporation into antisense oligomers (see, for example, review article: Leumann, Bioorg. Med. Chem., 2002, 10, 841-854). Such ring systems can undergo various additional substitutions to enhance activity.

Methods for the preparations of modified sugars are well known to those skilled in the art.

In nucleotides having modified sugar moieties, the nucleobase moieties (natural, modified or a combination thereof) are maintained for hybridization with an appropriate nucleic acid target.

In certain embodiments, antisense oligomers comprise one or more nucleosides having modified sugar moieties. In certain embodiments, the modified sugar moiety is 2′-MOE. In certain embodiments, the 2′-MOE modified nucleosides are arranged in a gapmer motif. In certain embodiments, the modified sugar moiety is a bicyclic nucleoside having a (4′-CH(CH₃)-0-2′) bridging group. In certain embodiments, the (4′-CH(CH₃)—O-2′) modified nucleosides are arranged throughout the wings of a gapmer motif.

“5′-methylcytosine” can mean a cytosine modified with a methyl group attached to the 5′ position. A 5′-methylcytosine is a modified nucleobase.

“5′-methyluracil” can mean a uracil modified with a methyl group attached to the 5′ position. A 5′-methyluracil is a modified nucleobase.

“5′-methylthymine” can mean a thymine modified with a methyl group attached to the 5′ position. A 5′-methylthymine is a modified nucleobase.

In some cases, antisense oligomers provided herein comprise 5′-methylcytosine, 5′-methyluracil, 5′-methylthymine, or a combination thereof. In some cases, each cytosine in the antisense oligomer is methylated, i.e., having a methyl group attached to the 5′ position. In some cases, each uracil in the antisense oligomer is methylated, i.e., having a methyl group attached to the 5′ position. In some cases, the antisense oligomer has 1, 2, 3, 4, 5, 6, 7, 8, or more 5′-methylcytosine. In some cases, the antisense oligomer has 1, 2, 3, 4, 5, 6, 7, 8, or more 5′-methyluracil. In some cases, the antisense oligomer has both methylcytosine and methyluracil.

### Pharmaceutical Compositions and Methods of Treatment

In some aspects, provided herein are pharmaceutical compositions comprising an agent of the present disclosure, e.g., an antisense oligomer, or a vector encoding the agent.

Pharmaceutical compositions or formulations comprising the agent, e.g., antisense oligomer, or a vector encoding the agent, of the described compositions and for use in any of the described methods can be prepared according to conventional techniques well known in the pharmaceutical industry and described in the published literature. In some embodiments, a pharmaceutical composition or formulation for treating a subject comprises an effective amount of any antisense oligomer as described herein, or a pharmaceutically acceptable salt, solvate, hydrate or ester thereof. The pharmaceutical formulation comprising an antisense oligomer may further comprise a pharmaceutically acceptable excipient, diluent or carrier.

In some cases, antisense oligomers provided herein have an intermediate level of efficiency in reducing the level of UBE3A transcript in cells, e.g., the antisense oligomer, when provided at a maximum dosing level (maximum concentration when delivered in vitro, maximum dose when in vivo, without causing significant adverse effects to the cells or the subject), can reduce UBE3A transcript level by at most 75%, at most 70%, at most 65%, at most 60%, at most 55%, at most 50%, at most 45%, at most 40%, at most 35%, at most 30%, at most 20%, or about 25% to about 75%, about 30% to about 70%, about 35% to about 65%, about 40% to about 60%, or about 45% to about 55%. Without wishing to be bound by a certain theory, antisense oligomers provided herein that have an intermediate level of UBE3A knockdown efficiency can have desirable therapeutic applications. Intermediate level of knockdown efficiency of an antisense oligomer can be an indication that the antisense oligomer targets sequences that are not in all of UBE3A transcripts, that are not accessible to the antisense oligomer for binding at a given moment in time, or both. For instance, an antisense oligomer provided herein that can achieve about 50% maximal UBE3A knockdown efficiency in a cell can be targeting a sequence that is only present in 50% of UBE3A transcripts in the cell. Alternatively or additionally, it can be because half of the time that the target site of the antisense oligomer on the UBE3A transcript is occupied by another protein complex or otherwise physically inaccessible to the antisense oligomer. Because abnormally reduced level of UBE3A protein can also lead to pathological conditions, proper dosing of antisense oligomer that reduces UBE3A transcripts can be desired for therapeutic applications without causing adverse effects by excessively reducing the level of UBE3A transcripts. With an antisense oligomer provided herein that has an intermediate level of knockdown efficiency, there can be a ceiling to the amount of UBE3A knockdown by the antisense oligomer. Such a ceiling to the knockdown amount of UBE3A can thus create a safety buffer that prevents the antisense oligomer from excessively reducing UBE3A transcripts when administered to a subject that is in need of reducing UBE3A level, for instance, a subject that has duplication, overexpression, or a gain-of-function mutation, of UBE3A gene, or increased activity or expression level of UBE3A protein.

Agents (e.g., antisense oligomers) or vectors provided herein can be admixed with pharmaceutically acceptable active or inert substances for the preparation of pharmaceutical compositions or formulations. An agent (e.g., antisense oligomer) targeted to a UBE3A nucleic acid can be utilized in pharmaceutical compositions by combining the agent with a suitable pharmaceutically acceptable diluent or carrier. A pharmaceutically acceptable diluent can include phosphate-buffered saline (PBS), artificial cerebrospinal fluid (aCSF), physiological saline, or any other suitable solutions.

In some cases, the compositions and methods provided herein relate to a vector encoding an agent provided herein. In some cases, the vector comprises a viral vector encoding the agent. In some cases, the viral vector comprises an adenoviral vector, adeno-associated viral (AAV) vector, lentiviral vector, Herpes Simplex Virus (HSV) viral vector, or retroviral vector.

Pharmaceutically acceptable salts are suitable for use in contact with the tissues of humans and lower animals without undue toxicity, irritation, allergic response, etc., and are commensurate with a reasonable benefit/risk ratio. (See, e.g., S. M. Berge, et al., J. Pharmaceutical Sciences, 66: 1-19 (1977), incorporated herein by reference for this purpose. The salts can be prepared in situ during the final isolation and purification of the compounds, or separately by reacting the free base form with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other documented methodologies such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

In some embodiments, the compositions are formulated into any of many possible dosage forms such as, but not limited to, tablets, capsules, gel capsules, liquid syrups, soft gels, suppositories, and enemas. In embodiments, the compositions are formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. In embodiments, a pharmaceutical formulation or composition of the present disclosure includes, but is not limited to, a solution, emulsion, microemulsion, foam or liposome-containing formulation (e.g., cationic or noncationic liposomes).

The pharmaceutical composition or formulation described herein may comprise one or more penetration enhancers, carriers, excipients or other active or inactive ingredients as appropriate and well known to those of skill in the art or described in the published literature. In embodiments, liposomes also include sterically stabilized liposomes, e.g., liposomes comprising one or more specialized lipids. These specialized lipids result in liposomes with enhanced circulation lifetimes. In embodiments, a sterically stabilized liposome comprises one or more glycolipids or is derivatized with one or more hydrophilic polymers, such as a polyethylene glycol (PEG) moiety. In some embodiments, a surfactant is included in the pharmaceutical formulation or compositions. The use of surfactants in drug products, formulations and emulsions is well known in the art. In embodiments, the present disclosure employs a penetration enhancer to effect the efficient delivery of the antisense oligonucleotide, e.g., to aid diffusion across cell membranes and/or enhance the permeability of a lipophilic drug. In some embodiments, the penetration enhancers are a surfactant, fatty acid, bile salt, chelating agent, or non-chelating nonsurfactant.

In some embodiments, the pharmaceutical formulation comprises multiple agents (e.g., antisense oligomers). In embodiments, the agent (e.g., antisense oligomer) or a vector encoding the agent is administered in combination with another drug or therapeutic agent.

Pharmaceutical compositions comprising antisense oligomers can encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense oligomers, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an antisense oligomer which are cleaved by endogenous nucleases within the body, to form the active antisense oligomer.

Antisense oligomers disclosed herein can be covalently linked to one or more moieties or conjugates which enhance the activity, cellular distribution or cellular uptake of the resulting antisense oligomers. Conjugate groups can include cholesterol moieties and lipid moieties. Additional conjugate groups can include carbohydrates, phospholipids, biotin, phenazine, folate, phenanthridine, anthraquinone, acridine, fluoresceins, rhodamines, coumarins, and dyes.

Antisense oligomers of the present disclosure can also be modified to have one or more stabilizing groups that are generally attached to one or both termini of antisense oligomers to enhance properties such as, for example, nuclease stability. Included in stabilizing groups are cap structures. These terminal modifications can protect the antisense oligomer having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures can include, for example, inverted deoxy abasic caps. Further 3′ and 5′-stabilizing groups that can be used to cap one or both ends of an antisense oligomer to impart nuclease stability can include those disclosed in WO 03/004602 published on Jan. 16, 2003.

Any of the compositions provided herein can be administered to an individual. “Individual” can be used interchangeably with “subject” or “patient.” An individual can be a mammal, for example, a human or animal such as a non-human primate, a rodent, a rabbit, a rat, a mouse, a horse, a donkey, a goat, a cat, a dog, a cow, a pig, or a sheep. In embodiments, the individual is a human. In embodiments, the individual is a fetus, an embryo, or a child. In some embodiments, the compositions provided herein are administered to a cell ex vivo.

In some embodiments, the compositions provided herein are administered to an individual as a method of treating a disease or disorder. In certain embodiments, the individual has a neurological disorder. In some cases, the individual has a disease or disorder associated with excessive expression level or activity level of UBE3A protein. In some cases, the disease or disorder the composition provided herein is applicable to relates to duplication, overexpression, or a gain-of-function mutation, of a UBE3A gene, for instance, a duplication of chromosome 15q11.2-q13.1, Dup15q syndrome.

Dupl5q syndrome is one of the most common genetic variants associated with autism spectrum disorder (ASD), which is associated with duplication of chromosome 15q11.2-q13.1. This chromosomal region includes the imprinted Prader-Willi/Angelman syndrome critical region (PWACR), ubiquitin protein ligase E3A (UBE3A), small nuclear ribonucleoprotein polypeptide N (SNRPN), and three GABAA receptor genes (GABRB3, GABRA5, and GABRG3). Dup15q syndrome can include two primary types of duplications of 1511.2-13.1: (1) an isodicentric chromosome 15 (idic(15)) that results in two additional maternally derived copies on a supernumerary chromosome that includes 15p and the proximal region of 15q11, most commonly leading to four copies of the region, or (2) an interstitial 15q duplication in which one extra copy of the 15q11.2-q13.1 region occurs on the same chromosome arm, typically resulting in three copies of the region, and has an overall milder phenotype. Duplication of 15q11.2-q13.1 confers a strong risk for autism spectrum disorder, epilepsy, and intellectual disability in the patients. Studies have shown that patients with Dup15q syndrome can exhibit a number of symptoms, including certain behavior profile, such as relative weakness in the areas of motor skills, facial expression, social smile, and reciprocal social interaction. Patients with Dup15q syndrome can also present a distinctive electroencephalography (EEG) signature in the form of high amplitude spontaneous beta frequency (12-30 Hz) oscillations.

In certain embodiments, provided herein are methods for prophylactically reducing UBE3A expression in an individual. Certain embodiments include treating an individual in need thereof by administering to an individual a therapeutically effective amount of an agent (e.g., an antisense oligomer), or a vector encoding the agent, which is targeted to a UBE3A nucleic acid.

In some embodiments, the individual has a genetic disease, such as any of the diseases described herein. In some embodiments, the individual is at risk of having a disease, such as any of the diseases described herein. In some embodiments, the individual is at increased risk of having a disease or disorder caused by insufficient amount of a protein or insufficient activity of a protein. If an individual is “at an increased risk” of having a disease or disorder caused insufficient amount of a protein or insufficient activity of a protein, the method involves preventative or prophylactic treatment. For example, an individual may be at an increased risk of having such a disease or disorder because of family history of the disease. Typically, individuals at an increased risk of having such a disease or disorder benefit from prophylactic treatment (e.g., by preventing or delaying the onset or progression of the disease or disorder). In embodiments, a fetus is treated in utero, e.g., by administering the agent (e.g., antisense oligomer) or a vector encoding the agent to the fetus directly or indirectly (e.g., via the mother).

In one embodiment, administration of a therapeutically effective amount of an antisense oligomer targeted to a UBE3A nucleic acid is accompanied by monitoring of UBE3A levels in an individual, to determine an individual's response to administration of the antisense oligomer. An individual's response to administration of the antisense oligomer can be used by a physician to determine the amount and duration of therapeutic intervention.

In certain embodiments, administration of an antisense oligomer targeted to a UBE3A nucleic acid results in reduction of the processed mRNA encoding the UBE3A protein (e.g., UBE3A mRNA) and or protein expression by at least 15, 20, 25, 30, 35, 40, 45, 50, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or a range defined by any two of these values.

In some embodiments, the level of the processed mRNA encoding the UBE3A protein (e.g., UBE3A mRNA) in the cell contacted with the agent or the vector is decreased by about 10% to about 99%, about 10% to about 95%, about 10% to about 90%, about 10% to about 80%, about 10% to about 70%, about 10% to about 60%, about 10% to about 50%, about 10% to about 40%, about 10% to about 30%, about 10% to about 20%, about 20% to about 90%, about 20% to about 80%, about 20% to about 70%, about 20% to about 60%, about 20% to about 50%, about 20% to about 40%, about 20% to about 30%, about 30% to about 80%, about 30% to about 70%, about 30% to about 60%, about 30% to about 50%, about 40% to about 70%, about 40% to about 60%, about 40% to about 50%, about 50% to about 99%, about 50% to about 95%, about 50% to about 90%, about 50% to about 80%, about 50% to about 70%, about 50% to about 60%, about 60% to about 99%, about 60% to about 95%, about 60% to about 90%, about 60% to about 80%, about 60% to about 70%, about 70% to about 99%, about 70% to about 95%, about 70% to about 90%, about 70% to about 80%, about 80% to about 99%, about 80% to about 95%, about 80% to about 90%, about 90% to about 99%, about 90% to about 95%, or about 95% to about 99%, as compared to an otherwise same cell not contacted with the agent or the vector.

In some embodiments, the level of the processed mRNA encoding the UBE3A protein (e.g., UBE3A mRNA) in the cell contacted with the agent or the vector is decreased by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%, as compared to an otherwise same cell not contacted with the agent or the vector.

In some cases, the method and composition provided herein reduces a level of the UBE3A protein in the cell. In some embodiments, the level of the UBE3A protein in the cell contacted with the agent or the vector is decreased by about 10% to about 99%, about 10% to about 95%, about 10% to about 90%, about 10% to about 80%, about 10% to about 70%, about 10% to about 60%, about 10% to about 50%, about 10% to about 40%, about 10% to about 30%, about 10% to about 20%, about 20% to about 90%, about 20% to about 80%, about 20% to about 70%, about 20% to about 60%, about 20% to about 50%, about 20% to about 40%, about 20% to about 30%, about 30% to about 80%, about 30% to about 70%, about 30% to about 60%, about 30% to about 50%, about 40% to about 70%, about 40% to about 60%, about 40% to about 50%, about 50% to about 99%, about 50% to about 95%, about 50% to about 90%, about 50% to about 80%, about 50% to about 70%, about 50% to about 60%, about 60% to about 99%, about 60% to about 95%, about 60% to about 90%, about 60% to about 80%, about 60% to about 70%, about 70% to about 99%, about 70% to about 95%, about 70% to about 90%, about 70% to about 80%, about 80% to about 99%, about 80% to about 95%, about 80% to about 90%, about 90% to about 99%, about 90% to about 95%, or about 95% to about 99%, as compared to an otherwise same cell not contacted with the agent or the vector. In some cases, the level of the UBE3A protein in the cell contacted with the agent or the vector is decreased by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%, as compared to an otherwise same cell not contacted with the agent or the vector.

In certain embodiments, administration of an antisense oligomer targeted to a UBE3A nucleic acid results in improved cognitive function in an animal. In certain embodiments, administration of a UBE3A antisense oligomer improves cognitive function by at least 15, 20, 25, 30, 35, 40, 45, 50, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or a range defined by any two of these values.

In certain embodiments, administration of an antisense oligomer targeted to a UBE3A nucleic acid results in improved motor function in an animal. In certain embodiments, administration of a UBE3A antisense oligomer improves cognitive function by at least 15, 20, 25, 30, 35, 40, 45, 50, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or a range defined by any two of these values.

In certain embodiments, administration of an antisense oligomer targeted to a UBE3A nucleic acid results in improved anxiety in an animal. In certain embodiments, administration of a UBE3A antisense oligomer improves anxiety by at least 15, 20, 25, 30, 35, 40, 45, 50, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or a range defined by any two of these values. In certain embodiments, administration of an antisense oligomer targeted to a UBE3A nucleic acid results in improved social interaction in an animal. In certain embodiments, administration of a UBE3A antisense oligomer improves social interaction by at least 15, 20, 25, 30, 35, 40, 45, 50, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or a range defined by any two of these values. In certain embodiments, administration of an antisense oligomer targeted to a UBE3A nucleic acid results in reduction of seizures. In certain embodiments, administration of a UBE3A antisense oligomer reduces seizures by at least 15, 20, 25, 30, 35, 40, 45, 50, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or a range defined by any two of these values. In certain embodiments, administration of an antisense oligomer targeted to a UBE3A nucleic acid results in normalized EEG discharges.

In certain embodiments, pharmaceutical compositions comprising an antisense oligomer targeted to UBE3A are used for the preparation of a medicament for treating a patient suffering or susceptible to a disorder including Dup15q syndrome.

Suitable routes for administration of agents (e.g., antisense oligomers) or a vector encoding the agent of the present disclosure can vary depending on cell type to which delivery of the agents or the vector is desired. The agent or vector encoding the agent of the present disclosure can be administered to patients parenterally, for example, by intrathecal injection, intracerebroventricular injection, intra cisterna magna injection, intraperitoneal injection, intramuscular injection, subcutaneous injection, or intravenous injection.

In embodiments, the antisense oligomer is administered with one or more agents capable of promoting penetration of the subject antisense oligomer across the blood-brain barrier by any method known in the art. For example, delivery of agents by administration of an adenovirus vector to motor neurons in muscle tissue is described in U.S. Pat. No. 6,632,427, incorporated herein by reference. Delivery of vectors directly to the brain, e.g., the striatum, the thalamus, the hippocampus, or the substantia nigra, is described, e.g., in U.S. Pat. No. 6,756,523, incorporated herein by reference.

In some embodiments, the antisense oligomers are linked or conjugated with agents that provide desirable pharmaceutical or pharmacodynamic properties. In embodiments, the antisense oligomer is coupled to a substance, known in the art to promote penetration or transport across the blood-brain barrier, e.g., an antibody to the transferrin receptor. In embodiments, the antisense oligonucleotide is linked with a viral vector, e.g., to render the antisense oligomer more effective or increase transport across the blood-brain barrier. In embodiments, osmotic blood brain barrier disruption is assisted by infusion of sugars, e.g., meso erythritol, xylitol, D(+) galactose, D(+) lactose, D(+) xylose, dulcitol, myo-inositol, L(−) fructose, D(−) mannitol, D(+) glucose, D(+) arabinose, D(−) arabinose, cellobiose, D(+) maltose, D(+) raffinose, L(+) rhamnose, D(+) melibiose, D(−) ribose, adonitol, D(+) arabitol, L(−) arabitol, D(+) fucose, L(−) fucose, D(−) lyxose, L(+) lyxose, and L(−) lyxose, or amino acids, e.g., glutamine, lysine, arginine, asparagine, aspartic acid, cysteine, glutamic acid, glycine, histidine, leucine, methionine, phenylalanine, proline, serine, threonine, tyrosine, valine, and taurine. Methods and materials for enhancing blood brain barrier penetration are described, e.g., in U.S. Pat. Nos. 9,193,969, 4,866,042, 6,294,520, and 6,936,589, each incorporated herein by reference.

In some embodiments, subjects treated using the methods and compositions are evaluated for improvement in condition using any methods known and described in the art.

The effects of the agent (e.g., antisense oligomer) provided herein on the level, activity or expression of UBE3A nucleic acids or UBE3A protein can be tested in vitro in a variety of cell types. Illustrative cell types include, but are not limited to, Hela cells, HSO2 cells, 293T cells, HepG2 cells, Hep3B cells, and primary hepatocytes.

In some aspects, provided herein are methods for treatment of cells with agents (e.g., antisense oligomers). Cells can be treated with agents (e.g., antisense oligomers) or a vector encoding the agent when the cells reach approximately 60-80% confluency in culture.

For instance, the antisense oligomer can be introduced into cultured cells with aid of cationic lipid transfection reagent LIPOFECTIN (Invitrogen, Carlsbad, CA). Antisense oligomers can be mixed with LIPOFECTIN in OPTI-MEM 1 (Invitrogen, Carlsbad, CA) to achieve the desired final concentration of antisense oligomer and a LIPOFECTIN concentration that may range from 2 to 12 μg/mL per 100 nM antisense oligonucleotide. The antisense oligomer can also be introduced into cultured cells with the aid of LIPOFECTAMINE (Invitrogen, Carlsbad, CA). Antisense oligomer can mixed with LIPOFECTAMINE in OPTI-MEM 1 reduced serum medium (Invitrogen, Carlsbad, CA) to achieve the desired concentration of antisense oligonucleotide and a LIPOFECTAMINE concentration that may range from 2 to 12 μg/mL per 100 nM antisense oligonucleotide. Another reagent that can be used to introduce antisense oligomers into cultured cells is TURBOFECT (Thermo Scientific, Carlsbad, CA).

In some cases, antisense oligomers are introduced into cultured cells via electroporation.

Antisense inhibition of UBE3A nucleic acids can be assessed by measuring UBE3A protein levels or UBE3A mRNA transcript levels. UBE3A mRNA transcript levels can be measured by routine techniques in the art, such as real time PCR. Protein levels of UBE3A can be evaluated or quantitated in a variety of ways well known in the art, such as immunoprecipitation, Western blot analysis (immunoblotting), enzyme-linked immunosorbent assay (ELISA), quantitative protein assays, protein activity assays (for example, caspase activity assays), immunohistochemistry, immunocytochemistry or fluorescence-activated cell sorting (FACS). Antibodies directed to a target can be identified and obtained from a variety of sources, such as the MSRS catalog of antibodies (Aerie Corporation, Birmingham, MI), or can be prepared via conventional monoclonal or polyclonal antibody generation methods well known in the art.

### In Vivo Testing of Antisense Oligomers

Antisense oligomers, for example, antisense oligonucleotides, are tested in animals to assess their ability to inhibit expression of UBE3A and produce phenotypic changes, such as, improved behavior, motor function, and cognition. In certain embodiments, motor function is measured by walking initiation analysis, rotarod, grip strength, pole climb, open field performance, balance beam, hind_paw footprint testing in the animal. In certain embodiments, behavior is measured by elevated plus maze and three-chamber social interaction. Testing can be performed in normal animals, or in experimental models. Following a period of treatment with antisense oligonucleotides, RNA can be isolated from CNS tissue or CSF and changes in UBE3A nucleic acid expression can be measured.

### Exemplary Embodiments

[1]A method of reducing expression of a UBE3A protein in a mammalian cell having duplication, overexpression, or a gain-of-function mutation, of a UBE3A gene that encodes the UBE3A protein, the method comprising contacting an agent or a vector encoding the agent to the mammalian cell, wherein the agent reduces a level of a processed mRNA encoding the UBE3A protein in the mammalian cell.

[2] The method of paragraph [1], wherein the agent comprises a polynucleotide sequence that is at least 80% complementary to at least 8 contiguous nucleic acids of the sequence set forth in any one of SEQ ID NO: 93-120.

[3] The method of paragraph [1], wherein the agent comprises a polynucleotide sequence that is at least 80% complementary to at least 8 contiguous nucleic acids of each mRNA transcript listed in Table 2.

[4] The method of any one of paragraphs [1]-[3], wherein the agent comprises an antisense oligomer.

[5] The method of paragraph [4], wherein the agent comprises an antisense oligomer with at least 80% identity to the sequence set forth in any one of SEQ ID NO: 1-92.

[6]A method of modulating expression of a UBE3A gene encoding a UBE3A protein in a mammalian cell, the method comprising contacting an agent or a vector encoding the agent to the mammalian cell, wherein the agent comprises a polynucleotide sequence that comprises an antisense oligomer with at least 80% identity to the sequence set forth in any one of SEQ ID NO: 1-92.

[7] The method of any one of paragraphs [4]-[6], wherein the antisense oligomer comprises a backbone modification, a modified sugar moiety or a combination thereof.

[8] The method of any one of paragraphs [4]-[7], wherein the antisense oligomer comprises a phosphorothioate linkage or a phosphorodiamidate linkage.

[9] The method of any one of paragraphs [4]-[8], wherein the antisense oligomer comprises a phosphorodiamidate morpholino, a locked nucleic acid, a peptide nucleic acid, a 2′-O-methyl moiety, a 2′-Fluoro moiety, a 2′-O-methoxyethyl moiety, or a 2′-NMA moiety.

[10] The method of any one of paragraphs [4]-[9], wherein the antisense oligomer comprises at least one modified sugar moiety.

[11] The method of any one of paragraphs [4]-[10], wherein the antisense oligomer comprises at least one, two, three, four, five, or six modified nucleosides at a 5′ end of the antisense oligomer.

[12] The method of any one of paragraphs [4]-[10], wherein the antisense oligomer comprises one, two, three, four, five, or six modified nucleosides at a 5′ end of the antisense oligomer.

[13] The method of any one of paragraphs [4]-[10], wherein the antisense oligomer comprises one, two, three, four, five, or six 2′-O-methoxyethyl modified nucleosides at a 5′ end of the antisense oligomer.

[14] The method of any one of paragraphs [4]-[13], wherein the antisense oligomer comprises at least one, two, three, four, five, or six modified nucleosides at a 3′ end of the antisense oligomer.

[15] The method of any one of paragraphs [4]-[13], wherein the antisense oligomer comprises one, two, three, four, five, or six modified nucleosides at a 3′ end of the antisense oligomer.

[16] The method of any one of paragraphs [4]-[13], wherein the antisense oligomer comprises one, two, three, four, five, or six 2′-O-methoxyethyl modified nucleosides at a 3′ end of the antisense oligomer.

[17] The method of any one of paragraphs [4]-[16], wherein the antisense oligomer comprises three, four, five, or six 2′-O-methoxyethyl modified nucleosides at a 5′ end of the antisense oligomer; three, four, five, or six 2′-O-methoxyethyl modified nucleosides at a 3′ end of the antisense oligomer; and a phosphorothioate linkage between any two neighboring nucleosides of the antisense oligomer.

[18] The method of any one of paragraphs [4]-[16], wherein the antisense oligomer comprises:
a 5′ region consisting of three, four, five, or six linked nucleosides;
a central region consisting of eight, nine, ten, eleven, or twelve linked nucleosides; and
a 3′ region consisting of three, four, five, or six linked nucleosides;
wherein each of the three, four, five, or six linked nucleosides in the 5′ region and each of three, four, five, or six linked nucleosides in the 3′ region comprise a modified sugar moiety, and wherein each of the eight, nine, ten, eleven, or twelve linked nucleosides in the central region is a deoxyribonucleoside.

[19] The method of any one of paragraphs [4]-[18], wherein the antisense oligomer consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 12 to 50 nucleobases, 12 to 40 nucleobases, 12 to 35 nucleobases, 12 to 30 nucleobases, 12 to 25 nucleobases, 12 to 20 nucleobases, 12 to 15 nucleobases, 15 to 50 nucleobases, 15 to 40 nucleobases, 15 to 35 nucleobases, 15 to 30 nucleobases, 15 to 25 nucleobases, 15 to 20 nucleobases, 15 to 19 nucleobases, 15 to 18 nucleobases, 15 to 16 nucleobases, 16 to 20 nucleobases, 16 to 19 nucleobases, 16 to 18 nucleobases, 17 to 20 nucleobases, 17 to 19 nucleobases, or 18 to 20 nucleobases.

[20] The method of any one of paragraphs [4]-[16], wherein the antisense oligomer is a modified oligonucleotide comprising the sequence set forth in any one of SEQ ID NO: 154-189 or 192-247.

[21] The method of any one of paragraphs [4]-[16], wherein the antisense oligomer is a modified oligonucleotide consisting of the sequence set forth in any one of SEQ ID NO: 154-189 or 192-247.

[22] The method of any one of paragraphs [1] to [6], wherein the vector comprises a viral vector encoding the agent.

[23] The method of paragraph [22], wherein the viral vector comprises an adenoviral vector, adeno-associated viral (AAV) vector, lentiviral vector, Herpes Simplex Virus (HSV) viral vector, or retroviral vector.

[24] The method of any one of paragraphs [4]-[23], wherein the antisense oligomer comprises a sequence with at least 90% identity to the sequence set forth in any one of SEQ ID NO: 1-92.

[25] The method of any one of paragraphs [4]-[23], wherein the antisense oligomer comprises a sequence with 100% identity to the sequence set forth in any one of SEQ ID NO: 1-92.

[26] The method of any one of paragraphs [4]-[23], wherein the antisense oligomer consists of a sequence with at least 80% identity to the sequence set forth in any one of SEQ ID NO: 1-92.

[27] The method of any one of paragraphs [4]-[23], wherein the antisense oligomer consists of a sequence with at least 90% identity to the sequence set forth in any one of SEQ ID NO: 1-92.

[28] The method of any one of paragraphs [4]-[23], wherein the antisense oligomer consists of a sequence with 100% identity to the sequence set forth in any one of SEQ ID NO: 1-92.

[29] The method of any one of paragraphs [6]-[28], wherein the method reduces a level of processed mRNA encoding the UBE3A protein in the mammalian cell.

[30] The method of any one of paragraphs [1]-[5] or [7]-[29], wherein the level of the processed mRNA encoding the UBE3A protein in the mammalian cell contacted with the agent or the vector is decreased by about 10% to about 99%, about 10% to about 95%, about 10% to about 90%, about 10% to about 80%, about 10% to about 70%, about 10% to about 60%, about 10% to about 50%, about 10% to about 40%, about 10% to about 30%, about 10% to about 20%, about 20% to about 90%, about 20% to about 80%, about 20% to about 70%, about 20% to about 60%, about 20% to about 50%, about 20% to about 40%, about 20% to about 30%, about 30% to about 80%, about 30% to about 70%, about 30% to about 60%, about 30% to about 50%, about 40% to about 70%, about 40% to about 60%, about 40% to about 50%, about 50% to about 99%, about 50% to about 95%, about 50% to about 90%, about 50% to about 80%, about 50% to about 70%, about 50% to about 60%, about 60% to about 99%, about 60% to about 95%, about 60% to about 90%, about 60% to about 80%, about 60% to about 70%, about 70% to about 99%, about 70% to about 95%, about 70% to about 90%, about 70% to about 80%, about 80% to about 99%, about 80% to about 95%, about 80% to about 90%, about 90% to about 99%, about 90% to about 95%, or about 95% to about 99%, as compared to an otherwise same cell not contacted with the agent or the vector.

[31] The method of any one of paragraphs [1]-[5] or [7]-[29], wherein the level of the processed mRNA encoding the UBE3A protein in the mammalian cell contacted with the agent or the vector is decreased by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%, as compared to an otherwise same mammalian cell not contacted with the agent or the vector.

[32] The method of any one of paragraphs [1]-[31], wherein the method reduces a level of the UBE3A protein in the mammalian cell.

[33] The method of paragraph [32], wherein the level of the UBE3A protein in the mammalian cell contacted with the agent or the vector is decreased by about 10% to about 99%, about 10% to about 95%, about 10% to about 90%, about 10% to about 80%, about 10% to about 70%, about 10% to about 60%, about 10% to about 50%, about 10% to about 40%, about 10% to about 30%, about 10% to about 20%, about 20% to about 90%, about 20% to about 80%, about 20% to about 70%, about 20% to about 60%, about 20% to about 50%, about 20% to about 40%, about 20% to about 30%, about 30% to about 80%, about 30% to about 70%, about 30% to about 60%, about 30% to about 50%, about 40% to about 70%, about 40% to about 60%, about 40% to about 50%, about 50% to about 99%, about 50% to about 95%, about 50% to about 90%, about 50% to about 80%, about 50% to about 70%, about 50% to about 60%, about 60% to about 99%, about 60% to about 95%, about 60% to about 90%, about 60% to about 80%, about 60% to about 70%, about 70% to about 99%, about 70% to about 95%, about 70% to about 90%, about 70% to about 80%, about 80% to about 99%, about 80% to about 95%, about 80% to about 90%, about 90% to about 99%, about 90% to about 95%, or about 95% to about 99%, as compared to an otherwise same cell not contacted with the agent or the vector.

[34] The method of paragraph [32], wherein the level of the UBE3A protein in the mammalian cell contacted with the agent or the vector is decreased by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%, as compared to an otherwise same cell not contacted with the agent or the vector.

[35] The method of any one of paragraphs [1]-[34], wherein the method comprises contacting the agent or the vector to a population of mammalian cells.

[36] The method of paragraph [35], wherein the agent reduces a level of the processed mRNA encoding the UBE3A protein in the population of mammalian cells by at most about 75%, at most about 70%, at most about 60%, at most about 50%, at most about 40%, at most about 30%, or at most about 20%, as compared to an otherwise same population of mammalian cells not contacted with the agent or the vector.

[37] The method of paragraph [35] or [36], wherein the agent reduces a level of the UBE3A protein in the population of mammalian cells by at most about 75%, at most about 70%, at most about 60%, at most about 50%, at most about 40%, at most about 30%, or at most about 20%, as compared to an otherwise same population of cells not contacted with the agent or the vector.

[38] The method of any one of paragraph [1] to [37], wherein the mammalian cell is ex vivo.

[39] The method of any one of paragraph [1] to [37], wherein the mammalian cell is in vivo.

[40] The method of any one of paragraph [1] to [39], wherein genome of the mammalian cell has a duplication of a genomic region that encompasses the UBE3A gene encoding the UBE3A protein.

[41] The method of any one of paragraph [1] to [39], wherein the mammalian cell is a human cell, and wherein genome of the mammalian cell has a duplication of chromosome 15q1.2-q13.1.

[42] The method of any one of paragraph [1] to [38], wherein the mammalian cell is obtained from a human subject suffering from Dup15q syndrome, or a progeny of a sample cell obtained from the human subject.

[43] An antisense oligomer that comprises a sequence with at least 80% identity to the sequence set forth in any one of SEQ ID NO: 1-92.

[44] The antisense oligomer of paragraph [43], wherein the antisense oligomer comprises a backbone modification, a modified sugar moiety or a combination thereof.

[45] The antisense oligomer of paragraph [43] or [44], wherein the antisense oligomer comprises a phosphorothioate linkage or a phosphorodiamidate linkage.

[46] The antisense oligomer of any one of paragraphs [43]-[45], wherein the antisense oligomer comprises a phosphorodiamidate morpholino, a locked nucleic acid, a peptide nucleic acid, a 2′-O-methyl moiety, a 2′-Fluoro moiety, a 2′-O-methoxyethyl moiety, or a 2′-NMA moiety.

[47] The antisense oligomer of any one of paragraphs [43]-[46], wherein the antisense oligomer comprises at least one modified sugar moiety.

[48] The antisense oligomer of any one of paragraphs [43]-[47], wherein the antisense oligomer comprises at least one, two, three, four, five, or six modified nucleosides at a 5′ end of the antisense oligomer.

[49] The antisense oligomer of any one of paragraphs [43]-[47], wherein the antisense oligomer comprises one, two, three, four, five, or six modified nucleosides at a 5′ end of the antisense oligomer.

[50] The antisense oligomer of any one of paragraphs [43]-[47], wherein the antisense oligomer comprises one, two, three, four, five, or six 2′-O-methoxyethyl modified nucleosides at a 5′ end of the antisense oligomer.

[51] The antisense oligomer of any one of paragraphs [43]-[50], wherein the antisense oligomer comprises at least one, two, three, four, five, or six modified nucleosides at a 3′ end of the antisense oligomer.

[52] The antisense oligomer of any one of paragraphs [43]-[50], wherein the antisense oligomer comprises one, two, three, four, five, or six modified nucleosides at a 3′ end of the antisense oligomer.

[53] The antisense oligomer of any one of paragraphs [43]-[50], wherein the antisense oligomer comprises one, two, three, four, five, or six 2′-O-methoxyethyl modified nucleosides at a 3′ end of the antisense oligomer.

[54] The antisense oligomer of any one of paragraphs [43]-[53], wherein the antisense oligomer comprises three, four, five, or six 2′-O-methoxyethyl modified nucleosides at a 5′ end of the antisense oligomer; three, four, five, or six 2′-O-methoxyethyl modified nucleosides at a 3′ end of the antisense oligomer; and a phosphorothioate linkage between any two neighboring nucleosides of the antisense oligomer.

[55] The antisense oligomer of any one of paragraphs [43]-[53], wherein the antisense oligomer comprises:
a 5′ region consisting of three, four, five, or six linked nucleosides;
a central region consisting of eight, nine, ten, eleven, or twelve linked nucleosides; and
a 3′ region consisting of three, four, five, or six linked nucleosides;
wherein each of the three, four, five, or six linked nucleosides in the 5′ region and each of three, four, five, or six linked nucleosides in the 3′ region comprise a modified sugar moiety, and wherein each of the eight, nine, ten, eleven, or twelve linked nucleosides in the central region is a deoxyribonucleoside.

[56] The antisense oligomer of any one of paragraphs [43]-[55], wherein the antisense oligomer consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 12 to 50 nucleobases, 12 to 40 nucleobases, 12 to 35 nucleobases, 12 to 30 nucleobases, 12 to 25 nucleobases, 12 to 20 nucleobases, 12 to 15 nucleobases, 15 to 50 nucleobases, 15 to 40 nucleobases, 15 to 35 nucleobases, 15 to 30 nucleobases, 15 to 25 nucleobases, 15 to 20 nucleobases, 15 to 19 nucleobases, 15 to 18 nucleobases, 15 to 16 nucleobases, 16 to 20 nucleobases, 16 to 19 nucleobases, 16 to 18 nucleobases, 17 to 20 nucleobases, 17 to 19 nucleobases, or 18 to 20 nucleobases.

[57] The antisense oligomer of any one of paragraphs [43]-[56], wherein the antisense oligomer comprises a sequence with at least 90% identity to the sequence set forth in any one of SEQ ID NO: 1-92.

[58] The antisense oligomer of any one of paragraphs [43]-[56], wherein the antisense oligomer comprises a sequence with 100% identity to the sequence set forth in any one of SEQ ID NO: 1-92.

[59] The antisense oligomer of any one of paragraphs [43]-[56], wherein the antisense oligomer consists of a sequence with at least 80% identity to the sequence set forth in any one of SEQ ID NO: 1-92.

[60] The antisense oligomer of any one of paragraphs [43]-[56], wherein the antisense oligomer consists of a sequence with at least 90% identity to the sequence set forth in any one of SEQ ID NO: 1-92.

[61] The antisense oligomer of any one of paragraphs [43]-[56], wherein the antisense oligomer consists of a sequence with 100% identity to the sequence set forth in any one of SEQ ID NO: 1-92.

[62] The antisense oligomer of any one of paragraphs [43]-[56], wherein the antisense oligomer is a modified oligonucleotide comprising the sequence set forth in any one of SEQ ID NO: 154-189 or 192-247.

[63] The antisense oligomer of any one of paragraphs [43]-[56], wherein the antisense oligomer is a modified oligonucleotide consisting of the sequence set forth in any one of SEQ ID NO: 154-189 or 192-247.

[64] The antisense oligomer of any one of paragraphs [43]-[63], wherein the antisense oligomer is configured to reduce a level of a processed mRNA transcript encoding a UBE3A protein in a population of mammalian cells upon contact with the population.

[65] The antisense oligomer of paragraph [64], wherein the antisense oligomer is configured to reduce the level of the processed mRNA encoding the UBE3A protein in the population by about 10% to about 99%, about 10% to about 95%, about 10% to about 90%, about 10% to about 80%, about 10% to about 70%, about 10% to about 60%, about 10% to about 50%, about 10% to about 40%, about 10% to about 30%, about 10% to about 20%, about 20% to about 90%, about 20% to about 80%, about 20% to about 70%, about 20% to about 60%, about 20% to about 50%, about 20% to about 40%, about 20% to about 30%, about 30% to about 80%, about 30% to about 70%, about 30% to about 60%, about 30% to about 50%, about 40% to about 70%, about 40% to about 60%, about 40% to about 50%, about 50% to about 99%, about 50% to about 95%, about 50% to about 90%, about 50% to about 80%, about 50% to about 70%, about 50% to about 60%, about 60% to about 99%, about 60% to about 95%, about 60% to about 90%, about 60% to about 80%, about 60% to about 70%, about 70% to about 99%, about 70% to about 95%, about 70% to about 90%, about 70% to about 80%, about 80% to about 99%, about 80% to about 95%, about 80% to about 90%, about 90% to about 99%, about 90% to about 95%, or about 95% to about 99%, as compared to an otherwise same population of mammalian cells not contacted with the antisense oligomer.

[66] The antisense oligomer of paragraph [64], wherein the antisense oligomer is configured to reduce the level of the processed mRNA encoding the UBE3A protein in the population by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%, as compared to an otherwise same population of mammalian cells not contacted with the antisense oligomer.

[67] The antisense oligomer of any one of paragraphs [64]-[66], wherein the antisense oligomer is configured to reduce the level of the processed mRNA encoding the UBE3A protein in the population by at most about 75%, at most about 70%, at most about 60%, at most about 50%, at most about 40%, at most about 30%, or at most about 20%, as compared to an otherwise same population of mammalian cells not contacted with the antisense oligomer.

[68] The antisense oligomer of any one of paragraphs [64]-[67], wherein the antisense oligomer is configured to reduce a level of the UBE3A protein in the population of mammalian cells.

[69] The antisense oligomer of paragraph [68], wherein the antisense oligomer is configured to reduce the level of the UBE3A protein in the population by about 10% to about 99%, about 10% to about 95%, about 10% to about 90%, about 10% to about 80%, about 10% to about 70%, about 10% to about 60%, about 10% to about 50%, about 10% to about 40%, about 10% to about 30%, about 10% to about 20%, about 20% to about 90%, about 20% to about 80%, about 20% to about 70%, about 20% to about 60%, about 20% to about 50%, about 20% to about 40%, about 20% to about 30%, about 30% to about 80%, about 30% to about 70%, about 30% to about 60%, about 30% to about 50%, about 40% to about 70%, about 40% to about 60%, about 40% to about 50%, about 50% to about 99%, about 50% to about 95%, about 50% to about 90%, about 50% to about 80%, about 50% to about 70%, about 50% to about 60%, about 60% to about 99%, about 60% to about 95%, about 60% to about 90%, about 60% to about 80%, about 60% to about 70%, about 70% to about 99%, about 70% to about 95%, about 70% to about 90%, about 70% to about 80%, about 80% to about 99%, about 80% to about 95%, about 80% to about 90%, about 90% to about 99%, about 90% to about 95%, or about 95% to about 99%, as compared to an otherwise same population of mammalian cells not contacted with the antisense oligomer.

[70] The antisense oligomer of paragraph [68], wherein the antisense oligomer is configured to reduce the level of the UBE3A protein in the population by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%, as compared to an otherwise same population of mammalian cells not contacted with the antisense oligomer.

[71] The antisense oligomer of any one of paragraphs [64]-[70], wherein the antisense oligomer is configured to reduce the level of the UBE3A protein in the population by at most about 75%, at most about 70%, at most about 60%, at most about 50%, at most about 40%, at most about 30%, or at most about 20%, as compared to an otherwise same population of mammalian cells not contacted with the antisense oligomer.

[72] The antisense oligomer of any one of paragraphs [64]-[71], wherein the mammalian cell is ex vivo.

[73] The antisense oligomer of any one of paragraphs [64]-[71], wherein the mammalian cell is in vivo.

[74] The antisense oligomer of any one of paragraphs [64]-[73], wherein genome of the mammalian cell has a duplication of a genomic region that encompasses the UBE3A gene encoding the UBE3A protein.

[75] The antisense oligomer of any one of paragraphs [64]-[74], wherein the mammalian cell is a human cell.

[76] The antisense oligomer of paragraph [75], wherein genome of the mammalian cell has a duplication of chromosome 15q11.2-q13.1.

[77] The antisense oligomer of any one of paragraphs [64]-[76], wherein the mammalian cell is obtained from a human subject suffering from Dupl5q syndrome, or a progeny of a sample cell obtained from the human subject.

[78]A pharmaceutical composition, comprising:
(a) a pharmaceutically acceptable excipient or carrier; and
(b) the antisense oligomer of any one of paragraphs [43]-[77].

[79]A pharmaceutical composition, comprising:
(a) a pharmaceutically acceptable excipient or carrier; and
(b) an agent or a vector encoding the agent, wherein the agent is configured to reduce a level of a processed mRNA transcript encoding a UBE3A protein in a mammalian cell upon contact with the mammalian cell.

[80] The pharmaceutical composition of paragraph [79], wherein the agent comprises a polynucleotide sequence that is at least 80% complementary to at least 8 contiguous nucleic acids of the sequence set forth in any one of SEQ ID NO: 93-120.

[81] The pharmaceutical composition of paragraph [79], wherein the agent comprises a polynucleotide sequence that is at least 80% complementary to at least 8 contiguous nucleic acids of each mRNA transcript listed in Table 2.

[82] The pharmaceutical composition of any one of paragraphs [79]-[81], wherein the agent comprises an antisense oligomer.

[83] The pharmaceutical composition of paragraph [82], wherein the antisense oligomer has at least 80% identity to the sequence set forth in any one of SEQ ID NO: 1-92.

[84] The pharmaceutical composition of any one of paragraphs [79]-[83], comprising the vector, and wherein the vector comprises a viral vector encoding the agent.

[85] The pharmaceutical composition of paragraph [84], wherein the viral vector comprises an adenoviral vector, adeno-associated viral (AAV) vector, lentiviral vector, Herpes Simplex Virus (HSV) viral vector, or retroviral vector.

[86] The pharmaceutical composition of any one of paragraphs [82]-[85], wherein the antisense oligomer comprises a sequence with at least 90% identity to the sequence set forth in any one of SEQ ID NO: 1-92.

[87] The pharmaceutical composition of any one of paragraphs [82]-[85], wherein the antisense oligomer comprises a sequence with 100% identity to the sequence set forth in any one of SEQ ID NO: 1-92.

[88] The pharmaceutical composition of any one of paragraphs [82]-[85], wherein the antisense oligomer consists of a sequence with at least 80% identity to the sequence set forth in any one of SEQ ID NO: 1-92.

[89] The pharmaceutical composition of any one of paragraphs [82]-[85], wherein the antisense oligomer consists of a sequence with at least 90% identity to the sequence set forth in any one of SEQ ID NO: 1-92.

[90] The pharmaceutical composition of any one of paragraphs [82]-[85], wherein the antisense oligomer consists of a sequence with 100% identity to the sequence set forth in any one of SEQ ID NO: 1-92.

[91] The pharmaceutical composition of paragraph [82], wherein the antisense oligomer is a modified oligonucleotide comprising the sequence set forth in any one of SEQ ID NO: 154-189 or 192-247.

[92] The pharmaceutical composition of paragraph [82], wherein the antisense oligomer is a modified oligonucleotide consisting of the sequence set forth in any one of SEQ ID NO: 154-189 or 192-247.

[93] The pharmaceutical composition of any one of paragraphs [79]-[92], wherein the agent is configured to reduce the level of the processed mRNA encoding the UBE3A protein in the population of the mammalian cells upon contact with the population.

[94] The pharmaceutical composition of paragraph [93], wherein the antisense oligomer is configured to reduce the level of the processed mRNA encoding the UBE3A protein in the population by about 10% to about 99%, about 10% to about 95%, about 10% to about 90%, about 10% to about 80%, about 10% to about 70%, about 10% to about 60%, about 10% to about 50%, about 10% to about 40%, about 10% to about 30%, about 10% to about 20%, about 20% to about 90%, about 20% to about 80%, about 20% to about 70%, about 20% to about 60%, about 20% to about 50%, about 20% to about 40%, about 20% to about 30%, about 30% to about 80%, about 30% to about 70%, about 30% to about 60%, about 30% to about 50%, about 40% to about 70%, about 40% to about 60%, about 40% to about 50%, about 50% to about 99%, about 50% to about 95%, about 50% to about 90%, about 50% to about 80%, about 50% to about 70%, about 50% to about 60%, about 60% to about 99%, about 60% to about 95%, about 60% to about 90%, about 60% to about 80%, about 60% to about 70%, about 70% to about 99%, about 70% to about 95%, about 70% to about 90%, about 70% to about 80%, about 80% to about 99%, about 80% to about 95%, about 80% to about 90%, about 90% to about 99%, about 90% to about 95%, or about 95% to about 99%, as compared to an otherwise same population of mammalian cells not contacted with the agent or the vector.

[95] The pharmaceutical composition of paragraph [93], wherein the agent is configured to reduce the level of the processed mRNA encoding the UBE3A protein in the population by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%, as compared to an otherwise same population of mammalian cells not contacted with the agent or the vector.

[96] The pharmaceutical composition of any one of paragraphs paragraph [93]-[95], wherein the agent is configured to reduce the level of the processed mRNA encoding the UBE3A protein in the population by at most about 75%, at most about 70%, at most about 60%, at most about 50%, at most about 40%, at most about 30%, or at most about 20%, as compared to an otherwise same population of mammalian cells not contacted with the antisense oligomer.

[97] The pharmaceutical composition of any one of paragraphs paragraph [93]-[96], wherein the agent is configured to reduce a level of the UBE3A protein in the population.

[98] The pharmaceutical composition of paragraph [97], wherein the agent is configured to reduce the level of the UBE3A protein in the population by about 10% to about 99%, about 10% to about 95%, about 10% to about 90%, about 10% to about 80%, about 10% to about 70%, about 10% to about 60%, about 10% to about 50%, about 10% to about 40%, about 10% to about 30%, about 10% to about 20%, about 20% to about 90%, about 20% to about 80%, about 20% to about 70%, about 20% to about 60%, about 20% to about 50%, about 20% to about 40%, about 20% to about 30%, about 30% to about 80%, about 30% to about 70%, about 30% to about 60%, about 30% to about 50%, about 40% to about 70%, about 40% to about 60%, about 40% to about 50%, about 50% to about 99%, about 50% to about 95%, about 50% to about 90%, about 50% to about 80%, about 50% to about 70%, about 50% to about 60%, about 60% to about 99%, about 60% to about 95%, about 60% to about 90%, about 60% to about 80%, about 60% to about 70%, about 70% to about 99%, about 70% to about 95%, about 70% to about 90%, about 70% to about 80%, about 80% to about 99%, about 80% to about 95%, about 80% to about 90%, about 90% to about 99%, about 90% to about 95%, or about 95% to about 99%, as compared to an otherwise same population of mammalian cells not contacted with the agent or the vector.

[99] The pharmaceutical composition of paragraph [97], wherein the agent is configured to reduce the level of the UBE3A protein in the population by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%, as compared to an otherwise same population of mammalian cells not contacted with the agent or the vector.

[100] The pharmaceutical composition of any one of paragraphs [97]-[99], wherein the agent is configured to reduce the level of the UBE3A protein in the population by at most about 75%, at most about 70%, at most about 60%, at most about 50%, at most about 40%, at most about 30%, or at most about 20%, as compared to an otherwise same population of mammalian cells not contacted with the antisense oligomer.

[101] The pharmaceutical composition of any one of paragraphs [79]-[100], wherein the mammalian cell is ex vivo.

[102] The pharmaceutical composition of any one of paragraphs [79]-[100], wherein the mammalian cell is in vivo.

[103] The pharmaceutical composition of any one of paragraphs [79]-[102], wherein genome of the mammalian cell has a duplication of a genomic region that encompasses the UBE3A gene encoding the UBE3A protein.

[104] The pharmaceutical composition of any one of paragraphs [79]-[103], wherein the mammalian cell is a human cell.

[105] The pharmaceutical composition of paragraph [104], wherein genome of the mammalian cell has a duplication of chromosome 15q11.2-q13.1.

[106] The pharmaceutical composition of any one of paragraphs [79]-[99], wherein the mammalian cell is obtained from a human subject suffering from Dup15q syndrome, or a progeny of a sample cell obtained from the human subject.

[107] The pharmaceutical composition of any one of paragraphs [78]-[106], wherein the pharmaceutical composition is formulated for intracerebroventricular injection, intraperitoneal injection, intramuscular injection, intrathecal injection, intra cisterna magna injection, subcutaneous injection, oral administration, synovial injection, intravitreal administration, subretinal injection, topical application, implantation, or intravenous injection.

[108] The pharmaceutical composition of any one of paragraphs [78]-[106], wherein the pharmaceutical composition is formulated for intrathecal injection.

[109] The pharmaceutical composition of any one of paragraphs [78]-[108], wherein the pharmaceutically acceptable excipient or carrier comprises artificial cerebrospinal fluid.

[110] The pharmaceutical composition of any one of paragraphs [78]-[109], wherein the pharmaceutical composition further comprises a second therapeutic agent.

[111] The pharmaceutical composition of paragraph [110], wherein the second therapeutic agent comprises a small molecule, an antisense oligomer, or a gene editing molecule.

[112]A method of treating or reducing the likelihood of developing a disease or condition in a subject in need thereof by reducing expression of a UBE3A protein in cells of the subject, comprising contacting to the cells of the subject the pharmaceutical composition of any one of paragraphs [78]-[111].

[113] The method of paragraph [112], wherein the disease or condition is associated with overexpression or gain-of-function mutation in a UBE3A gene encoding the UBE3A protein.

[114] The method of paragraph [112] or [113], wherein genomes of the cells of the subject have at least one excessive copy of a UBE3A gene encoding the UBE3A protein.

[115] The method of paragraph [112] or [114], wherein genomes of the cells of the subject have a duplication of a genomic region encompassing a UBE3A gene encoding the UBE3A protein.

[116] The method of paragraph [112] or [115], wherein genomes of the cells of the subject have a duplication of chromosome 15ql1.2-q13.1.

[117] The method of any one of paragraphs [112]-[116], wherein the disease or condition comprises Dup15q syndrome, autism spectrum disorder, epilepsy, or intellectual disability.

[118] The method of any one of paragraphs [112]-[117], wherein the subject is a human.

[119] The method of any one of paragraphs [112]-[118], wherein the subject is a fetus, an embryo, or a child.

[120] The method of any one of paragraphs [112]-[119], wherein the cells are ex vivo.

[121] The method of any one of paragraphs [112]-[119], comprising administering the pharmaceutical composition to the subject by intracerebroventricular injection, intraperitoneal injection, intramuscular injection, intrathecal injection, intra cisterna magna injection, subcutaneous injection, oral administration, synovial injection, intravitreal administration, subretinal injection, topical application, implantation, or intravenous injection.

[122] The method of any one of paragraphs [112]-[119], comprising administering the pharmaceutical composition to the subject by intrathecal injection.

[123] The method of any one of paragraphs [112]-[122], wherein the method treats the disease or condition.

### EXAMPLES

The present disclosure will be more specifically illustrated by the following Examples.

However, it should be understood that the present disclosure is not limited by these examples in any manner.

### Example 1: Knockdown of UBE3A Gene in Human Cells

In one experiment, effects on human UBE3A mRNA transcripts of compound molecules according to some embodiments of the present disclosure were examined. The compound molecules are modified oligonucleotides listed in Table 3. Among the examined compound molecules, there were 22 test oligonucleotides according to some embodiments of the present disclosure, one no template control (NTC) that serves as a negative control (“Ncontrol”), and one positive control oligonucleotide that is known for its knockdown effect on UBE3A gene (“Pcontrol”). As shown in the table, “/52MOErX/” represents a 2-O-methyoxyethyl modified ribonucleoside X at 5′ end of an oligonucleotide, where X is A, T, G, or methylated cytosine (MeC); “/i2MOErX/” represents an internal 2-O-methyoxyethyl modified ribonucleoside X, where X is A, T, G, or MeC); “/32MOErX/” represents a 2-O-methyoxyethyl modified ribonucleoside X at 3′ end of an oligonucleotide, where X is A, T, G, or MeC; other nucleosides A, T, G, and C are deoxyribonucleosides; “*” represents phosphorothioate linkage between two neighboring nucleosides.

TABLE 3
List of Compound Molecules

| Compound ID | Compound Sequence | SEQ ID NO. |
|---|---|---|
| Compound_1 | /52MOErC/*/i2MOErG/*/i2MOErC/*/i2MOErT/*/i2MO | 154 |
| | ErT/*C*A*T*T*C*G*G*C*T*A*/i2MOErG/*/i2MOErC/* | |
| | /i2MOErT/*/i2MOErT/*/32MOErC/ | |
| Compound_2 | /52MOErA/*/i2MOErT/*/i2MOErT/*/i2MOErC/*/i2MO | 155 |
| | ErG/*G*C*T*A*G*C*T*T*C*A*/i2MOErA/*/i2MOErT/* | |
| | /i2MOErG/*/i2MOErT/*/32MOErC/ | |
| Compound_3 | /52MOErT/*/i2MOErC/*/i2MOErG/*/i2MOErC/*/i2MO | 156 |
| | ErT/*T*C*A*T*T*C*G*G*C*T*/i2MOErA/*/i2MOErG/* | |
| | /i2MOErC/*/i2MOErT/*/32MOErT/ | |
| Compound_4 | /52MOErG/*/i2MOErC/*/i2MOErT/*/i2MOErT/*/i2MO | 157 |
| | ErC/*A*T*T*C*G*G*C*T*A*G*/i2MOErC/*/i2MOErT/* | |
| | /i2MOErT/*/i2MOErC/*/32MOErA/ | |
| Compound_5 | /52MOErT/*/i2MOErC/*/i2MOErG/*G*C*T*A*G*C*T*T | 158 |
| | *C*A*/i2MOErA/*/i2MOErT/*/i2MOErG/*/32MOErT/ | |
| Compound_6 | /52MOErC/*/i2MOErA/*/i2MOErT/*/i2MOErT/*/i2MO | 159 |
| | ErC/*G*G*C*T*A*G*C*T*T*C*/i2MOErA/*/i2MOErA/* | |
| | /i2MOErT/*/i2MOErG/*/32MOErT/ | |
| Compound_7 | /52MOErT/*/i2MOErC/*/i2MOErG/*/i2MOErG/*C*T*A | 160 |
| | *G*C*T*T*C*A*A*/i2MOErT/*/i2MOErG/*/i2MOErT/* | |
| | /32MOErC/ | |
| Compound_8 | /52MOErC/*/i2MOErC/*/i2MOErG/*/i2MOErG/*/i2MO | 161 |
| | ErA/*C*A*A*G*T*G*C*A*T*C*/i2MOErA/*/i2MOErT/* | |
| | /i2MOErC/*/i2MOErT/*/32MOErA/ | |
| Compound_9 | /52MOErC/*/i2MOErC/*/i2MOErG/*/i2MOErG/*A*C*A | 162 |
| | *A*G*T*G*C*A*T*/i2MOErC/*/i2MOErA/*/i2MOErT/* | |
| | /i2MOErC/*/32MOErT/ | |
| Compound_10 | /52MOErT/*/i2MOErC/*/i2MOErA/*/i2MOErC/*/i2MO | 163 |
| | ErA/*T*T*C*C*A*C*G*T*T*A*/i2MOErG/*/i2MOErG/* | |
| | /i2MOErT/*/i2MOErG/*/32MOErA/ | |
| Compound_11 | /52MOErC/*/i2MOErT/*/i2MOErT/*/i2MOErC/*T*G*G | 164 |
| | *T*C*T*G*A*A*T*/i2MOErA/*/i2MOErA/*/i2MOErG/* | |
| | /i2MOErT/*/32MOErA/ | |
| Compound_12 | /52MOErC/*/i2MOErG/*/i2MOErG/*/i2MOErA/*/i2MO | 165 |
| | ErC/*A*A*G*T*G*C*A*T*C*A*/i2MOErT/*/i2MOErC/* | |
| | /i2MOErT/*/i2MOErA/*/32MOErT/ | |
| Compound_13 | /52MOErT/*/i2MOErG/*/i2MOErT/*/i2MOErA/*/i2MO | 166 |
| | ErC/*A*T*G*C*G*A*A*T*T*C*/i2MOErT/*/i2MOErA/* | |
| | /i2MOErT/*/i2MOErT/*/32MOErG/ | |
| Compound_14 | /52MOErT/*/i2MOErC/*/i2MOErC/*/i2MOErA/*/i2MO | 167 |
| | ErT/*A*G*C*G*A*T*C*A*T*C*/i2MOET/*/i2MOErC/* | |
| | /i2MOET/*/i2MOErA/*/32MOErG/ | |
| Compound_15 | /52MOErT/*/i2MOErT/*/i2MOErC/*/i2MOErC/*/i2MO | 168 |
| | ErG/*G*C*T*T*C*C*A*C*A*T*/i2MOErA/*/i2MOErT/* | |
| | /i2MOErA/*/i2MOErA/*/32MOErG/ | |
| Compound_16 | /52MOErC/*/i2MOErA/*/i2MOErT/*/i2MOErT/*C*T*C | 169 |
| | *C*G*A*A*T*C*T*/i2MOErG/*/i2MOErG/*/i2MOErT/* | |
| | /32MOErC/ | |
| Compound_17 | /52MOErC/*/i2MOErC/*/i2MOErT/*/i2MOErT/*/i2MO | 170 |
| | ErC/*C*T*G*T*T*T*T*C*A*T*/i2MOErT/*/i2MOErT/* | |
| | /i2MOErG/*/i2MOErT/*/32MOErA/ | |
| Compound_18 | /52MOErC/*/i2MOErC/*/i2MOErT/*/i2MOErT/*/i2MO | 171 |
| | ErT/*C*T*G*T*G*T*C*T*G*G*/i2MOErG/*/i2MOErC/* | |
| | /i2MOErC/*/i2MOErA/*/32MOErT/ | |
| Compound_19 | /52MOErC/*/i2MOErT/*/i2MOErC/*/i2MOErT/*/i2MO | 172 |
| | ErT/*A*C*A*G*A*T*T*T*T*T*/i2MOErA/*/i2MOErA/* | |
| | /i2MOErC/*/i2MOErC/*/32MOErT/ | |
| Compound_20 | /52MOErT/*/i2MOErA/*/i2MOErG/*/i2MOErG/*/i2MO | 173 |
| | ErT/*A*A*C*C*T*T*T*C*T*G*/i2MOErT/*/i2MOErG/* | |
| | /i2MOErT/*/i2MOErC/*/32MOErT/ | |
| Compound_21 | /52MOErG/*/i2MOErT/*/i2MOErG/*/i2MOErA/*/i2MO | 174 |
| | ErA/*C*A*T*A*C*C*A*A*T*A*/i2MOErT/*/i2MOErC/* | |
| | /i2MOErT/*/i2MOErG/*/32MOErG/ | |
| Compound_22 | /52MOErG/*/i2MOErT/*/i2MOErA/*/i2MOErT/*G*A*G | 175 |
| | *A*T*G*T*A*G*G*/i2MOErT/*/i2MOErA/*/i2MOErA/* | |
| | /i2MOErC/*/32MOErC/ | |
| Compound_23 | /52MOErA/*/i2MOErC/*/i2MOErA/* | 176 |
| | /i2MOErG/*/i2MOErG/*T* T*G*T* C*A*C* A*C*C* | |
| | /i2MOErA/*/i2MOErG/*/i2MOErT/* | |
| | /i2MOErC/*/32MOErT/ | |
| Compound_24 | /52MOErA/*/i2MOErG/*/i2MOErT/*/i2MOErA/*/i2MO | 177 |
| | ErT/*G*A*G*A*T*G*T*A*G*G*/i2MOErT/*/i2MOErA/* | |
| | /i2MOErA/*/i2MOErC/*/32MOErC/ | |
| Compound_25 | /52MOErA/*/i2MOErG/*/i2MOErC/* | 178 |
| | /i2MOErT/*/i2MOErG/*T* G*G*C* C*A*T* T*C*G* | |
| | /i2MOErG/*/i2MOErT/*/i2MOErG/* | |
| | /i2MOErA/*/32MOErC/ | |
| Compound_26 | /52MOErC/*/i2MOErA/*/i2MOErA/*/i2MOErG/*T*A* | 179 |
| | T*G*A* G*A*T* G*T*/i2MOErA/* | |
| | /i2MOErG/*/i2MOErG/*/i2MOErT/*/32MOErA/ | |
| Compound_27 | /52MOErG/*/i2MOErA/*/i2MOErT/* | 180 |
| | /i2MOErA/*/i2MOErA/*G* T*G*G* T*T*T* T*C*G* | |
| | /i2MOErA/*/i2MOErC/*/i2MOErA/* | |
| | /i2MOErA/*/32MOErT/ | |
| Compound_28 | /52MOErC/*/i2MOErG/*/i2MOErG/* | 181 |
| | /i2MOErA/*/i2MOErC/*A* A*G*T* G*C*A* T*C*A* | |
| | /i2MOErT/*/i2MOErC/*/i2MOErT/* | |
| | /i2MOErA/*/32MOErT/ | |
| Compound_29 | /52MOErC/*/i2MOErC/*/i2MOErG/*/i2MOErG/*A*C* | 182 |
| | A*A*G* T*G*C* A*T*/i2MOErC/* | |
| | /i2MOErA/*/i2MOErT/*/i2MOErC/*/32MOErT/ | |
| Compound_30 | /52MOErG/*/i2MOErT/*/i2MOErA/*/i2MOErA/*C*A*C | 183 |
| | *T*T*T*C*A*C*G*/i2MOErC/*/i2MOErA/*/i2MOErA/* | |
| | /i2MOErA/*/32MOErA/ | |
| Compound_31 | /52MOErC/*/i2MOErA/*/i2MOErT/*/i2MOErT/*C*T* | 184 |
| | C*C*G* A*A*T* C*T*/i2MOErG/* /i2MOErG/ | |
| | */i2MOErT/*/32MOErC/ | |
| Compound_32 | /52MOErG/*/i2MOErG/*/i2MOErA/*/i2MOErC/*A*A* | 185 |
| | G*T*G* C*A*T* C*A*/i2MOErT/* | |
| | /i2MOErC/*/i2MOErT/*/i2MOErA/*/32MOErT/ | |
| Compound_33 | /52MOErT/*/i2MOErC/*/i2MOErC/* | 186 |
| | /i2MOErA/*/i2MOErT/*A* G*C*G* A*T*C* A*T*C* | |
| | /i2MOErT/*/i2MOErC/*/i2MOErT/* | |
| | /i2MOErA/*/32MOErG/ | |
| Compound_34 | /52MOErA/*/i2MOErA/*/i2MOErG/*/i2MOErC/*T*G* | 187 |
| | T*G*G* C*C*A* T*T*/i2MOErC/* | |
| | /i2MOErG/*/i2MOErG/*/32MOErT/ | |
| Compound_35 | /52MOErC/*/i2MOErC/*/i2MOErT/* | 188 |
| | /i2MOErT/*/i2MOErT/*C* T*G*T* G*T*C* T*G*G* | |
| | /i2MOErG/*/i2MOErC/*/i2MOErC/* | |
| | /i2MOErA/*/32MOErT/ | |
| Compound_36 | /52MOErC/*/i2MOErC/*/i2MOErT/* | 189 |
| | /i2MOErT/*/i2MOErC/*C* T*G*T* T*T*T* C*A*T* | |
| | /i2MOErT/*/i2MOErT/*/i2MOErG/* | |
| | /i2MOErT/*/32MOErA/ | |
| Ncontrol | /52MOErA/*/i2MOErT/*/i2MOErC/*/i2MOErA/*/i2MO | 190 |
| | ErG/*T*T*G*C*C*G*T*G*T*C*/i2MOErT/*/i2MOErT/* | |
| | /i2MOErT/*/i2MOErC/*/32MOErT/ | |
| Pcontrol | /52MOErT/*/i2MOErG/*/i2MOErA/*/i2MOErG/*/i2MO | 191 |
| | ErC/*T*A*T*C*A*C*C*T*A*T*/i2MOErC/*/i2MOErC/* | |
| | /i2MOErT/*/i2MOErT/*/32MOErG/ | |

TABLE 4
List of Compound Molecules

| Compound ID | Compound Sequences | SEQ ID NO. |
|---|---|---|
| Compound_37 | /52MOErA/*/i2MOErC/*/i2MOErA/* | 192 |
| | /i2MOErT/*/i2MOErT/*C* G*G*C* T*A*G* C*T*T* | |
| | /i2MOErC/*/i2MOErA/*/i2MOErA/* | |
| | /i2MOErT/*/32MOErG/ | |
| Compound_38 | /52MOErT/*/i2MOErT/*/i2MOErC/*/i2MOErG/*G*C* | 193 |
| | T*A*G* C*T*T* C*A*/i2MOErA/* | |
| | /i2MOErT/*/i2MOErG/*/i2MOErT/*/32MOErC/ | |
| Compound_39 | /52MOErT/*/i2MOErT/*/i2MOErC/*/i2MOErG/*G*C* | 194 |
| | T*A*G* C*T*T* C*A*/i2MOErA/* | |
| | /i2MOErG/*/i2MOErG/*/32MOErT/ | |
| Compound_40 | /52MOErC/*/i2MOErT/*/i2MOErT/*/i2MOErA/*C*A* | 195 |
| | T*T*C* G*G*C* T*A*/i2MOErG/* | |
| | /i2MOErC/*/i2MOErT/*/i2MOErT/*/32MOErC/ | |
| Compound_41 | /52MOErC/*/i2MOErT/*/i2MOErT/*/i2MOErA/*C*A* | 196 |
| | T*T*C* G*G*C* T*A*/i2MOErG/* | |
| | /i2MOErC/*/i2MOErT/*/32MOErT/ | |
| Compound_42 | /52MOErA/*/i2MOErT/*/i2MOErT/*/i2MOErC/*G*G* | 197 |
| | C*T*A* G*C*T* T*C*/i2MOErA/* | |
| | /i2MOErA/*/i2MOErT/*/i2MOErG/*/32MOErT/ | |
| Compound_43 | /52MOErT/*/i2MOErA/*/i2MOErC/*/i2MOErC/*A*T* | 198 |
| | A*T*T* T*C*G* C*C*/i2MOErA/* | |
| | /i2MOErA/*/i2MOErA/*/i2MOErC/*/32MOErT/ | |
| Compound_44 | /52MOErA/*/i2MOErC/*/i2MOErC/* | 199 |
| | /i2MOErT/*/i2MOErT/*T* C*T*G* T*G*T* C*T*G* | |
| | /i2MOErG/*/i2MOErG/*/i2MOErC/* | |
| | /i2MOErC/*/32MOErA/ | |
| Compound_45 | /52MOErT/*/i2MOErC/*/i2MOErA/*/i2MOErC/*A*T* | 200 |
| | T*C*C* A*C*G* T*T*/i2MOErA/* | |
| | /i2MOErG/*/i2MOErG/*/i2MOErT/*/32MOErG/ | |
| Compound_46 | /52MOErC/*/i2MOErG/*/i2MOErT/* | 201 |
| | /i2MOErG/*/i2MOErT/*C* T*T*T* C*T*G* T*G*T* | |
| | /i2MOErC/*/i2MOErT/*/i2MOErG/* | |
| | /i2MOErG/*/32MOErG/ | |
| Compound_47 | /52MOErC/*/i2MOErT/*/i2MOErT/* A*C*A* T*T*C* | 202 |
| | G*G*C* T*/i2MOErA/*/i2MOErG/* | |
| | /i2MOErC/*/32MOErT/ | |
| Compound_48 | /52MOErT/*/i2MOErG/*/i2MOErT/* | 203 |
| | /i2MOErC/*/i2MOErT/*T* T*C*T* G*T*G* T*C*T* | |
| | /i2MOErG/*/i2MOErG/*/i2MOErG/* | |
| | /i2MOErC/*/32MOErC/ | |
| Compound_49 | /52MOErC/*/i2MOErT/*/i2MOErT/* | 204 |
| | /i2MOErA/*/i2MOErC/*C* C*G*G* C*T*T* C*C*A* | |
| | /i2MOErC/*/i2MOErA/*/i2MOErT/* | |
| | /i2MOErA/*/32MOErT/ | |
| Compound_50 | /52MOErT/*/i2MOErT/*/i2MOErA/*/i2MOErC/*A*T* | 205 |
| | T*C*G* G*C*T* A*G*/i2MOErC/* | |
| | /i2MOET/*/i2MOErT/*/i2MOErC/*/32MOErA/ | |
| Compound_51 | /52MOErA/*/i2MOErC/*/i2MOErA/*/i2MOErT/*T*C* | 206 |
| | G*G*C* T*A*G* C*T*/i2MOErT/* | |
| | /i2MOErC/*/i2MOErA/*/i2MOErA/*/32MOErT/ | |
| Compound_52 | /52MOErT/*/i2MOErT/*/i2MOErT/* | 207 |
| | /i2MOErG/*/i2MOErT/*T* G*C*A* A*T*A* G*G*C* | |
| | /i2MOErT/*/i2MOErT/*/i2MOErG/* | |
| | /i2MOErA/*/32MOErC/ | |
| Compound_53 | /52MOErC/*/i2MOErA/*/i2MOErT/* | 208 |
| | /i2MOErA/*/i2MOErC/*C* A*T*A* T*T*T* C*G*C* | |
| | /i2MOErC/*/i2MOErA/*/i2MOErA/* | |
| | /i2MOErA/*/32MOErC/ | |
| Compound_54 | /52MOErC/*/i2MOErT/*/i2MOErT/* | 209 |
| | /i2MOErA/*/i2MOErC/*A* T*T*C* G*G*C* T*A*G* | |
| | /i2MOErC/*/i2MOErT/*/i2MOErT/* | |
| | /i2MOErC/*/32MOErA/ | |
| Compound_55 | /52MOErT/*/i2MOErC/*/i2MOErA/*/i2MOErC/*A*T* | 210 |
| | T*C*C* A*C*G* T*T*/i2MOErA/* | |
| | /i2MOErG/*/i2MOErG/*/32MOErT/ | |
| Compound_56 | /52MOErA/*/i2MOErT/*/i2MOErA/* | 211 |
| | /i2MOErC/*/i2MOErC/*A* T*A*T* T*T*C* G*C*C* | |
| | /i2MOErA/*/i2MOErA/*/i2MOErA/* | |
| | /i2MOErC/*/32MOErT/ | |
| Compound_57 | /52MOErC/*/i2MOErC/*/i2MOErG/* | 212 |
| | /i2MOErT/*/i2MOErG/*T* C*T*T* T*C*T* G*T*G* | |
| | /i2MOErT/*/i2MOErC/*/i2MOErT/* | |
| | /i2MOErG/*/32MOErG/ | |
| Compound_58 | /52MOErG/*/i2MOErT/*/i2MOErC/* | 213 |
| | /i2MOErT/*/i2MOErT/*T* C*T*G* T*G*T* C*T*G* | |
| | /i2MOErG/*/i2MOErG/*/i2MOErC/* | |
| | /i2MOErC/*/32MOErA/ | |
| Compound_59 | /52MOErA/*/i2MOErA/*/i2MOErC/* | 214 |
| | /i2MOErC/*/i2MOErT/*T* T*C*T* G*T*G* T*C*T* | |
| | /i2MOErG/*/i2MOErG/*/i2MOErG/* | |
| | /i2MOErC/*/32MOErC/ | |
| Compound_60 | /52MOErG/*/i2MOErT/*/i2MOErG/* | 215 |
| | /i2MOErT/*/i2MOErC/*T* T*T*C* T*G*T* G*T*C* | |
| | /i2MOErT/*/i2MOErG/*/i2MOErG/* | |
| | /i2MOErG/*/32MOErC/ | |
| Compound_61 | /52MOErG/*/i2MOErT/*/i2MOErA/* | 216 |
| | /i2MOErG/*/i2MOErG/*T* A*A*C* C*T*T* T*C*T* | |
| | /i2MOErG/*/i2MOErT/*/i2MOErG/* | |
| | /i2MOErT/*/32MOErC/ | |
| Compound_62 | /52MOErA/*/i2MOErC/*/i2MOErA/* | 217 |
| | /i2MOErT/*/i2MOErT/*C* C*A*C* G*T*T* A*G*G* | |
| | /i2MOErT/*/i2MOErG/*/i2MOErA/* | |
| | /i2MOErC/*/32MOErA/ | |
| Compound_63 | /52MOErG/*/i2MOErT/*/i2MOErT/*/i2MOErG/*C*A* | 218 |
| | A*T*A* G*G*C* T*T*/i2MOErG/* | |
| | /i2MOErA/*/i2MOErC/*/i2MOET/*/32MOErA/ | |
| Compound_64 | /52MOErC/*/i2MOErA/*/i2MOErG/*/i2MOErG/*T*T* | 219 |
| | G*T*C* A*C*A* C*C*/i2MOErA/* | |
| | /i2MOErG/*/i2MOErT/*/i2MOErC/*/32MOErT/ | |
| Compound_65 | /52MOErA/*/i2MOErC/*/i2MOErC/* | 220 |
| | /i2MOErA/*/i2MOErT/*A* T*T*T* C*G*C* C*A*A* | |
| | /i2MOErA/*/i2MOErC/*/i2MOErT/* | |
| | /i2MOErT/*/32MOErC/ | |
| Compound_66 | /52MOErC/*/i2MOErT/*/i2MOErT/* | 221 |
| | /i2MOErA/*/i2MOErC/*C* C*G*G* A*C*A* A*G*T* | |
| | /i2MOErG/*/i2MOErC/*/i2MOErA/* | |
| | /i2MOErT/*/32MOErC/ | |
| Compound_67 | /52MOErT/*/i2MOErT/*/i2MOErA/*/i2MOErC/*C*C* | 222 |
| | G*G*A* C*A*A* G*T*/i2MOErG/* | |
| | /i2MOErC/*/i2MOErA/*/i2MOErT/*/32MOErC/ | |
| Compound_68 | /52MOErG/*/i2MOErG/*/i2MOErC/*/i2MOErC/*A*T* | 223 |
| | T* C*G* G*T*G* A*C*/i2MOErA/* | |
| | /i2MOErT/*/i2MOErC/*/32MOErA/ | |
| Compound_69 | /52MOErT/*/i2MOErG/*/i2MOErA/* | 224 |
| | /i2MOErT/*/i2MOErC/*T* T*T*T* A*C*A* A*G*C* | |
| | /i2MOErT/*/i2MOErG/*/i2MOErT/* | |
| | /i2MOErG/*/32MOErG/ | |
| Compound_70 | /52MOErA/*/i2MOErG/*/i2MOErC/*/i2MOErA/*A*G* | 225 |
| | T*A*T* G*A*G* A*T*/i2MOErG/* | |
| | /i2MOErT/*/i2MOErA/*/i2MOErG/*/32MOErG/ | |
| Compound_71 | /52MOErA/*/i2MOErC/*/i2MOErC/*/i2MOErA/*T*A* | 226 |
| | T*T*T* C*G*C* C*A*/i2MOErA/* | |
| | /i2MOErA/*/i2MOErC/*/i2MOErT/*/32MOErT/ | |
| Compound_72 | /52MOErG/*/i2MOErG/*/i2MOErT/* | 227 |
| | /i2MOErA/*/i2MOErA/*C* C*T*T* T*C*T* G*T*G* | |
| | /i2MOErT/*/i2MOErC/*/i2MOErT/* | |
| | /i2MOErG/*/32MOErG/ | |
| Compound_73 | /52MOErA/*/i2MOErC/*/i2MOErA/*/i2MOErG/*G*T* | 228 |
| | T*G*T* C*A*C* A*C*/i2MOErC/* | |
| | /i2MOErA/*/i2MOErG/*/i2MOET/*/32MOErC/ | |
| Compound_74 | /52MOErG/*/i2MOErT/*/i2MOErT/*/i2MOErG/*C*A* | 229 |
| | A*T*A* G*G*C* T*T*/i2MOErG/* | |
| | /i2MOErA/*/i2MOErC/*/32MOErT/ | |
| Compound_75 | /52MOErT/*/i2MOErG/*/i2MOErT/* | 230 |
| | /i2MOErG/*/i2MOErA/*A* C*A*T* A*C*C* A*A*T* | |
| | /i2MOErA/*/i2MOErT/*/i2MOErC/* | |
| | /i2MOErT/*/32MOErG/ | |
| Compound_76 | /52MOErA/*/i2MOErC/*/i2MOErT/* | 231 |
| | /i2MOErG/*/i2MOErT/*A* C*A*T* G*C*G* A*A*T* | |
| | /i2MOErT/*/i2MOErC/*/i2MOErT/* | |
| | /i2MOErA/*/32MOErT/ | |
| Compound_77 | /52MOErT/*/i2MOErT/*/i2MOErA/* | 232 |
| | /i2MOErC/*/i2MOErC/*C* G*G*A* C*A*A* G*T*G* | |
| | /i2MOErC/*/i2MOErA/*/i2MOErT/* | |
| | /i2MOErC/*/32MOErA/ | |
| Compound_78 | /52MOErT/*/i2MOErG/*/i2MOErT/* | 233 |
| | /i2MOErA/*/i2MOErG/*G* T*A*A* C*C*T* T*T*C* | |
| | /i2MOErT/*/i2MOErG/*/i2MOErT/* | |
| | /i2MOErG/*/32MOErT/ | |
| Compound_79 | /52MOErT/*/i2MOErT/*/i2MOErT/* | 234 |
| | /i2MOErC/*/i2MOErC/*C* A*G*A* A*C*T* C*C*C* | |
| | /i2MOErT/*/i2MOErA/*/i2MOErA/* | |
| | /i2MOErT/*/32MOErC/ | |
| Compound_80 | /52MOErC/*/i2MOErA/*/i2MOErA/*/i2MOErG/*T*A* | 235 |
| | T*G*A* G*A*T* G*T*/i2MOErA/* | |
| | /i2MOErG/*/i2MOErG/*/32MOErT/ | |
| Compound_81 | /52MOErA/*/i2MOErA/*/i2MOErG/*/i2MOErC/*T*G* | 236 |
| | T*G*G* C*C*A* T*T*/i2MOErC/* | |
| | /i2MOErG/*/i2MOErG/*/i2MOErT/*/32MOErG/ | |
| Compound_82 | /52MOErG/*/i2MOErG/*/i2MOErC/* | 237 |
| | /i2MOErC/*/i2MOErA/*T* T*C*G* G*T*G* A*C*A* | |
| | /i2MOErT/*/i2MOErC/*/i2MOErA/* | |
| | /i2MOErG/*/32MOErG/ | |
| Compound_83 | /52MOErT/*/i2MOErG/*/i2MOErC/* A*A*T* A*G*G* | 238 |
| | C*T*T* G*/i2MOErA/*/i2MOErC/* | |
| | /i2MOErT/*/32MOErA/ | |
| Compound_84 | /52MOErG/*/i2MOErG/*/i2MOErC/*/i2MOErC/*A*T* | 239 |
| | T*C*G* G*T*G* A*C*/i2MOErA/* | |
| | /i2MOErT/*/i2MOErC/*/i2MOErA/*/32MOErG/ | |
| Compound_85 | /52MOErA/*/i2MOErG/*/i2MOErC/*/i2MOErT/*T*T* | 240 |
| | G*T*C* A*C*A* C*C*/i2MOErA/* | |
| | /i2MOErG/*/i2MOErT/*/i2MOErC/*/32MOErT/ | |
| Compound_86 | /52MOErT/*/i2MOErC/*/i2MOErA/*/i2MOErT/*T*C* | 241 |
| | T*C*C* G*A*A* T*C*/i2MOErT/* | |
| | /i2MOErG/*/i2MOErG/*/i2MOErT/*/32MOErC/ | |
| Compound_87 | /52MOErG/*/i2MOErT/*/i2MOErA/* | 242 |
| | /i2MOErA/*/i2MOErC/*A* C*T*T* T*C*A* C*G*C* | |
| | /i2MOErA/*/i2MOErA/*/i2MOErA/* | |
| | /i2MOErA/*/32MOErA/ | |
| Compound_88 | /52MOErA/*/i2MOErG/*/i2MOErG/* | 243 |
| | /i2MOErT/*/i2MOErA/*A* C*C*T* T*T*C* T*G*T* | |
| | /i2MOErG/*/i2MOErT/*/i2MOErC/* | |
| | /i2MOErT/*/32MOErG/ | |
| Compound_89 | /52MOErT/*/i2MOErT/*/i2MOErT/*/i2MOErG/*T*T*G | 244 |
| | *C*A*A*T*A*G*G*/i2MOErC/*/i2MOErT/*/i2MOErT/* | |
| | /i2MOErG/*/32MOErA/ | |
| Compound_90 | /52MOErA/*/i2MOErT/*/i2MOErT/*/i2MOErT/*/i2MO | 245 |
| | ErC/*G*C*C*A*A*A*C*T*T*C*/i2MOErT/*/i2MOErG/* | |
| | /i2MOErA/*/i2MOErG/*/32MOErG/ | |
| Compound_91 | /52MOErT/*/i2MOErC/*/i2MOErT/*/i2MOErC/*A*A*G | 246 |
| | *G*T*A*A*G*C*T*/i2MOErG/*/i2MOErA/*/i2MOErG/* | |
| | /i2MOErC/*/32MOErT/ | |
| Compound_92 | /52MOErT/*/i2MOErT/*/i2MOErC/*/i2MOErC/*/i2MO | 247 |
| | ErC/*A*G*A*A*C*T*C*C*C*T*/i2MOErA/*/i2MOErA/* | |
| | /i2MOErT/*/i2MOErC/*/32MOErA/ | |

The experiments were conducted with HS02 (primary human fibroblasts) in 96-well plates. 8000 cells/well were seeded in 96-well plates. 24 UBE3A targeting ASOs (22 exemplary ASOs+2 controls) were tested in an 8-point concentration-response curve (CRC) format, e.g., 8 different concentrations of each ASO were tested, and the response to the ASO treatment was measured and plotted against the different concentrations. Two control ASOs include a positive control to assess successful ASO delivery at 100 nM (“Pcontrol”) and a negative control, which is an ASO targeting a different gene TUG1 at 100 nM (“Ncontrol”). For each cell plate, there were also wells reserved to receive transfection reagents only (“TF only”) as another negative control for quality control (QC). All the ASOs were delivered by lipofectamine 2000 transfection (0.2 μl/well). The 22 exemplary ASOs were added to the cell medium at a concentration of 100 nM, 31.62 nM, 10 nM, 3.16 nM, 1.0 nM, 0.32 nM, 0.10 nM, or 0.032 nM.

Each ASO source plate was tested on duplicate cell plates in the same experiment; lysate from each cell well tested in duplicate were measured on the same qPCR plate.

Each cell well was treated with the respective ASOs for 24 hours before subject to nuclei counting and potency measurement. To count the number of nuclei of the cells that received the ASO treatment, 20 μl/well of Hoechst 33342 (50 μg/mL, nuclear staining) was added for 30 minutes. After staining, the cells were imaged by live-imaging on IN Cell Analyzer 2200 (GE Healthcare) with a 10× objective, 4 fields per well were captured, and automated analysis of the captured images was conducted with Columbus software (PerkinElmer).

After imaging, the cells were lysed using lysis solution with DNAse I (25 μl/well) and stop solution (2.5 μl/well) from TaqMan one-step Cells-to-Ct kit. The cell lysates were then subject to qPCR tests: 15% of the cell lysate from each well was added for qPCR reaction with TaqMan Master Mix from TaqMan one-step Cells-to-Ct kit.

For the TaqMan assays, Hs UBE3A assay 3 (FAM) was multiplexed with Hs HPRT1 (VIC). Upon completion of the TaqMan qPCT assay, calculations of the amount of UBE3A transcripts were done using the 2^{-ΔΔCt} C method, normalized to the measurement of internal reference HPRT1, and the changes in gene expression compared to control condition (100% expression) were then determined.

Cell loss in the lipofectamine-only condition (TF only, orange) is below 0% indicating some toxicity in the conditions transfected with ASOs. To account for those differences, data was normalized to TUG1 ASO negative CTRL per plate. Cell loss was below 20% for all the test ASOs except for 4 wells, indicating low toxicity in general.

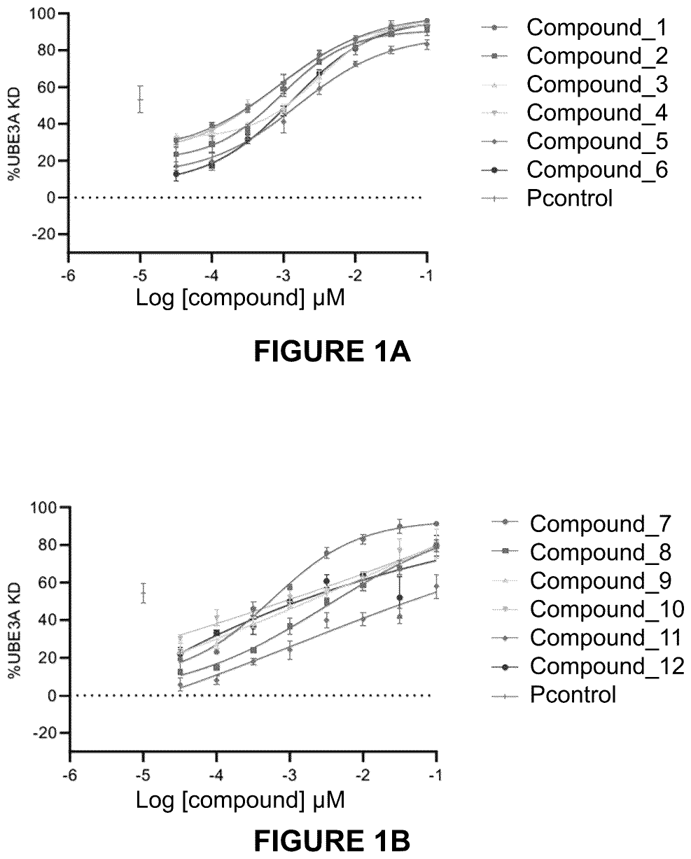

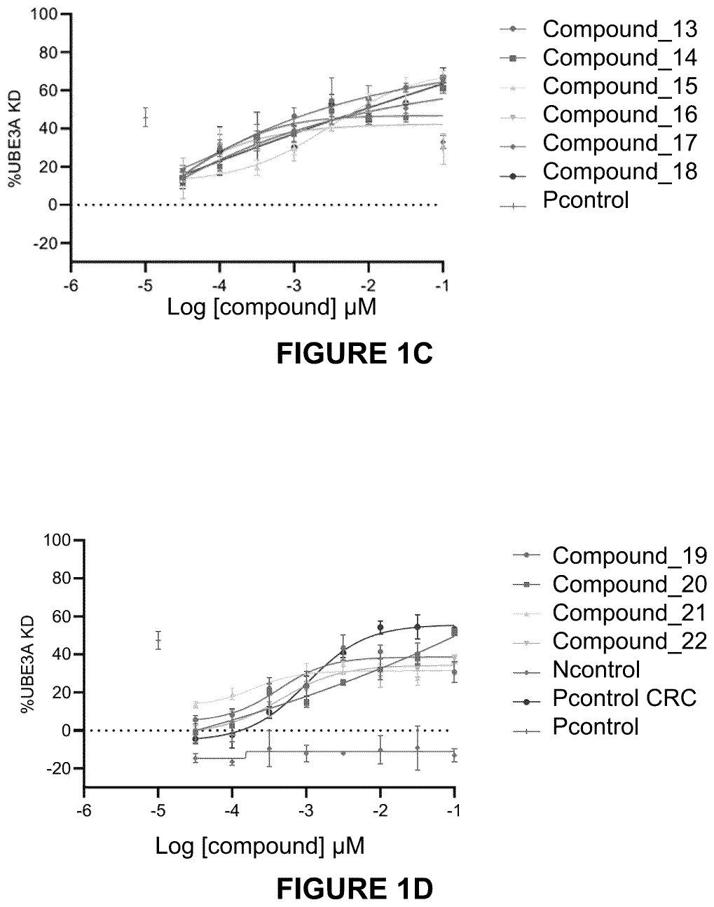

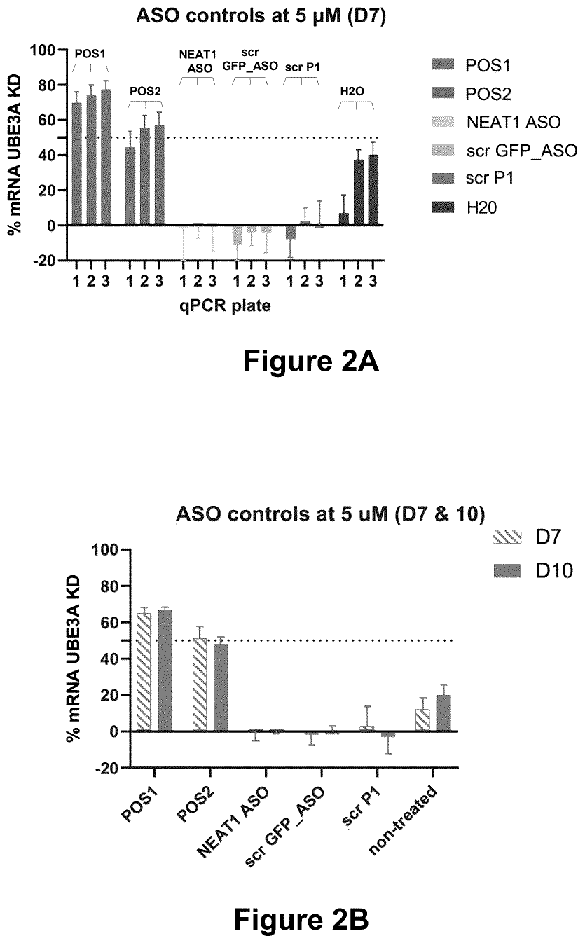

FIGS. 1A-ID show the CRCs for each tested ASOs, including the 22 exemplary ASOs, as well the potency (percent knockdown of UBE3A mRNA transcripts) of Pcontrol and Ncontrol.

Table 5 summarizes average UBE3A knockdown percentage for each of the tested ASOs, including Pcontrol and Ncontrol, and their respective EC50. EC50s (half maximal effective concentrations) were calculated using a non-linear regression (4 parameter) analysis. There were a number of ASOs (eight) for which EC50 values could not be calculated because their CRCs did not result in a S-curve. They are labeled as n.d. (not determined) in the table.

TABLE 5
Potency and EC50 of Tested ASOs

| | Ave UBE3A KD (%) | EC50 (nM) |
|---|---|---|
| Compound_1 | 96.2 | 0.92 |
| Compound_2 | 93.2 | 0.92 |
| Compound_3 | 95.5 | 3.06 |
| Compound_4 | 94.1 | 0.77 |
| Compound_5 | 87.4 | 1.57 |
| Compound_6 | 92.5 | 1.28 |
| Compound_7 | 91.1 | 0.57 |
| Compound_8 | 81.5 | 3.19 |
| Compound_9 | 81.9 | n.d. |
| Compound_10 | 79 | n.d. |
| Compound_11 | 68.9 | 1.39 |
| Compound_12 | 79.5 | n.d. |
| Compound_13 | 72 | n.d. |
| Compound_14 | 69.3 | n.d. |
| Compound_15 | 72.7 | 3.17 |
| Compound_18 | 71.1 | n.d. |
| Compound_20 | 57.2 | n.d. |
| Compound_21 | 40.5 | 0.19 |
| Compound_22 | 44.1 | 0.44 |
| Ncontrol | −7 | n.d. |
| Pcontrol | 59.3 | 1.03 |

SEQUENCE LISTING
SEQ ID NO: 1
CGCTTCATTCGGCTAGCTTC
SEQ ID NO: 2
ATTCGGCTAGCTTCAATGTC
SEQ ID NO: 3
TCGCTTCATTCGGCTAGCTT
SEQ ID NO: 4
GCTTCATTCGGCTAGCTTCA
SEQ ID NO: 5
TCGGCTAGCTTCAATGT
SEQ ID NO: 6
CATTCGGCTAGCTTCAATGT
SEQ ID NO: 7
TCGGCTAGCTTCAATGTC
SEQ ID NO: 8
CCGGACAAGTGCATCATCTA
SEQ ID NO: 9
CCGGACAAGTGCATCATCT
SEQ ID NO: 10
TCACATTCCACGTTAGGTGA
SEQ ID NO: 11
CTTCTGGTCTGAATAAGTA
SEQ ID NO: 12
CGGACAAGTGCATCATCTAT
SEQ ID NO: 13
TGTACATGCGAATTCTATTG
SEQ ID NO: 14
TCCATAGCGATCATCTCTAG
SEQ ID NO: 15
TTCCGGCTTCCACATATAAG
SEQ ID NO: 16
CATTCTCCGAATCTGGTC
SEQ ID NO: 17
CCTTCCTGTTTTCATTTGTA
SEQ ID NO: 18
CCTTTCTGTGTCTGGGCCAT
SEQ ID NO: 19
CTCTTACAGATTTTTAACCT
SEQ ID NO: 20
TAGGTAACCTTTCTGTGTCT
SEQ ID NO: 21
GTGAACATACCAATATCTGG
SEQ ID NO: 22
GTATGAGATGTAGGTAACC
SEQ ID NO: 23
ACAGGTTGTCACACCAGTCT
SEQ ID NO: 24
AGTATGAGATGTAGGTAACC
SEQ ID NO: 25
AGCTGTGGCCATTCGGTGAC
SEQ ID NO: 26
CAAGTATGAGATGTAGGTA
SEQ ID NO: 27
GATAAGTGGTTTTCGACAAT
SEQ ID NO: 28
CGGACAAGTGCATCATCTAT
SEQ ID NO: 29
CCGGACAAGTGCATCATCT
SEQ ID NO: 30
GTAACACTTTCACGCAAAA
SEQ ID NO: 31
CATTCTCCGAATCTGGTC
SEQ ID NO: 32
GGACAAGTGCATCATCTAT
SEQ ID NO: 33
TCCATAGCGATCATCTCTAG
SEQ ID NO: 34
AAGCTGTGGCCATTCGGT
SEQ ID NO: 35
CCTTTCTGTGTCTGGGCCAT
SEQ ID NO: 36
CCTTCCTGTTTTCATTTGTA
SEQ ID NO: 37
ACATTCGGCTAGCTTCAATG
SEQ ID NO: 38
TTCGGCTAGCTTCAATGTC
SEQ ID NO: 39
TTCGGCTAGCTTCAATGT
SEQ ID NO: 40
CTTACATTCGGCTAGCTTC
SEQ ID NO: 41
CTTACATTCGGCTAGCTT
SEQ ID NO: 42
ATTCGGCTAGCTTCAATGT
SEQ ID NO: 43
TACCATATTTCGCCAAACT
SEQ ID NO: 44
ACCTTTCTGTGTCTGGGCCA
SEQ ID NO: 45
TCACATTCCACGTTAGGTG
SEQ ID NO: 46
CGTGTCTTTCTGTGTCTGGG
SEQ ID NO: 47
CTTACATTCGGCTAGCT
SEQ ID NO: 48
TGTCTTTCTGTGTCTGGGCC
SEQ ID NO: 49
CTTACCCGGCTTCCACATAT
SEQ ID NO: 50
TTACATTCGGCTAGCTTCA
SEQ ID NO: 51
ACATTCGGCTAGCTTCAAT
SEQ ID NO: 52
TTTGTTGCAATAGGCTTGAC
SEQ ID NO: 53
CATACCATATTTCGCCAAAC
SEQ ID NO: 54
CTTACATTCGGCTAGCTTCA
SEQ ID NO: 55
TCACATTCCACGTTAGGT
SEQ ID NO: 56
ATACCATATTTCGCCAAACT
SEQ ID NO: 57
CCGTGTCTTTCTGTGTCTGG
SEQ ID NO: 58
GTCTTTCTGTGTCTGGGCCA
SEQ ID NO: 59
AACCTTTCTGTGTCTGGGCC
SEQ ID NO: 60
GTGTCTTTCTGTGTCTGGGC
SEQ ID NO: 61
GTAGGTAACCTTTCTGTGTC
SEQ ID NO: 62
ACATTCCACGTTAGGTGACA
SEQ ID NO: 63
GTTGCAATAGGCTTGACTA
SEQ ID NO: 64
CAGGTTGTCACACCAGTCT
SEQ ID NO: 65
ACCATATTTCGCCAAACTTC
SEQ ID NO: 66
CTTACCCGGACAAGTGCATC
SEQ ID NO: 67
TTACCCGGACAAGTGCATC
SEQ ID NO: 68
GGCCATTCGGTGACATCA
SEQ ID NO: 69
TGATCTTTTACAAGCTGTGG
SEQ ID NO: 70
AGCAAGTATGAGATGTAGG
SEQ ID NO: 71
ACCATATTTCGCCAAACTT
SEQ ID NO: 72
GGTAACCTTTCTGTGTCTGG
SEQ ID NO: 73
ACAGGTTGTCACACCAGTC
SEQ ID NO: 74
GTTGCAATAGGCTTGACT
SEQ ID NO: 75
TGTGAACATACCAATATCTG
SEQ ID NO: 76
ACTGTACATGCGAATTCTAT
SEQ ID NO: 77
TTACCCGGACAAGTGCATCA
SEQ ID NO: 78
TGTAGGTAACCTTTCTGTGT
SEQ ID NO: 79
TTTCCCAGAACTCCCTAATC
SEQ ID NO: 80
CAAGTATGAGATGTAGGT
SEQ ID NO: 81
AAGCTGTGGCCATTCGGTG
SEQ ID NO: 82
GGCCATTCGGTGACATCAGG
SEQ ID NO: 83
TGCAATAGGCTTGACTA
SEQ ID NO: 84
GGCCATTCGGTGACATCAG
SEQ ID NO: 85
AGCTGTGGCCATTCGGTGA
SEQ ID NO: 86
TCATTCTCCGAATCTGGTC
SEQ ID NO: 87
GTAACACTTTCACGCAAAAA
SEQ ID NO: 88
AGGTAACCTTTCTGTGTCTG
SEQ ID NO: 89
TTTGTTGCAATAGGCTTGA
SEQ ID NO: 90
ATTTCGCCAAACTTCTGAGG
SEQ ID NO: 91
TCTCAAGGTAAGCTGAGCT
SEQ ID NO: 92
TTCCCAGAACTCCCTAATCA

### Example 2: In Vivo ASO Tolerance of Antisense Oligonucleotides (ASOs) Via Intracerebroventricular (ICV) Administration

This example illustrates experiments that were conducted to test the in vivo tolerability of certain exemplary ASOs of the present disclosure in mice.

Effects of the exemplary ASOs were examined by in vivo experiments via intracerebroventricular (ICV) administration into mice. Adult C57BL/6 mice were surgically implanted with an ICV cannula. Following recovery from surgery, mice received an ICV injection of ASO or Dulbecco's phosphate buffered saline (DPBS) on day 0. Observations were made pre-dose and 1 hour, 2 hours, 4 hours, 24 hours, and 7 days after dosing. Animals were terminated 2 weeks post-dose. Terminal plasma, brain, liver, heart, and kidney tissues were collected and stored.

Adult male C57Bl/6 mice were used for this study. Upon arrival, animals were housed one per cage in polycarbonate cages and acclimated for at least 4 days prior to commencing the study. Animals were housed on a 12/12 hr light/dark cycle with room temperature (22+2° C.) and humidity (˜50%) maintained. The animals had access to food and water ad libitum. Experiments were conducted in accordance with protocols approved by the Institutional Animal Care and Use Committee of Charles River Laboratories South San Francisco.

Dosing formulations were prepared at appropriate concentrations to meet target dose levels in each mouse (e.g., 400 μg/mouse, 500 ag/mouse). All ASOs were formulated with DPBS and stored at −80° C. until the day of dosing. Each animal test condition contained six animals and each animal received one dose of intracerebroventricular injection constituting 5 μl of a test sample according to the groupings presented in Table 6, with the exceptions of Groups 4, 14, and 25, which were dropped from the study due to solubility issues. Group 1 was the control condition in which only DPBS was administered, and Group 2 was the positive control in which Tominersen, a known antisense oligonucleotide treatment for reduction of huntingtin protein (HTT) and mutated huntingtin protein (mHTT) expression levels, was administered. Groups 3-26 were the experimental conditions using the ASOs named.

TABLE 6
Animal Testing Groups and Conditions

| | | | | Injection | | | |
|---|---|---|---|---|---|---|---|
| | Test Article | | Route of | volume | Concentration | Group | Dose/ |
| Group | (TA) | Dose | administration | (μl) | (mg/ml) | size | animal |
| 1 | Vehicle Control | NA/mouse | ICV | 5 | N/A | 6 | 1 |
| | (DPBS) | | | | | | |
| 2 | Tominersen | 400 | ICV | 5 | 80 | 6 | 1 |
| | | μg/mouse | | | | | |
| 3 | Compound_23 | 400 | ICV | 5 | 80 | 6 | 1 |
| | | μg/mouse | | | | | |
| 4* | Compound_22 | N/A | N/A | N/A | N/A | N/A | 0 |
| 5 | Compound_25 | 400 | ICV | 5 | 80 | 6 | 1 |
| | | μg/mouse | | | | | |
| 6 | Compound_26 | 400 | ICV | 5 | 80 | 6 | 1 |
| | | μg/mouse | | | | | |
| 7 | Compound_27 | 500 | ICV | 5 | 100 | 6 | 1 |
| | | μg/mouse | | | | | |
| 8 | Compound_12 | 400 | ICV | 5 | 80 | 6 | 1 |
| | | μg/mouse | | | | | |
| 9 | Compound_89 | 400 | ICV | 5 | 80 | 6 | 1 |
| | | μg/mouse | | | | | |
| 10 | Compound_29 | 500 | ICV | 5 | 100 | 6 | 1 |
| | | μg/mouse | | | | | |
| 11 | Compound_11 | 500 | ICV | 5 | 100 | 6 | 1 |
| | | μg/mouse | | | | | |
| 12 | Compound_5 | 500 | ICV | 5 | 100 | 6 | 1 |
| | | μg/mouse | | | | | |
| 13 | Compound_31 | 500 | ICV | 5 | 100 | 6 | 1 |
| | | μg/mouse | | | | | |
| 14* | Compound_32 | N/A | N/A | N/A | N/A | N/A | 0 |
| 15 | Compound_2 | 500 | ICV | 5 | 100 | 6 | 1 |
| | | μg/mouse | | | | | |
| 16 | Compound_14 | 500 | ICV | 5 | 100 | 6 | 1 |
| | | μg/mouse | | | | | |
| 17 | Compound_90 | 500 | ICV | 5 | 100 | 6 | 1 |
| | | μg/mouse | | | | | |
| 18 | Compound_91 | 500 | ICV | 5 | 100 | 6 | 1 |
| | | μg/mouse | | | | | |
| 19 | Compound_34 | 500 | ICV | 5 | 100 | 6 | 1 |
| | | μg/mouse | | | | | |
| 20 | Compound_92 | 500 | ICV | 5 | 100 | 6 | 1 |
| | | μg/mouse | | | | | |
| 21 | Compound_18 | 500 | ICV | 5 | 100 | 6 | 1 |
| | | μg/mouse | | | | | |
| 22 | Compound_20 | 500 | ICV | 5 | 100 | 6 | 1 |
| | | μg/mouse | | | | | |
| 23 | Compound_15 | 500 | ICV | 5 | 100 | 6 | 1 |
| | | μg/mouse | | | | | |
| 24 | Compound_4 | 500 | ICV | 5 | 100 | 6 | 1 |
| | | μg/mouse | | | | | |
| 25* | Compound_21 | N/A | N/A | N/A | N/A | N/A | 0 |
| 26 | Compound_17 | 500 | ICV | 5 | 100 | 6 | 1 |
| | | μg/mouse | | | | | |

### Surgical Procedures

### ICV Cannulation Surgery

Mice were anesthetized using isoflurane (2%, 800 mi/mmn O₂). Bupivacaine was used for local analgesia and carprofen was used for peni-/post-operative analgesia. The animals were placed in a stereotaxic frame (KOPF® Laboratory Instruments, USA). Surgery was performed using aseptic techniques. Anterior-Posterior (AP), Medial-Lateral (ML), and Dorsal-Ventral (DV) axes were zeroed on Bregma. The following coordinates were used for the ICV infusion cannula (PLASTICS ONE®, Roanoke, Virginia): AP −0.3 mm, lateral −1.0, DV −2.2 mm from dura. The locations of the burr holes were designated and drilled. The drill was only used to penetrate the bone, and the infusion cannula was lowered into the lateral ventricle. Two screws and dental acrylic were used to secure the ICV cannula. After surgery, animals were provided food and water ad libitum. Any animal exhibiting unusual or adverse signs were not used in the study.

### Pre-And Post-Operative Care

Carprofen (5 mg/kg SC) was administered before the surgery and Carprofen (0.067 mg/ml) was provided in the drinking water to animals for 3 days after surgery. Animals were carefully monitored on the day of surgery and once daily for an additional 3 days after surgery.

### ICV Infusion (Day 0):

At least 7 days after surgery, animals were dosed according to the conditions presented in Table 7. Animals were dosed while they were conscious. Infusion cannulas were placed into the ICV cannula, and the test article was infused at a rate of 0.08 μl/sec. Infusion cannulas were left in the guide cannula for an additional 30 seconds prior to removal and replacement of the dummy stylet.

TABLE 7
ASO Dosing Regimen and Infusion Conditions via ICV

| | | | | | | Injec- |
|---|---|---|---|---|---|---|
| | | | | Vol- | | tion |
| | | | Dosing | ume | Rate of | Time |
| Group | N | Test Article | Regimen | (μl) | Infusion | (sec) |
| 1 | 6 | Vehicle | ICV, day 0 | 5 | 0.08 μl/sec | 62.5 |
| | | Control (DPBS) | | | | |
| 2 | 6 | Tominersen | ICV, day 0 | 5 | 0.08 μl/sec | 62.5 |
| 3 | 6 | Compound_23 | ICV, day 0 | 5 | 0.08 μl/sec | 62.5 |
| 5 | 6 | Compound_25 | ICV, day 0 | 5 | 0.08 μl/sec | 62.5 |
| 6 | 6 | Compound_26 | ICV, day 0 | 5 | 0.08 μl/sec | 62.5 |
| 7 | 6 | Compound_27 | ICV, day 0 | 5 | 0.08 μl/sec | 62.5 |
| 8 | 6 | Compound_12 | ICV, day 0 | 5 | 0.08 μl/sec | 62.5 |
| 9 | 6 | Compound_89 | ICV, day 0 | 5 | 0.08 μl/sec | 62.5 |
| 10 | 6 | Compound_9 | ICV, day 0 | 5 | 0.08 μl/sec | 62.5 |
| 11 | 6 | Compound_11 | ICV, day 0 | 5 | 0.08 μl/sec | 62.5 |
| 12 | 6 | Compound_5 | ICV, day 0 | 5 | 0.08 μl/sec | 62.5 |
| 13 | 6 | Compound_31 | ICV, day 0 | 5 | 0.08 μl/sec | 62.5 |
| 15 | 6 | Compound_2 | ICV, day 0 | 5 | 0.08 μl/sec | 62.5 |
| 16 | 6 | Compound_14 | ICV, day 0 | 5 | 0.08 μl/sec | 62.5 |
| 17 | 6 | Compound_90 | ICV, day 0 | 5 | 0.08 μl/sec | 62.5 |
| 18 | 6 | Compound_91 | ICV, day 0 | 5 | 0.08 μl/sec | 62.5 |
| 19 | 6 | Compound_34 | ICV, day 0 | 5 | 0.08 μl/sec | 62.5 |
| 20 | 6 | Compound_92 | ICV, day 0 | 5 | 0.08 μl/sec | 62.5 |
| 21 | 6 | Compound_18 | ICV, day 0 | 5 | 0.08 μl/sec | 62.5 |
| 22 | 6 | Compound_20 | ICV, day 0 | 5 | 0.08 μl/sec | 62.5 |
| 23 | 6 | Compound_15 | ICV, day 0 | 5 | 0.08 μl/sec | 62.5 |
| 24 | 6 | Compound_4 | ICV, day 0 | 5 | 0.08 μl/sec | 62.5 |
| 26 | 6 | Compound_17 | ICV, day 0 | 5 | 0.08 μl/sec | 62.5 |

### Behavioral Observations

On the day of dosing, animals were observed and scored pre-dose, at 1 hour, 2 hours, 4 hours, 24 hours, and 1 week post-dose according to Table 8. Detailed clinical observations were noted, including gait and other hindlimb deficiencies. Adverse events included ataxia, seizure, gait or coordination changes and weight loss (15% or more).

TABLE 8
Behavioral Scoring Criteria

| Behavior | Code | Score | Description |
|---|---|---|---|
| Spontaneous Activity | A | 1 | Not moving spontaneously |
| | | 2 | Minimal spontaneous movement (1-3 steps at a time) and/or severely sluggish |
| | | 3 | Reduced time moving and/or very slow |
| | | 4 | Slowed movement, but time moving normal |
| | | 5 | Hyperactive/jumping |
| Reactivity | R | 1 | Hyporeactive (needs multiple and/or forceful touch to react) |
| | | 2 | Hyperreactive (jumping, scratching, running on contact) |
| Convulsion | C | 1 | Stereotypic behavior (chewing, head flicks, head switching) |
| | | 2 | Isolated hindlimb or forelimb clonus |
| | | 3 | Isolated generalized convulsions |
| | | 4 | Consistent or frequent convulsions |
| Motor | M | 1 | Unsteady or uncoordinated movements |
| | | 2 | Frequently tripping and/or falling while walking |
| | | 3 | Dragging part or all of body while walking |
| | | 4 | Unable to traverse |
| Breathing | B | 1 | Slow Breathing (significantly <~2-3 breaths per second at rest) |
| | | 2 | Rapid Breathing (significantly >~2-3 breaths per second at rest) |
| Physical | H | | Hunched posture |
| | L | | Lowered pelvic height |
| | D | | Disheveled coat |
| | P | | Piloerection |
| | E | | Ptosis |
| | T | | Tremor/shaky at rest |
| | HT | | Head tilt |
| | X | | Unable to right self within 5 seconds |

### Humane Endpoints Post-dose

After dosing, animals were euthanized before the scheduled euthanasia timepoint if they met the criteria presented in Table 9.

TABLE 9
Euthanasia Timepoints upon Observation of Animal Behavior

| Timeframe | Observed Behavior |
|---|---|
| Within 2 hours of dosing | Convulsion score of 4 |
| After 24 hours of dosing | Convulsion score of 2 or higher |
| After 24 hours of dosing | Motor score of 3 or 4 |
| After 24 hours of dosing | Activity score of 1 |

### Endpoint Sampling

### Scheduled Euthanasia and Sample Collection

Two weeks after dosing, animals were deeply anesthetized via isoflurane. Next, blood was collected, and animals were transcardially perfused with PBS followed by tissue collection.

### Early Euthanasia and Sample Collection

In the event that an animal needed to be euthanized before the scheduled euthanasia timepoint, tissues were collected. No tissues were collected from animals that were found dead (FD).

### Statistical Analysis and Data Graphical Representation

Data was plotted in Prism 9 for Windows (GraphPad Software, Inc.). A mixed-effects analysis was used to evaluate the body weights between groups across time. Tukey's post-hoc test was applied for individual group comparisons. Statistical significance was set at p<0.05.

### In Vivo Results

Three ASOs (Compound_22, Compound_32, and Compound_21) did not go into solution during formulation. Thus, these three treatment groups were not dosed as planned. Six other ASOs (Tominersen, Compound_23, Compound_25, Compound_26, Compound_12, and Compound_89) were only soluble at 80 mg/ml. Thus, animals from these six treatment groups were dosed at 400 μg ASO per mouse instead of the planned 500 μg ASO per mouse. All other ASO dosing went as planned.

Table 10 shows the number of animals in each treatment group that had reached the criteria for early euthanasia (early takedown, or eTD) or were found dead (FD) post-dose.

Table 11 depicts the behavioral observations at baseline, 1 hour, 2 hours, 4 hours, 24 hours, and 1 week post-dose. Abbreviations for behavioral scoring criteria can be found in Table 8. Asterisked items indicate that the animal was found dead after 1 week.

TABLE 10
Number of Animals Deceased or Requiring
Early Takedown in Each Treatment Group

| Treatment | | | # Animals FD per | # eTD per |
|---|---|---|---|---|
| Group | Treatment | N | Treatment Group | Treatment Group |
| 1 | D-PBS | 8 | 0 | 0 |
| 2 | Tominersen | 5 | 0 | 0 |
| 3 | Compound_23 | 6 | 1 | 0 |
| 5 | Compound_25 | 6 | 0 | 0 |
| 6 | Compound_26 | 6 | 0 | 0 |
| 7 | Compound_27 | 6 | 1 | 0 |
| 8 | Compound_12 | 5 | 0 | 0 |
| 9 | Compound_89 | 6 | 0 | 0 |
| 10 | Compound_9 | 6 | 1 | 1 |
| 11 | Compound_11 | 6 | 0 | 1 |
| 12 | Compound_5 | 6 | 0 | 1 |
| 13 | Compound_31 | 6 | 0 | 0 |
| 15 | Compound_2 | 6 | 2 | 0 |
| 16 | Compound_14 | 6 | 0 | 0 |
| 17 | Compound_90 | 5 | 0 | 1 |
| 18 | Compound_91 | 6 | 2 | 0 |
| 19 | Compound_34 | 6 | 0 | 0 |
| 20 | Compound_92 | 6 | 0 | 0 |
| 21 | Compound_18 | 6 | 0 | 0 |
| 22 | Compound_20 | 6 | 0 | 0 |
| 23 | Compound_15 | 6 | 1 | 0 |
| 24 | Compound_4 | 6 | 1 | 0 |
| 25 | Compound_17 | 6 | 0 | 3 |

TABLE 11
Animal Behavioral Scores After Treatment

| | | | Baseline Score | | | | |
|---|---|---|---|---|---|---|---|
| Animal | Treatment | | T = 1 hr | T = 2 hr | T = 4 hr | T = 24 hr | T = 1 wk |
| ID | Group | Treatment | Score | Score | Score | Score | Score |
| 1 | 1 | D-PBS | | | | | |
| 8 | 1 | D-PBS | | | | | |
| 40 | 1 | D-PBS | | | H, P | | |
| 49 | 1 | D-PBS | | | | | |
| 90 | 1 | D-PBS | | | | | |
| 96 | 1 | D-PBS | P | | | | |
| 126 | 1 | D-PBS | | | | B2 | |
| 131 | 1 | D-PBS | | | | B2 | |
| 97 | 2 | Tominersen | H | A5 | A5 | | |
| 102 | 2 | Tominersen | H, P | A5 | A5 | | |
| 107 | 2 | Tominersen | A4, M1, H, | A5, H | A5 | | |
| | | | P | | | | |
| 117 | 2 | Tominersen | | A5 | A5 | | |
| 123 | 2 | Tominersen | H | A5 | A5 | | |
| 41 | 3 | Compound_23 | B2, H, P | | | | |
| 50 | 3 | Compound_23 | B2, P | | | | |
| 57 | 3 | Compound_23 | A3, R1, C2, | A3, M1 | A3, M1, H | H, P | |
| | | | M3, B1, H | | | | |
| 64 | 3 | Compound_23 | A4, B2, P | | | | |
| 70* | 3 | Compound_23 | A3, R1, C1, | A2, R1, M4, | A2, M1, H, | | |
| | | | M2, B2, H | H, P | E | | |
| 76 | 3 | Compound_23 | A3, R1, M2, | A3, R1, M1, | M1, E | | |
| | | | B2 | H | | | |
| 92 | 5 | Compound_25 | A1, R1, H | A1, R1, H | A2, R1, M4 | | |
| 98 | 5 | Compound_25 | A1, R1, B2, | A2, R1, M1, | A2, M1 | | |
| | | | P | B2 | | | |
| 103 | 5 | Compound_25 | A1, R1, C2, | A2, R1, M4 | A2, M2 | | |
| | | | H, P | | | | |
| 108 | 5 | Compound_25 | A2, R1, M3, | A2, R1, M4, | A4, R1, M1 | | |
| | | | B2, H | H, P | | | |
| 124 | 5 | Compound_25 | A2, R1, M1, | A2, R1 | A3, R1, M1 | | |
| | | | B2, H | | | | |
| 125 | 5 | Compound_25 | A1, R1, B2 | A2, R1, M4, | A3, M1 | | |
| | | | | B2 | | | |
| 93 | 6 | Compound_26 | A1, R1, B1, | A2, R1, B2 | A4, M2 | | |
| | | | H | | | | |
| 99 | 6 | Compound_26 | A1, R1, B2 | A1, R1, B1 | A1, R1, B1 | | |
| 104 | 6 | Compound_26 | A1, R1, H | A1, R1, B1 | A2, M2 | | |
| 109 | 6 | Compound_26 | A1, R1, B2, | A1, R1, B1, | A2, R1, M4 | | |
| | | | P | P | | | |
| 114 | 6 | Compound_26 | A1, R1 | A1, B1 | A1, R1 | | |
| 119 | 6 | Compound_26 | A1, R1 | A1 | A1, R1 | | |
| 94 | 7 | Compound_27 | A1, R1, B1, | A1, R1, B1 | A1, R1, B2 | | |
| | | | H | | | | |
| 100 | 7 | Compound_27 | A1, R1, H | A1, R1, B2 | FD | | |
| 105 | 7 | Compound_27 | A1, R1, B1 | A1, R1, B1 | A1, B2 | | |
| 115 | 7 | Compound_27 | A1, R1, B1 | A1, R1, B2 | A1, B1 | | |
| 120 | 7 | Compound_27 | A2, R1, M1, | A1 | A2, R1, M4 | | |
| | | | H | | | | |
| 122 | 7 | Compound_27 | A1, R1, B2 | A1 | A1, R1 | | |
| 95 | 8 | Compound_12 | A1, R1, P | A2, R1, H | A3, R1, M2 | | |
| 101 | 8 | Compound_12 | A1, R1, B1, | A1, R1 | A3, M3 | | |
| | | | P | | | | |
| 106 | 8 | Compound_12 | A1, R1, P, | A1, R1, B1 | A3, M3 | | |
| | | | E | | | | |
| 116 | 8 | Compound_12 | A3, R1, B1, | A2, R1, M4 | A3, M2 | | |
| | | | H, P | | | | |
| 121 | 8 | Compound_12 | A2, R1, H | A2, R1, M4 | A3, R1, M1 | | |
| 42 | 9 | Compound_89 | A1, R1, E | A1, R1 | A1, R1, H, | | |
| | | | | | E | | |
| 43 | 9 | Compound_89 | A2, R1, M3, | A1, R1, H, | A3, M1, H, | P | |
| | | | H | E | P | | |
| 52 | 9 | Compound_89 | A1, R1, C3, | A1, R1, H | A2, R1, M3, | H | |
| | | | H | | H | | |
| 65 | 9 | Compound_89 | A1, R1, H | A1, R1, H | A2, R1, M3, | | |
| | | | | | H, E | | |
| 71 | 9 | Compound_89 | A1, R1, H, | A1, R1, B1, | A1, H, E | P | |
| | | | P | E | | | |
| 77 | 9 | Compound_89 | A1, R1, B2, | A1, R1, B1, | A2, R1, M2, | | |
| | | | H | H | B1, H | | |
| 47 | 10 | Compound_9 | A1, R1, C3, | A1, R1, B1, | A1, R1, B1, | H | |
| | | | B1 | H | H | | |
| 51 | 10 | Compound_9 | A1, R1, B1 | A1, B1 | A1, R1, B1, | P, H | |
| | | | | | H, E | | |
| 54 | 10 | Compound_9 | A1, R1, C3, | A1, R1, B1, | A1, R1, B1, | | |
| | | | H | H, P, E | H | | |
| 59 | 10 | Compound_9 | FD | | | | |
| 66 | 10 | Compound_9 | C4, eTD | | | | |
| 78 | 10 | Compound_9 | A1, R1, C3, | A1, R1, B1, | A1, R1, B1, | A5, R2 | |
| | | | B1, H | H | H, E | | |
| 82 | 11 | Compound_11 | A1, R1, H | A2, R1, M3, | A1, R1, H, | | |
| | | | | H | E | | |
| 84 | 11 | Compound_11 | A2, R1, B2, | A1, R1, B1, | A2, M2, H | | |
| | | | H | H, P | | | |
| 85 | 11 | Compound_11 | A1, R1, B1, | A1, R1, B1, | A2, R1, M3, | | |
| | | | H | H, P | H, E | | |
| 86 | 11 | Compound_11 | C4, eTD | | | | |
| 87 | 11 | Compound_11 | A1, R1, B1, | A1, R1, B1, | A1, B2, H, | P | |
| | | | H | H, E | P, E | | |
| 88 | 11 | Compound_11 | n/a | A1, R1, H, | A1, R1, H, | P | |
| | | | | E | E | | |
| 2 | 12 | Compound_5 | C4, eTD | | | | |
| 9 | 12 | Compound_5 | A1, R1, C3, | A1, R1, B1 | A1 | | |
| | | | B1 | | | | |
| 15 | 12 | Compound_5 | A1, C3, B1 | A1, B1 | A2 | | |
| 21 | 12 | Compound_5 | A1, R1, C3 | A1, R1 | A1, R1, B2 | | |
| 27 | 12 | Compound_5 | A2, R1, C1, | A2, R1, M4 | A1, R1 | | |
| | | | C3, M3 | | | | |
| 33 | 12 | Compound_5 | A2, M4 | A1, B1 | A3, M3 | | |
| 127 | 13 | Compound_31 | A3, R1, M2 | M2, H | M1, H | | |
| 132 | 13 | Compound_31 | A4, M1, H, | M2 | M1 | B2 | A5 |
| | | | P | | | | |
| 136 | 13 | Compound_31 | A4, M1, H, | A4, M1, H | M1, H | | |
| | | | L | | | | |
| 140 | 13 | Compound_31 | A4, M1 | A4, M2 | M1 | P, R2 | |
| 144 | 13 | Compound_31 | A4, M2, P | A4, M2, P | A4, M1 | | |
| 148 | 13 | Compound_31 | A3, M1, H, | A4, M1, P | A4, P | | |
| | | | P | | | | |
| 3 | 15 | Compound_2 | A2, M1, B2 | M1 | | | |
| 10 | 15 | Compound_2 | FD | | | | |
| 16 | 15 | Compound_2 | A4 | A3, M1 | M1 | | |
| 22 | 15 | Compound_2 | A4, M1 | | | | R1, M1, HT, |
| | | | | | | | P |
| 28 | 15 | Compound_2 | A4, R1, M2, | | | | |
| | | | B2 | | | | |
| 34 | 15 | Compound_2 | A4, M1 | M1 | | | FD |
| 128 | 16 | Compound_14 | A4, R1, M1, | M1, H, P | H | | |
| | | | H | | | | |
| 133 | 16 | Compound_14 | A4, M1, H, | A4, M2, P | A4, M1, P | | |
| | | | P | | | | |
| 137 | 16 | Compound_14 | A4, M1, H | A4, M2, H | A4, M1, P | | |
| 141 | 16 | Compound_14 | A4, M2 | A4, M2 | A3, H | | |
| 145 | 16 | Compound_14 | A4, M1 | M1 | M1 | | |
| 149 | 16 | Compound_14 | A4, M1, H, | M1, P | A4, P | | |
| | | | P | | | | |
| 44 | 17 | Compound_90 | A2, R1, M4, | A1, R1, H | A3, M2, H | | |
| | | | H, P | | | | |
| 60 | 17 | Compound_90 | C4, eTD | | | H, P | |
| 67 | 17 | Compound_90 | A1, R1, B1, | A1, R1, H | A2, R1, M4, | | |
| | | | H | | H, E | | |
| 73 | 17 | Compound_90 | A2, R1, M3, | A1, R1, C1, | A2, M2, H | | |
| | | | B1, H | B1, H | | | |
| 79 | 17 | Compound_90 | A1, H, P | A1, R1, H, | M1, H | | |
| | | | | P | | | |
| 4 | 18 | Compound_91 | A1, R1, B1 | A1, R1, B1 | A1, R1, B1 | H, P | |
| 11 | 18 | Compound_91 | A1, R1, B1 | A1, R1, B1 | A1, R1 | R1 | |
| 17 | 18 | Compound_91 | A1, C3, B1 | A1, B1 | A1 | | |
| 23 | 18 | Compound_91 | A2, R1, M4 | A1, R1, B1 | A1, R1 | | |
| 29 | 18 | Compound_91 | A1, R1 | A1, R1, B1 | A1, R1 | | |
| 35 | 18 | Compound_91 | A1, R1, C3, | A1, R1, B1 | A1, R1, B1 | | |
| | | | B1 | | | | |
| 45 | 19 | Compound_34 | A1, R1, B1, | A1, R1, B1, | A1, R1, B2, | | |
| | | | H | H | H | | |
| 56 | 19 | Compound_34 | A1, R1, C3, | A1, R1, H | A1, R1, B1, | H, M1 | |
| | | | B1, H, P | | H, E | | |
| 63 | 19 | Compound_34 | A1, R1, B1, | A1, R1, H | A3, M2, H, | | |
| | | | H | | E | | |
| 68 | 19 | Compound_34 | A1, R1, B2, | A1, R1, H | A1, R1, H, | P, H | |
| | | | H | | E | | |
| 74 | 19 | Compound_34 | A1, R1, B1, | A1, R1, B1, | A1, R1, B1, | | |
| | | | H | H | H, E | | |
| 80 | 19 | Compound_34 | A1, R1, B2 | A1, R1, B1, | A1, R1, B1, | P | |
| | | | | H | H, E | | |
| 5 | 20 | Compound_92 | P | | | | |
| 12 | 20 | Compound_92 | B2, P | | | | |
| 18 | 20 | Compound_92 | | | | | |
| 24 | 20 | Compound_92 | | | | | |
| 30 | 20 | Compound_92 | M3 | | B2 | | |
| 36 | 20 | Compound_92 | | | | | |
| 48 | 21 | Compound_18 | A2, R1, M4, | A2, M3, B2, | A2, R1, B1, | P | |
| | | | H, P | H | P, E | | |
| 55 | 21 | Compound_18 | A1, R1, B1, | A1, H | A1, R1, B1, | P | |
| | | | H, P | | H, E | | |
| 62 | 21 | Compound_18 | A1, R1, H | A1, R1, H | A1, R1, B2, | | |
| | | | | | H, P | | |
| 69 | 21 | Compound_18 | A2, R1, M4, | A1, R1, H | A1, R1, H, | | |
| | | | B1 | | E | | |
| 75 | 21 | Compound_18 | A1, R1, H | A1, R1, B1, | A1, R1, B1, | | |
| | | | | H | H, E | | |
| 81 | 21 | Compound_18 | A1, R1, B2, | A1, R1, B1, | A1, H, E | P | |
| | | | H, P | H, P | | | |
| 129 | 22 | Compound_20 | A4, M1, B2, | P | | B2 | |
| | | | H | | | | |
| 134 | 22 | Compound_20 | A4, M1, P | | | | |
| 142 | 22 | Compound_20 | A4, P | | | | A5 |
| 146 | 22 | Compound_20 | A4, M1 | A4, M1 | A4, H, P | | |
| 150 | 22 | Compound_20 | B2, H, E | H | | P | |
| 155 | 22 | Compound_20 | H, P | | A5, R2 | R2, B2 | A5 |
| 6* | 23 | Compound_15 | A4, R1, C2 | | | | |
| 13 | 23 | Compound_15 | | | | | |
| 19 | 23 | Compound_15 | M3 | | | | |
| 25 | 23 | Compound_15 | | | | H | |
| 31 | 23 | Compound_15 | C3 | | | | |
| 37 | 23 | Compound_15 | | | | | |
| 7 | 24 | Compound_4 | A1, R1, B1 | A1, R1, B1 | A1, R1 | | |
| 14 | 24 | Compound_4 | A1, R1, B1 | A1, R1, B1 | A1, R1 | | |
| 20* | 24 | Compound_4 | A1, R1 | A1, R1 | A3, M1 | | |
| 26 | 24 | Compound_4 | A1, R1, C3 | A1, R1, B1 | A1, R1, B1 | | |
| 32 | 24 | Compound_4 | C3 | A4, R1, M4 | A1, R1 | | |
| 39 | 24 | Compound_4 | A2, R1, M3 | A2, R1, M3 | A3, M2 | | |
| 130 | 26 | Compound_17 | A4, M1, H | M1, P | M1, H | | |
| 135 | 26 | Compound_17 | A4, M1, B2, | A4, M2, P | M1, H, P | | |
| | | | H | | | | |
| 139 | 26 | Compound_17 | A4, M1, B2, | A5, M1 | | | |
| | | | H | | | | |
| 143 | 26 | Compound_17 | A4, M1 | M1 | | | A4, HT |
| 147 | 26 | Compound_17 | A4 | A4, M1 | M3, H, P | | |
| 151 | 26 | Compound_17 | H | H | P | | A4, R1, HT |

### Example 3: UBE3A Knockdown by ASOs at mRNA and Protein Levels in F-Dup Neurons

This example illustrates experiments that were conducted to test the effect of certain exemplary ASOs of the present disclosure on UBE3A mRNA level and protein expression levels in neuronal cells derived from human induced pluripotent stem cells (iPSCs), which were acquired by dedifferentiating cells from Dup15q patients (F-Dup Neurons).

UBE3A knockdown was assessed at mRNA and protein levels after treatment of the neuronal cells with ten exemplary ASOs of the present disclosure. Human induced pluripotent stem cell-derived (hiPSC-derived) F-Dup neurons and corrected neurons (neuronal cells derived from human iPSCs, which were sourced from Dup 15q patient but had been genetically “corrected” to remove the supernumerary chromosome) were seeded at 150,000 cells/well in 96-well plates. POS1 and POS2 targeting UBE3A were used as positive controls for qPCR. Scr GFP_ASO, scr P1, and NEAT1 ASO were used as negative controls in F-Dup and corrected, untreated cells. scr Pq was used as the negative control for qPCR only.

ASOs were delivered by gymnosis. Control ASOs were used at a concentration of 5 μM for qPCR. Test and control ASOs were delivered at concentrations around 6.33 μM and 20 μM. Each ASO plate source was tested in duplicate on the same cell plate, and lysate from each cell well was tested in technical duplicates on the same qPCR plate, in addition to technical singlicates in separate runs.

hiPSC-derived F-Dup neurons and corrected neurons were cultured for a total of about 21 days, and culture medium was refreshed every two or three days. Cells were treated with ASOs starting on the 7^{th} day of culturing for seven days, after which the cells were cultured in culture medium without ASO supplementation for an additional seven days, including the day of transition from ASO supplementation to no ASO supplementation. These ASO-treated cells were then harvested on the 14^{th} day of culturing, or on the 7^{th} day after the first ASO treatment, and subsequently harvested every two or three days thereafter for a total of three lysate harvests.

Specifically, cell lysate was harvested for qPCR and protein analysis on the 7^{th} 10^{th}, and 14^{th} days after the first day of ASO treatment, or equivalent to the 14^{th} 17^{th} and 21^{st} days of total culture time. The wash-out phase of culturing began after termination of ASO treatment and the transition to using non-ASO culture medium and lasted 7 days.

### Cell Lysate Preparation

Lysis was done with RIPA Lysis and Extraction Buffer (Thermo Fisher catalog #89900) supplemented with Halt Protease Inhibitor Cocktail (Thermo Fisher catalog #1861278) at 30 μl buffer per condition per well. Lysis preparation for qPCR analysis included using a lysis solution supplemented with DNase I (25 μl/well) and stop solution (2.5 μl/well) from the Invitrogen Cells-to-CT 1-Step TaqMan Kit (Thermo Fisher catalog #A25602).

### Protein Quantification

Total protein level of the cell lysates was quantified by assessing protein yield with a Bicinchoninic acid (BCA) assay, which provides a colorimetric detection and quantitation of total protein. Two biological replicates were evaluated for each test ASO, positive and negative control ASO, and untreated cells. UBE3A protein level was quantified by Jess Western blot. The primary antibody used was Proteintech's UBE3A antibody from a rabbit host (Proteintech catalog #10344-1-AP) and diluted 1:10 in 10% goat serum. The secondary antibody used was an anti-rabbit secondary HRP antibody, 1λ. Each sample was normalized to a final concentration of total protein.

Sample Preparation for qPCR Analysis

15% of the volume of prepared lysate from each sample was used in a qPCR reaction with TaqMan Master Mix from the Invitrogen Cells-to-CT 1-Step TaqMan Kit. A FAM-labeled human UBE3A probe was used concurrently with the Applied Biosystems™ Human RPLP0 (large ribosomal protein) Endogenous Control (VIC™/TAMRA Probe, Primer Limited).

### Experimental Setup

Ten ASOs were tested in duplicate at two concentrations (6.3 μM and 20 μM) in each cell plate. Seven cell plates were used. ASO controls were included in one concentration (5 μM) in every plate for normalization of qPCR data for each plate during data analysis. NEAT1 ASO, F-Dup Non-treated and Corrected Non-treated were used as reference points to assess ASO-induced knockdown. All test ASOs at their two concentrations and one set of controls (POS1, POS2, NEAT1 ASO, scr GFP_ASO, scr P1, F-Dup Non-treated, and Corrected Non-treated) were processed in Bio-Techne's ProteinSimple Jess automated Western Blot instrument for protein quantification by protein separation and immunodetection.

Assay Performance of Control and Test ASOs Across qPCR Plates at Treatment Days 7 and 10 (mRNA)

The UBE3A assay performed consistently with high UBE3A knockdown levels and limited variability in the control ASO populations. Successful ASO delivery was confirmed by >65% and >40% UBE2A knockdown by POS1 and POS2, respectively, versus NEAT1 ASO, which did not result in UBE3A mRNA knockdown. Positive control ASOs had similar levels of UBE3A mRNA knockdown on both treatment days 7 and 10 and thus indicate that there is a clear and stable effect.

UBE3A Knockdown at mRNA and Protein Levels Normalized to NEAT1 ASO at ASO Treatment Days 7, 10, and 14

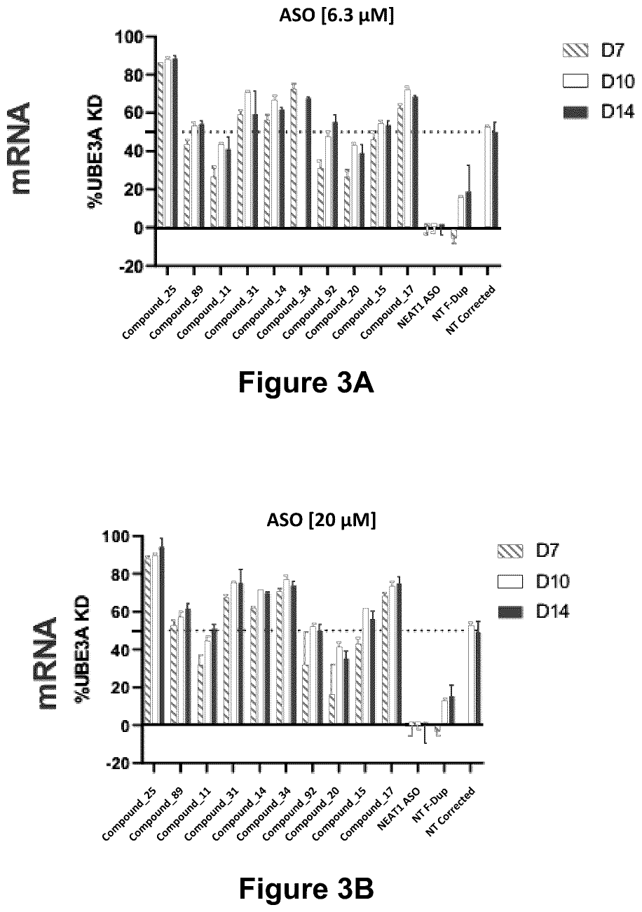

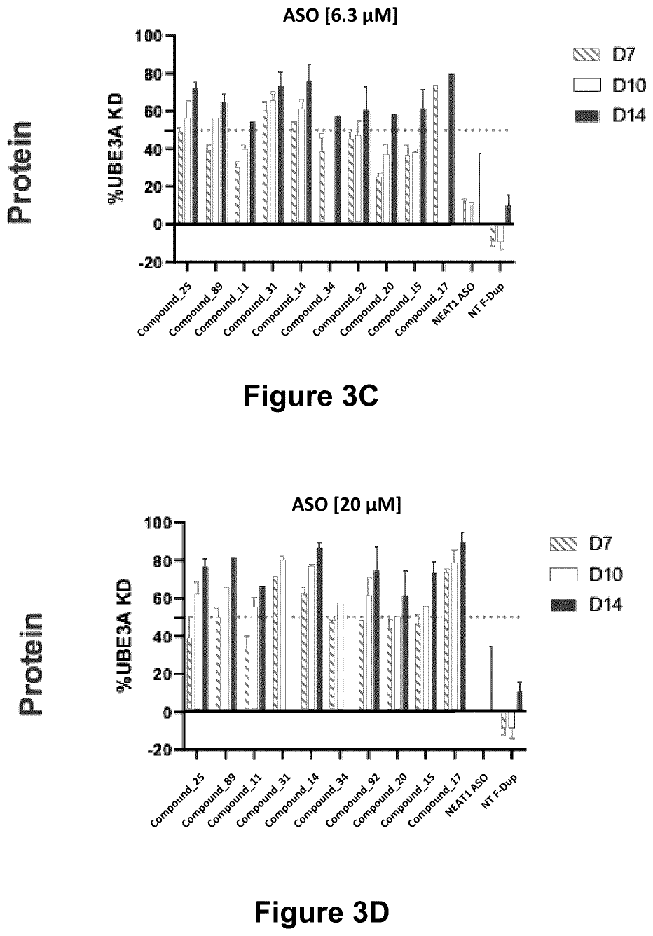

ASO treatment via gymnosis with UBE3A-targeting ASOs resulted in concentration-dependent UBE3A protein knockdown for test ASOs that were normalized to NEAT1 ASO on ASO treatment days 7,10, and 14, equivalent to wash-out days 0, 3, and 7, or the 14^{th} 17^{th} and 21^{st} days of time in culture (FIGS. 3C-3D). The UBE3A mRNA knockdown effect was only slightly increased at day 10, suggesting that there may have been abundant intracellular ASO present on day 7 at these concentrations (6.3 μM and 20 μM) (FIGS. 3A-3B). Compound_25 resulted in the highest UBE3A mRNA knockdown, while Compound_11 and Compound_20 resulted in the lowest UBE3A mRNA knockdown.

All test ASOs induced concentration- and time-dependent UBE3A protein knockdown, corroborating the effect observed at the mRNA level. Data presented in the figures of this example represent the average of technical singlicates +/−SD per condition (biological replicates n=2), corresponding to each treatment day.

UBE3A protein knockdown increased progressively with longer ASO treatments. By treatment day 10, all 10 exemplary test ASOs were able to knock down 50-80% of UBE3A protein at 20 μM. By treatment day 14, all exemplary test ASOs induced >60% UBE3A protein knockdown at 20 μM.

No datapoints were obtained for Compound_34 (mRNA & protein) and Compound_17 (protein) at 6.3 μM at treatment Day 10. No datapoints were collected for Compound_31 and Compound_34 (protein) at 20 μM at treatment Day 14. No datapoints were collected for NT corrected (mRNA) at 6.3 μM at treatment Day 7.

Comparison of UBE3A mRNA and Protein Expression Levels Between F-Dup and Corrected Neurons

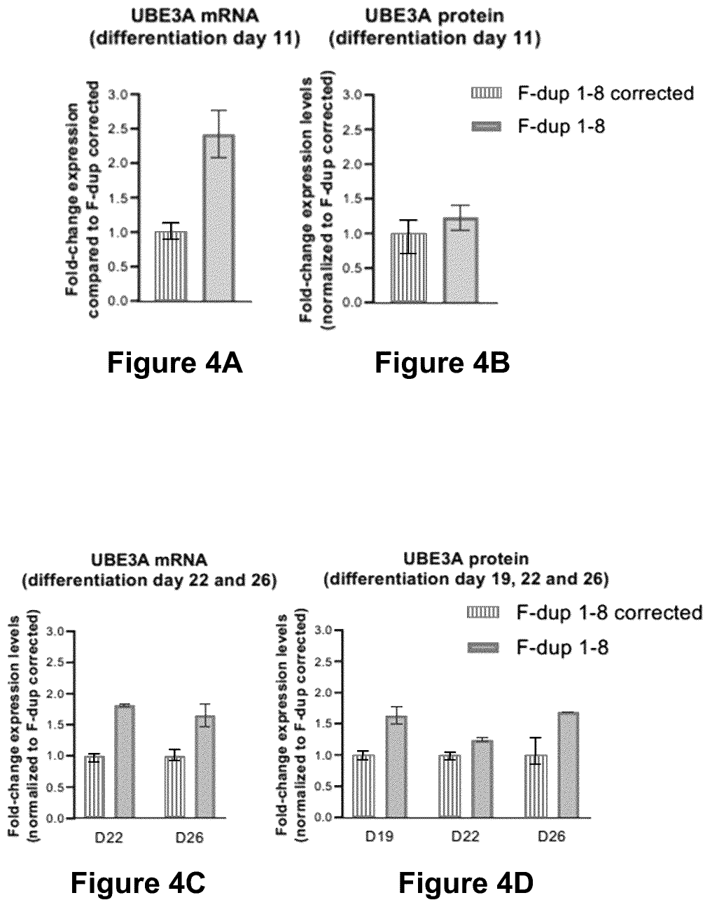

Relative UBE3A mRNA expression level between F-Dup 1-8 cells and F-Dup 1-8 corrected cells on differentiation day 11, equivalent to the day before initiation of ASO treatment, indicate about a 2.5-fold difference (FIG. 4A). Relative UBE3A protein expression level between F-Dup 1-8 cells and F-Dup 1-8 corrected cells on differentiation day 11, equivalent to the day before initiation of ASO treatment, indicate about 1.25-fold difference (FIG. 4B).

On differentiation days 19, 22, and 26, equivalent to the 14^{th} 17^{th} and 21^{st} days of culture, or the 0^{th} 3^{rd}, and 7^{th} days after ASO treatment was terminated and non-ASO culture medium was used, expression of UBE3A mRNA and UBE3A protein were evaluated. UBE3A mRNA was generally elevated in F-Dup 1-8 cells. More specifically, expression of UBE3A mRNA was elevated by about 2-fold in F-Dup 1-8 cells relative to F-Dup 1-8 corrected cells (FIG. 4C); however, there was a smaller fold change between the two cell lines for UBE3A protein expression levels (between about 1.24-fold to about 1.6-fold) (FIG. 4D). This difference in relative mRNA vs protein expression may be explained by different regulation pathways of the additional copies of UBE3A, or a short protein half-life that prevents excessive accumulation in the cells.

Data presented in FIGS. 4A-4D represent the average of technical duplicates +/−SD per condition (biological replicates n=2-4), corresponding to each differentiation day. No data was collected for mRNA from the Corrected neurons on differentiation Day 19.

### UBE3A Protein Expression Levels at ASO Treatment Days 7 and 10

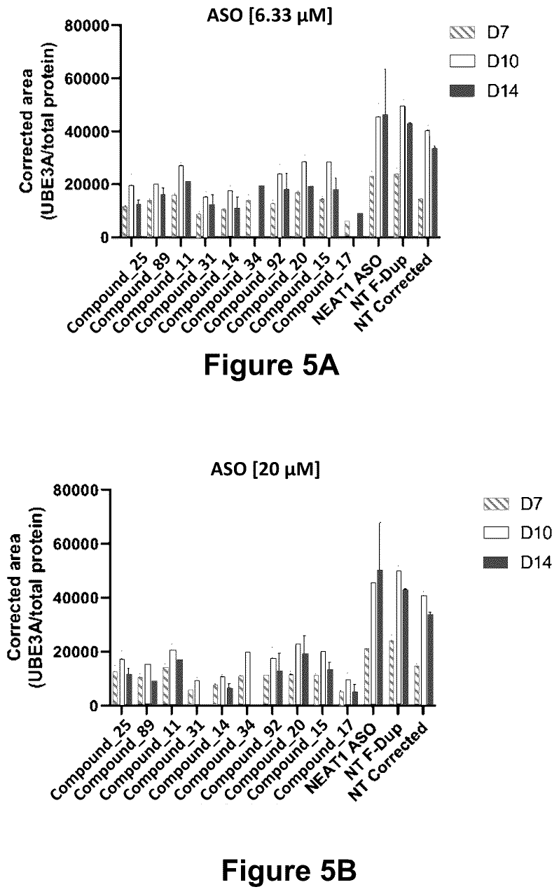

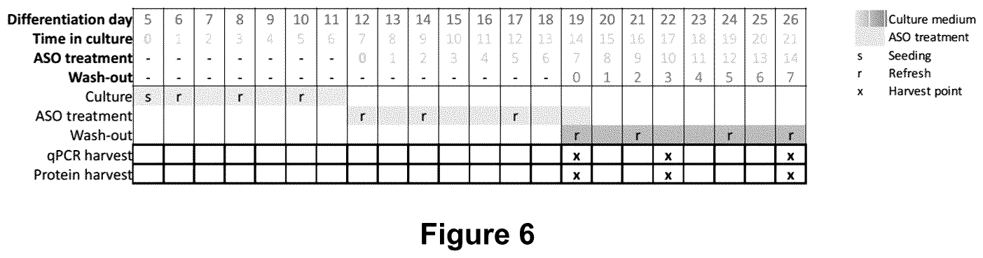

ASO treatment via gymnosis with UBE3A-targeting ASOs resulted in concentration-responsive and time-dependent UBE3A protein knockdown for exemplary test ASOs that were normalized to NEAT1 ASO (FIGS. 5A-5B). Protein expression levels were normalized to total protein output. Data represent the average of technical singlicates +/−SD per condition (biological replicates n=2), corresponding to each treatment day (NT=non-treated). No data points were collected for 6.3 μM for Compound_34 on treatment Day 10, or for Compound_17 on treatment Days 10 and 14. No datapoints were collected for 20 μM Compound_31 or Compound_34 on treatment Day 14. D7, D10, and D14 on the figures represent day 7, day 10, and day 14 after ASO treatment (treatment Days 7, 10, 14), respectively.

TABLE 12
Summary of Results for 10 Exemplary ASOs in Rat and iPSC-derived Neuron Studies

| | Primary | Primary screen | | | | | | ASO validation in rat fibroblasts | | | | | |
|---|---|---|---|---|---|---|---|---|---|---|---|---|---|
| | screen/Potency | (ASO 100 nM, Hs) | | | | Summary | | (ASO 100 nM top concentration, Rn) | | | | | |
| | determination/Rat | AVE | SD | UBE3A | SD | UBE3A | | AVE | SD | UBE3A | SD | | Immunotoxicity |
| | validation/ | cell | cell | KD | UBE3A | AVE | EC50 | cell | cell | KD | UBE3A | Sigmoidal | High/medium/ |
| Compound ID | Immunotox | loss | loss | (%) | KD | KD | (nM) | loss | loss | (%) | KD | CRC | low |
| Compound 25 | 3/8/8/9 | 10.5 | 6.2 | 40.1 | 0.8 | 36.9 | 0.9 | 7 | 2.8 | 84.3 | 2.1 | Yes | Low |
| Compound_89 | 3/8/8/9 | 5.2 | 1.2 | 70.0 | 3.7 | 68.6 | 2.5 | 4.4 | N.A. | 68.5 | 3.7 | Yes | Med |
| Compound_11 | 3/4/6/5 | 14.2 | 3.5 | 79.7 | 2.2 | 68.9 | 1.4 | −6.8 | 5.8 | 26.9 | 13.1 | Yes | Low |
| Compound_31 | 3/4/6/7 | 0.2 | 2.7 | 76.1 | 2.2 | 52.8 | 0 | −30.5 | 3.1 | 63.4 | 1.5 | Yes | Low |
| Compound_14 | 3/4/6/7 | 1.4 | 7.4 | 77.6 | 1 | 69.3 | n.d. | −10.5 | 9.3 | 58.4 | 13.7 | Yes | Low |
| Cmpound_34 | 3/8/8/9 | 2.2 | 0.8 | 49.6 | 1.2 | 48.7 | 1.7 | −1.3 | 7.3 | 76.1 | 5.1 | Yes | Low |
| Compound_92 | 3/8/8/9 | −7.7 | 2.1 | 62.9 | 4.8 | 58.5 | 1.1 | −11 | 3.8 | 43.7 | 5.7 | Yes | Low |
| Compound_20 | 3/4/6/7 | 13.6 | 3.2 | 62.8 | 1.6 | 57.2 | n.d. | −10.3 | 2.4 | 49.3 | 1.1 | Yes | Low |
| Compound_15 | 3/4/6/5 | 3.5 | 2.8 | 77.4 | 1.3 | 72.7 | 3.2 | −5.1 | 1.2 | 57.7 | 3.8 | Yes | Low |
| Compound_17 | 3/4/6/7 | −23.6 | 1.8 | 75.8 | 1.9 | 54.4 | 0.1 | −21.9 | 4.0 | 53.9 | 2.4 | Yes | Low |

TABLE 13
Summary of UBE3A Percentage of Knockdown Results for RNA and Protein

| | | | | Exp16_ | Exp16_ | | Exp16_ | | Expl6_s | |
|---|---|---|---|---|---|---|---|---|---|---|
| | | | | F-Dup | F-Dup | | F-Dup | | F-Dup | |
| | | | | neurons | neurons | | neurons | | neuron | |
| | | | | Ph3A | Ph3B | | Ph3B | | Ph3B | |
| Compound | | Sequence | | (D7) | (D7) | | (D10)\| | | (D14) | |
| ID | Length | (1) | | RNA | RNA | Protein | RNA | Protein | RNA | Protein |
| Compound_25 | 20 | AGCTGTGGCC | | 93 | 86.9 | 39.1 | 89.8 | 61.9 | 94.2 | 78.2 |
| | | ATTCGGTGAC | | | | | | | | |
| Compound_89 | 19 | TTTGTTGCAA | SoW2 | 62.6 | 52.8 | 48.7 | 56.5 | 65.9 | 61.5 | 81.4 |
| | | TAGGCTTGA | | | | | | | | |
| Compound_11 | 19 | CTTCTGGTCT | | 53.3 | 31.8 | 32.4 | 43.9 | 54.9 | 50.9 | 65.7 |
| | | GAATAAGTA | | | | | | | | |
| Compound_31 | 18 | CATTCTCCGA | | 85 | 67.3 | 71.5 | 75.3 | 79.7 | 75.2 | N/A |
| | | ATCTGGTC | | | | | | | | |
| Compound_14 | 20 | TCCATAGCGA | | 87.8 | 61.2 | 62.3 | 71 | 76 | 69.7 | 87.1 |
| | | TCATCTCTAG | | | | | | | | |
| Compound_34 | 18 | AAGCTGTGG | | 87.9 | 70.6 | 46.7 | 76.9 | 56.8 | 73.8 | N/A |
| | | CCATTCGGT | | | | | | | | |
| Compound_92 | 20 | TTCCCAGAAC | | 82.8 | 31.6 | 48 | 52.1 | 61.3 | 49.8 | 74.9 |
| | | TCCCTAATCA | | | | | | | | |
| Compound_20 | 20 | TAGGTAACCT | | 42.2 | 15.9 | 43.6 | 41.2 | 50.2 | 35 | 61.4 |
| | | TTCTGTGTCT | | | | | | | | |
| Compound_15 | 20 | TTCCGGCTTC | | 71.4 | 42.9 | 46.3 | 60.7 | 56.1 | 56. | 74.4 |
| | | CACATATAAG | | | | | | | | |
| Compound_17 | 20 | CCTTCCTGTT | | 78.8 | 68.5 | 73.8 | 73.5 | 78.9 | 74.6 | 90 |
| | | TTCATTTGTA | | | | | | | | |

TABLE 14
Assay Performance: Acceptance Criteria and Results for JESS Runs (Protein)
ASO [20 μM]

| | Day 7 | | | Day 10 | | | Day 14 | | |
|---|---|---|---|---|---|---|---|---|---|
| | Corrected | % | | Corrected | % | | Corrected | % | |
| Compound | Area | UBE3A | | Area | UBE3A | | Area | UBE3A | |
| ID | UBE3A | KD | S/N >20 | UBE3A | KD | S/N >20 | UBE3A | KD | S/N >20 |
| Compound_25 | 11809.6 | 49 | All samples passed | 19890.8 | 56.6 | All samples passed | 11670.35 | 72.5 | All samples passed |
| Compound_89 | 14140.6 | 39 | All samples passed | 20005.5 | 56.3 | 1 sample is <20, | 16312.35 | 64.5 | All samples passed |
| | | | | | | Excluded | | | |
| Compound_11 | 16266.05 | 29.8 | All samples passed | 27441.9 | 40.1 | All samples passed | 21073 | 54.5 | All samples passed |
| Compound 31 | 9088.65 | 60.8 | All samples passed | 15652.45 | 65.8 | All samples passed | 12244.55 | 73.3 | All samples passed |
| Compound_14 | 10782.55 | 53.5 | All samples passed | 17771.3 | 61.2 | All samples passed | 10684.2 | 76.1 | All samples passed |
| Compound_34 | 14238.2 | 38.5 | All samples passed | N/A | N/A | 1 sample is <20, | 19534.9 | 57.8 | 1 sample is <20, |
| | | | | | | Excluded | | | Excluded |
| Compound_92 | 12990.85 | 43.9 | All samples passed | 24263.2 | 47 | All samples passed | 18380.45 | 60.4 | All samples passed |
| Compound_20 | 17315.05 | 25.3 | All samples passed | 28864.9 | 37 | All samples passed | 19336.8 | 58.2 | 1 sample is <20, |
| | | | | | | | | | Excluded |
| Compound_15 | 14725.3 | 36.5 | All samples passed | 28204.75 | 38.4 | All samples passed | 15995.3 | 61.4 | All samples passed |
| Compound_17 | 6222.6 | 73.1 | 1 sample is <20, | N/A | N/A | 1 sample is <20, | 9244.4 | 80.1 | 1 sample is <20, |
| | | | Excluded | | | Excluded | | | Excluded |
| NEAT1 ASO | 23162.3 | 0 | All samples passed | 45765.9 | 0 | All samples passed | 45328.7 | 0 | All samples passed |
| NT F-Dup | 24158.85 | •9.3 | All samples passed | 49675.35 | −8.6 | All samples passed | 43114.25 | 10.5 | All samples passed |
| NT Corrected | 14823.7 | N/A | All samples passed | 40691.2 | N/A | All samples passed | 25586.9 | N/A | All samples passed |

TABLE 15
Assay Performance: Acceptance Criteria and Results for JESS Runs (Protein)

| | Day 7 | | | Day 10 | | | Day 14 | | |
|---|---|---|---|---|---|---|---|---|---|
| | Corrected | % | | Corrected | % | | Corrected | % | |
| Compound | Area | UBE3A | | Area | UBE3A | | Area | UBE3A | |
| ID | UBE3A | KD | S/N >20 | U8E3A | KD | S/N >20 | U8E3A | KD | S/N >20 |
| Compound_25 | 12797 | 39.1 | All samples passed | 17438.3 | 61.9 | All samples passed | 11674.6 | 76.7 | All samples passed |
| Compound_89 | 10781.15 | 48.7 | All samples passed | 15577.3 | 65.9 | All samples passed | 9228 | 81.6 | All samples passed |
| Compound_11 | 14212.2 | 32.4 | All samples passed | 20611.8 | 54.9 | All samples passed | 16974.7 | 66.2 | All samples passed |
| Compound 31 | 5981.7 | 71.5 | All samples passed | 9279.4 | 79.7 | All samples passed | N/A | N/A | 1 sample is <20, |
| | | | | | | | | | Excluded |
| Compound_14 | 7942.25 | 62.3 | All samples passed | 11001.5 | 76 | All samples passed | 6692.3 | 86.7 | All samples passed |
| Compound_34 | 11198.25 | 46.7 | All samples passed | 19762.1 | 56.8 | All samples passed | N/A | N/A | 1 sample is <20, |
| | | | | | | | | | Excluded |
| Compound_92 | 10940.2 | 48 | All samples passed | 17709.4 | 61.3 | All samples passed | 12849.65 | 74.4 | All samples passed |
| Compound_20 | 11852 95 | 43.6 | All samples passed | 22762.85 | 50.2 | All samples passed | 19284.35 | 61.6 | All samples passed |
| Compound_15 | 11290.85 | 46.3 | All samples passed | 20048.9 | 56.1 | 1 sample at the limit | 13289.4 | 73.5 | All samples passed |
| | | | | | | (20.9), Excluded | | | |
| Compound_17 | 5499.05 | 73.8 | All samples passed | 9662.3 | 78.9 | All samples passed | 5175.45 | 89.7 | All samples passed |
| NEAT1ASO | 21018.4 | 0 | All samples passed | 45711.1 | 0 | 1 sample at the limit | 50200.4 | 0 | All samples passed |
| | | | | | | (21.8), Excluded | | | |
| NT F-Dup | 24158.85 | −9.3 | All samples passed | 49675.35 | −8.6 | All samples passed | 43114.25 | 10.5 | All samples passed |
| NT Corrected | 14823.7 | N/A | All samples passed | 40691.2 | N/A | All samples passed | 33736.6 | N/A | All samples passed |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

1-126. (canceled)

127. A pharmaceutical composition for use in a method of reducing expression of a UBE3A protein in a mammalian cell having duplication, overexpression, or a gain-of-function mutation of a UBE3A gene that encodes the UBE3A protein, the method comprising contacting the pharmaceutical composition to the mammalian cell, wherein the pharmaceutical composition comprises an antisense oligomer or a vector encoding the antisense oligomer, and wherein the antisense oligomer reduces a level of a processed mRNA encoding the UBE3A protein in the mammalian cell.

128. The pharmaceutical composition for use according to claim 127, wherein the antisense oligomer comprises a polynucleotide sequence that is at least 80% complementary to at least 8 contiguous nucleotides of a sequence set forth in any one of SEQ ID NOs: 93-153.

129. The pharmaceutical composition for use according to claim 127, wherein the antisense oligomer comprises a sequence with at least 80%, at least 90%, or 100% identity to a sequence set forth in any one of SEQ ID NOs: 1-92.

130. The pharmaceutical composition for use according to claim 127, wherein the pharmaceutical composition comprises the antisense oligomer, and wherein the antisense oligomer comprises:
(a) a backbone modification, a modified sugar moiety or a combination thereof;
(b) a phosphorothioate linkage or a phosphorodiamidate linkage; and/or
(c) a phosphorodiamidate morpholino, a locked nucleic acid, a peptide nucleic acid, a 2′-O-methyl moiety, a 2′-Fluoro moiety, a 2′-O-methoxyethyl moiety, or a 2′-NMA moiety.

131. The pharmaceutical composition for use according to claim 127, wherein the pharmaceutical composition comprises the antisense oligomer, and wherein the antisense oligomer comprises at least one modified sugar moiety.

132. The pharmaceutical composition for use according to claim 127, wherein the pharmaceutical composition comprises the antisense oligomer, and wherein the antisense oligomer comprises three, four, five, or six 2′-O-methoxyethyl modified nucleosides at a 5′ end of the antisense oligomer; three, four, five, or six 2′-O-methoxyethyl modified nucleosides at a 3′ end of the antisense oligomer; and a phosphorothioate linkage between any two neighboring nucleosides of the antisense oligomer.

133. The pharmaceutical composition for use according to claim 127, wherein the pharmaceutical composition comprises the antisense oligomer, and wherein the antisense oligomer comprises:
a 5′ region consisting of three, four, five, or six linked nucleosides;
a central region consisting of eight, nine, ten, eleven, or twelve linked nucleosides; and
a 3′ region consisting of three, four, five, or six linked nucleosides;
wherein each of the three, four, five, or six linked nucleosides in the 5′ region and each of three, four, five, or six linked nucleosides in the 3′ region comprises a modified sugar moiety, and wherein each of the eight, nine, ten, eleven, or twelve linked nucleosides in the central region is a deoxyribonucleoside.

134. The pharmaceutical composition for use according to claim 127, wherein the antisense oligomer consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 12 to 50 nucleobases, 12 to 40 nucleobases, 12 to 35 nucleobases, 12 to 30 nucleobases, 12 to 25 nucleobases, 12 to 20 nucleobases, 12 to 15 nucleobases, 15 to 50 nucleobases, 15 to 40 nucleobases, 15 to 35 nucleobases, 15 to 30 nucleobases, 15 to 25 nucleobases, 15 to 20 nucleobases, 15 to 19 nucleobases, 15 to 18 nucleobases, 15 to 16 nucleobases, 16 to 20 nucleobases, 16 to 19 nucleobases, 16 to 18 nucleobases, 17 to 20 nucleobases, 17 to 19 nucleobases, or 18 to 20 nucleobases.

135. The pharmaceutical composition for use according to claim 127, wherein the pharmaceutical composition comprises the antisense oligomer, and wherein the antisense oligomer is a modified oligonucleotide comprising a sequence set forth in any one of SEQ ID NOs: 154-189 or 192-247.

136. The pharmaceutical composition for use according to claim 127, wherein the level of the processed mRNA encoding the UBE3A protein in the mammalian cell contacted with the antisense oligomer or the vector encoding the antisense oligomer is decreased by about 10% to about 99%, about 10% to about 95%, about 10% to about 90%, about 10% to about 80%, about 10% to about 70%, about 10% to about 60%, about 10% to about 50%, about 10% to about 40%, about 10% to about 30%, about 10% to about 20%, about 20% to about 90%, about 20% to about 80%, about 20% to about 70%, about 20% to about 60%, about 20% to about 50%, about 20% to about 40%, about 20% to about 30%, about 30% to about 80%, about 30% to about 70%, about 30% to about 60%, about 30% to about 50%, about 40% to about 70%, about 40% to about 60%, about 40% to about 50%, about 50% to about 99%, about 50% to about 95%, about 50% to about 90%, about 50% to about 80%, about 50% to about 70%, about 50% to about 60%, about 60% to about 99%, about 60% to about 95%, about 60% to about 90%, about 60% to about 80%, about 60% to about 70%, about 70% to about 99%, about 70% to about 95%, about 70% to about 90%, about 70% to about 80%, about 80% to about 99%, about 80% to about 95%, about 80% to about 90%, about 90% to about 99%, about 90% to about 95%, or about 95% to about 99%, as compared to an otherwise same mammalian cell not contacted with the antisense oligomer or the vector encoding the antisense oligomer.

137. The pharmaceutical composition for use according to claim 127, wherein the method reduces a level of the UBE3A protein in the mammalian cell, and
wherein the level of the UBE3A protein in the mammalian cell contacted with the antisense oligomer or the vector encoding the antisense oligomer is decreased by about 10% to about 99%, about 10% to about 95%, about 10% to about 90%, about 10% to about 80%, about 10% to about 70%, about 10% to about 60%, about 10% to about 50%, about 10% to about 40%, about 10% to about 30%, about 10% to about 20%, about 20% to about 90%, about 20% to about 80%, about 20% to about 70%, about 20% to about 60%, about 20% to about 50%, about 20% to about 40%, about 20% to about 30%, about 30% to about 80%, about 30% to about 70%, about 30% to about 60%, about 30% to about 50%, about 40% to about 70%, about 40% to about 60%, about 40% to about 50%, about 50% to about 99%, about 50% to about 95%, about 50% to about 90%, about 50% to about 80%, about 50% to about 70%, about 50% to about 60%, about 60% to about 99%, about 60% to about 95%, about 60% to about 90%, about 60% to about 80%, about 60% to about 70%, about 70% to about 99%, about 70% to about 95%, about 70% to about 90%, about 70% to about 80%, about 80% to about 99%, about 80% to about 95%, about 80% to about 90%, about 90% to about 99%, about 90% to about 95%, or about 95% to about 99%, as compared to an otherwise same mammalian cell not contacted with the antisense oligomer or the vector encoding the antisense oligomer.

138. The pharmaceutical composition for use according to claim 127, wherein the antisense oligomer reduces a level of the processed mRNA encoding the UBE3A protein in the mammalian cell contacted with the antisense oligomer or the vector encoding the antisense oligomer by at most about 75%, at most about 70%, at most about 60%, at most about 50%, at most about 40%, at most about 30%, or at most about 20%, as compared to an otherwise same mammalian cell not contacted with the antisense oligomer or the vector encoding the antisense oligomer.

139. The pharmaceutical composition for use according to claim 127, wherein the antisense oligomer reduces a level of the UBE3A protein in the mammalian cell contacted with the antisense oligomer or the vector encoding the antisense oligomer by at most about 75%, at most about 70%, at most about 60%, at most about 50%, at most about 40%, at most about 30%, or at most about 20%, as compared to an otherwise same mammalian cell not contacted with the antisense oligomer or the vector encoding the antisense oligomer.

140. The pharmaceutical composition for use according to claim 127, wherein the mammalian cell is ex vivo.

141. The pharmaceutical composition for use according to claim 127, wherein the mammalian cell has a duplication of a genomic region that encompasses the UBE3A gene encoding the UBE3A protein.

142. The pharmaceutical composition for use according to claim 127, wherein the mammalian cell is a human cell, and wherein the mammalian cell has a duplication of chromosome 15q11.2-q13.1, and/or
wherein the mammalian cell is obtained from a human subject suffering from Dup15q syndrome, or a progeny of a sample cell obtained from the human subject.

143. An antisense oligomer that comprises a sequence with at least 80% identity to the sequence set forth in any one of SEQ ID NOs: 1-92.

144. A pharmaceutical composition, comprising:
(a) a pharmaceutically acceptable excipient or carrier; and
(b) a therapeutic agent comprising the antisense oligomer of claim 143 or a vector encoding the antisense oligomer of claim 143, wherein the therapeutic agent is configured to reduce a level of a processed mRNA transcript encoding a UBE3A protein in a mammalian cell upon contact with the mammalian cell.

145. A method of treating a disease or condition in a subject in need thereof by reducing expression of a UBE3A protein in cells of the subject, comprising contacting to the cells of the subject the pharmaceutical composition comprising an antisense oligomer that comprises a sequence with at least 80% identity to the sequence set forth in any one of SEQ ID NOs: 1-92.
